(19) United States
(12) Patent Application Publication
(10) Pub. No.: US 2026/0184775 A1
(43) Pub. Date: Jul. 2, 2026

(54) CROSS-SPECIES ANTI-LATENT TGF-BETA 1 ANTIBODIES AND METHODS OF USE

(71) Applicant: Chugai Seiyaku Kabushiki Kaisha, Tokyo (JP)

(72) Inventor: Masakazu KANAMORI, Singapore (SG)

(73) Assignee: Chugai Seiyaku Kabushiki Kaisha, Tokyo (JP)

(21) Appl. No.: 19/565,798

(22) Filed: Mar. 13, 2026

Related U.S. Application Data
(63) Continuation of application No. 18/541,602, filed on Dec. 15, 2023, now abandoned.
Application No. 18/541,602 is a division of application No. 16/971,924, now abandoned.

(30) Foreign Application Priority Data
Feb. 23, 2018 (JP) 2018-030344

(51) Int. Cl. C07K 16/22; A61K 39/00

(52) U.S. Cl. CPC C07K 16/22; A61K 2039/505; C07K 2317/33; C07K 2317/76; C07K 2317/92

(57) ABSTRACT

The present invention provides cross-species anti-latent TGF-beta 1 antibodies. The present inventors have conducted diligent studies and consequently created cross-species anti-latent TGF-beta 1 antibodies which inhibit a protease mediated activation of TGF-beta 1 but do not inhibit integrin mediated activation of latent TGF-beta 1. It has been reported that latent TGF-beta is cleaved to release active TGF-beta by proteases. However, surprisingly, the present inventors have found that the anti-TGF-beta 1 antibodies inhibit a protease mediated activation of latent TGF-beta 1 without inhibiting protease mediated cleavage of the LAP region of latent TGF-beta 1, and have in vivo anti-fibrotic effects as well. Further, antibodies of the invention are useful for the diagnosis or treatment of cancer, and can also be used in combination with immune check point inhibitors.

### CROSS-REFERENCE TO RELATED APPLICATIONS

This application is a continuation of U.S. patent application Ser. No. 18/541,602, filed Dec. 15, 2023, which is a divisional of U.S. patent application Ser. No. 16/971,924, filed Aug. 21, 2020, now abandoned, which is a U.S. National Phase of PCT Application No. PCT/JP2019/006688, filed Feb. 22, 2019, which claims the benefit of Japanese Patent Application No. 2018-030344, filed Feb. 23, 2018, each of which is incorporated herein by reference in its entirety.

### REFERENCE TO SEQUENCE LISTING SUBMITTED ELECTRONICALLY

The content of the electronically submitted sequence listing (Name: 6663_0393 Sequence_Listing.xml; Size: 116,707 bytes; and Date of Creation: Mar. 12, 2026) is incorporated herein by reference in its entirety.

### TECHNICAL FIELD

The present invention relates to anti-TGF-beta 1 antibodies and methods of using the same.

### BACKGROUND ART

Transforming growth factor-beta (transforming growth factor beta; TGF-beta) is a member of the TGF-beta superfamily of cytokines, which consists of TGF-betas, activins, inhibins, Nodal, bone morphogenetic proteins (BMPs), anti-Mullerian hormone (AMH), as well as growth and differentiation factors (GDFs). Members of this superfamily are dimeric proteins with conserved structures and have pleiotropic functions in vitro and in vivo (NPL 1, 2). The TGF-betas are involved in many cellular processes, including growth inhibition, cell migration, invasion, epithelial-mesenchymal transition (EMT), extracellular matrix (ECM) remodeling, and immune-suppression (NPL 3). However, although normally dynamically regulated and involved in maintenance of tissue homeostasis, TGF-betas are often chronically overexpressed in disease states, including cancer, fibrosis, and inflammation, and this excessive production of TGF-beta drives disease progression by modulating cell growth, migration, or phenotype.

Three separate TGF-beta isoforms (TGF-beta 1, TGF-beta 2, and TGF-beta 3) have been identified in mammals, and share 70-82% homology at the amino acid level (NPL 4). All three TGF-beta isoforms bind to TGF-beta receptor type 2 (TGFR2) as homodimers (their active form); TGFR2 then recruits and activates TGF-beta receptor type 1 (TGFR1) to activate receptor signaling (NPL 5). However, expression levels of the three isoforms vary depending on the tissue (NPL 6), and their functions are distinct, as demonstrated by the phenotypes of knockout mice (NPL 7-11).

Like other members of the TGF-beta superfamily, TGF-beta is synthesized as a precursor protein, which forms a homodimer that interacts with its latency-associated peptide (LAP) and a latent TGF-beta-binding protein (LTBP) to form a larger complex called the large latent complex (LLC). The TGF-beta gene encodes a preproprotein sequence consisting of a signal peptide, a propeptide that ends with a proprotein convertase (PPC) cleavage site, and the mature TGF-beta sequence. Furin hydrolyzes the PPC cleavage site, creating separate TGF-beta- and propeptide-derived homodimers. The two homodimers remain noncovalently associated and are secreted. This latent complex keeps TGF-beta in an inactive form that is incapable of binding to its receptors (NPL 12, 13). The TGF-beta activation process involves the release of the LLC from the ECM, followed by further proteolysis of LAP to release active TGF-beta to its receptors (NPL 3). Latent TGF-beta is cleaved to release active TGF-beta by a wide range of proteases, including plasmin (PLN), plasma kallikrein (PLK), matrix metalloproteinase (MMP) 2, and MMP9 (NPL 14), and by thrombospondin 1 (TSP-1) (NPL 15). Without wishing to be bound by any theory, MMP2, as well as MMP9, proteolytically cleaves latent TGF-beta 1 and release mature TGF-beta 1 from latent form. Both MMP2 and MMP9 is synthesized as inactive pro-MMP. Pro-MMP2 is activated by a complex of membrane type 1 MMP (MT1-MMP/MMP14) and tissue inhibitor of metalloproteinase 2 (TIMP-2). Pro-MMP9 is activated through an interacting protease cascade involving plasmin and stromelysin 1 (MMP-3). Plasmin generates active MMP-3 from its zymogen. Active MMP-3 cleaves the propeptide from the 92-kDa pro-MMP-9, yielding an 82-kDa enzymatically active enzyme. The cleavage site of MMPs are not specifically determined; however, it is reported that MMP3 specifically cleaves the site between 79 Ala and 80 Leu of latent TGF-beta, so as to activate TGF-beta (WO2005/023870). Alternatively, upon mechanical stretch, integrins can activate TGF-beta by binding to the RGD motif present in LAP to induce the release of mature TGF-beta from its latent complex (NPL 16, 17).

After activation, the dimeric TGF-beta ligand binds to the extracellular domains of type I and type II receptors and induces close proximity, placing the intracellular serine/threonine kinase domains of the receptors in a conformation that facilitates the phosphorylation and subsequent activation of the type I receptor. This activation of the type I receptor leads to the propagation of signaling by at least two seemingly independent routes: the SMAD-dependent canonical pathway and the SMAD-independent or non-canonical pathway. In the SMAD-dependent pathway, activation of TGFR1 (also known as ALK5) leads to phosphorylation of SMAD proteins. SMAD2 and SMAD3 are substrates of TGFR1. Upon phosphorylation by the receptor, SMADs together with the common mediator SMAD4 translocate to the nucleus, where they interact with other transcription factors to regulate transcriptional responses (NPL 18). In the non-canonical pathway, the activated TGF-beta receptor complex transmits a signal through other factors, such as tumor necrosis factor (TNF) receptor-associated factor 4 (TRAF4), TRAF6, TGF-beta-activated kinase 1 (TAK1, also known as MAP3K7), p38 mitogen-activated protein kinase (p38 MAPK), RHO, phosphoinositide 3-kinase (PI3K), AKT (also known as protein kinase B), extracellular signal-regulated kinase (ERK), JUN N-terminal kinase (INK), or nuclear factor-kappa B (NF-kappa B). Thus, cellular responses to TGF-beta signaling result from the dynamic combination of canonical and non-canonical signaling cascades.

Fibrosis, or the accumulation of ECM molecules that make up scar tissue, is a common feature of chronic tissue injury. Pulmonary fibrosis, renal fibrosis, and hepatic cirrhosis are among the more common fibrotic diseases, which in aggregate represent a huge unmet clinical need. TGF-beta strongly promotes generation of the extracellular matrices of mesenchymal cells, while at the same time it suppresses the growth of epithelial cells, which contributes to the pathogenesis of sclerotic diseases. Overexpression of the active form of TGF-beta 1 in the liver of transgenic mice is sufficient to induce fibrotic disease in multiple organs (NPL 19). On the other hand, TGF-beta also plays an important role in maintaining our health. For example, TGF-beta suppresses excessive generation of proteases in the lung and prevents the destruction of lung tissue that leads to emphysema. Also, mice with deleted TGF-beta 1 show prenatal lethality (around 50% at 10.5 days post coitus) or their offspring die shortly after birth, with massive inflammatory lesions seen in many organs, including the lungs (vasculitis, perivascular cuffing, and interstitial pneumonia) and heart (endocarditis and myocarditis), which suggests that TGF-beta 1 plays a crucial role in maintaining immune homeostasis (NPL 7).

Results of studies using a neutralizing antibody to TGF-beta and animal models revealed that sclerotic diseases can be prevented or cured by suppressing the action of TGF-beta. As TGF-beta is produced as a precursor protein, there are several reported approaches to prevent activation from the latent form. Another method of preventing activation from the latent form is to use an inhibitor or antibody that binds to latent TGF-beta to block cleavage by proteases, such as PLK and PLN. Several antibodies that use this method of suppressing TGF-beta activation were reported as preventing or treating hepatic fibrosis/cirrhosis (PTL 1). In addition, there have been some documents mentioning anti-LAP antibodies for treating cancer (PTL 2), and TGF beta 1-binding immunoglobulins for treating TGF beta 1-related disorders (PTL 3).

### CITATION LIST

### Patent Literature

[PTL 1] WO 2011102483
[PTL 2] WO 2016115345
[PTL 3] WO 2017156500

### Non Patent Literature

[NPL 1] McCartney-Francis, N. L. et al. Int. Rev. Immunol. 16, 553-580 (1998)
[NPL 2] Massague, J. Annu. Rev. Biochem. 67, 753-791 (1998)
[NPL 3] Derynck, R. & Miyazono, K. Cold Spring Harbor Press (2008)
[NPL 4] Yu, L. et al. Kidney Int. 64, 844-856 (2003).
[NPL 5] Xu, P., Liu, J. & Derynck, R. et al. FEBS Lett. 586, 1871-1884 (2012).
[NPL 6] Millan, F. A. et al. Development 111, 131-143 (1991).
[NPL 7] Kulkarni, A. B. et al. Proc. Natl Acad. Sci. USA 90, 770-774 (1993).
[NPL 8] Shull, M. M. et al. Nature 359, 693-699 (1992).
[NPL 9] Dickson, M. C. et al. Development 121, 1845-1854 (1995).
[NPL 10] Sanford, L. P. et al. Development 124, 2659-2670 (1997).
[NPL 11] Proetzel, G. et al. Nature Genet. 11, 409-414 (1995).
[NPL 12] Dubois, C. M. et al. J. Biol. Chem. 270, 10618-10624 (1995)
[NPL 13] Nunes, I. et al. J. Am. Optom. Assoc. 69, 643-648 (1998)
[NPL 14] Annes, J. et al. J. Cell Sci. 116, 217-224 (2003).
[NPL 15] Schultz-Cherry, S. et al. J. Biol. Chem. 269, 26775-26782 (1994).
[NPL 16] Munger, J. S. et al. Cell 96, 319-328 (1999).
[NPL 17] Shi, M. et al. Nature 474, 343-349 (2011).
[NPL 18] Shi, Y. & Massague, et al. Cell 113, 685-700 (2003).
[NPL 19] Sanderson, N. et al. Proc. Natl Acad. Sci. USA 92, 2572-2576 (1995).

### SUMMARY OF INVENTION

### Technical Problem

An object of the invention is to provide cross-species anti-latent TGF-beta 1 antibodies which inhibit a protease mediated activation of latent TGF-beta 1 and have in vivo anti-fibrotic effects. The invention also provides anti-latent TGF-beta antibodies which inhibit a protease mediated activation of latent TGF-beta 1 without inhibiting a protease mediated cleavage of the LAP region of latent TGF-beta 1, and anti-latent TGF-beta antibodies which do not inhibit integrin mediated activation of latent TGF-beta 1.

### Solution to Problem

The present inventors have conducted diligent studies under the situations as described above and consequently created anti-TGF-beta 1 antibodies which inhibit a protease mediated activation of TGF-beta 1. It has been reported that latent TGF-beta is cleaved to release active TGF-beta by proteases. However, surprisingly, the present inventors have found that the anti-TGF-beta 1 antibodies inhibit a protease mediated activation of latent TGF-beta 1 without inhibiting protease mediated cleavage of the LAP region of latent TGF-beta 1, and have in vivo anti-fibrotic effects as well.

The present invention provides:
[1] An anti-latent TGF-beta 1 antibody, which comprises:
   (1) HVR-H1, HVR-H2 and HVR-H3 comprising the amino acid sequences of SEQ ID NOs: 5, 6, 7, respectively, and HVR-L1, HVR-L2 and HVR-L3 comprising the amino acid sequences of SEQ ID NOs: 8, 9, 10, respectively;
   (2) HVR-H1, HVR-H2 and HVR-H3 comprising the amino acid sequences of SEQ ID NOs: 11, 12, 13, respectively, and HVR-L1, HVR-L2 and HVR-L3 comprising the amino acid sequences of SEQ ID NOs: 14, 15, 16, respectively; or
   (3) HVR-H1, HVR-H2 and HVR-H3 comprising the amino acid sequences of SEQ ID NOs: 17, 18, 19, respectively, and HVR-L1, HVR-L2 and HVR-L3 comprising the amino acid sequences of SEQ ID NOs: 20, 21, 22, respectively.
[2] An anti-latent TGF-beta 1 antibody that binds to the same epitope as a reference antibody, wherein the reference antibody comprises:
   (1) HVR-H1, HVR-H2 and HVR-H3 comprising the amino acid sequences of SEQ ID NOs: 5, 6, 7, respectively, and HVR-L1, HVR-L2 and HVR-L3 comprising the amino acid sequences of SEQ ID NOs: 8, 9, 10, respectively;
   (2) HVR-H1, HVR-H2 and HVR-H3 comprising the amino acid sequences of SEQ ID NOs: 11, 12, 13, respectively, and HVR-L1, HVR-L2 and HVR-L3 comprising the amino acid sequences of SEQ ID NOs: 14, 15, 16, respectively; or
   (3) HVR-H1, HVR-H2 and HVR-H3 comprising the amino acid sequences of SEQ ID NOs: 17, 18, 19, respectively, and HVR-L1, HVR-L2 and HVR-L3 comprising the amino acid sequences of SEQ ID NOs: 20, 21, 22, respectively.
[3] The anti-latent TGF-beta 1 antibody according to [2], wherein the antibody binds to human latent TGF-beta 1 and mouse latent TGF-beta 1.
[4] The anti-latent TGF-beta 1 antibody according to [2] or [3], wherein the antibody binds to latent TGF-beta 1 forming cell surface latent TGF beta 1, large latent complex (LLC), and/or small latent complex (SLC).
[5] The anti-latent TGF-beta 1 antibody according to any one of [2] to [4], wherein the antibody binds to the latency-associated peptide (LAP) region of latent TGF-beta 1.
   [5-2] The anti-latent TGF-beta 1 antibody according to any one of [2] to [4], wherein the antibody binds to the latency-associated peptide (LAP).
[6] The anti-latent TGF-beta 1 antibody according to any one of [2] to [5-2], where in the antibody does not bind to mature TGF-beta 1.
[7] The anti-latent TGF-beta 1 antibody according to any one of [2] to [6], wherein the antibody inhibits a protease mediated activation of latent TGF-beta 1 without inhibiting a protease mediated cleavage of the LAP region of latent TGF-beta 1.
   [7-2] The anti-latent TGF-beta 1 antibody according to any one of [2] to [6], wherein the antibody inhibits a protease mediated release of mature TGF-beta 1 from latent TGF-beta 1 without inhibiting a protease mediated cleavage of the LAP region of latent TGF-beta 1.
[8] The anti-latent TGF-beta 1 antibody according to [7] or [7-2], wherein the protease is selected from the group consisting of plasmin (PLN), plasma kallikrein (PLK), matrix metalloproteinase (EMP) 2 and MMP9.
[9] The anti-latent TGF-beta 1 antibody according to any one of [1] to [8], wherein the antibody does not inhibit integrin mediated activation of latent TGF-beta 1.
[10] The anti-latent TGF-beta 1 antibody according to any one of [1] to [9], wherein the antibody is a human, humanized or chimeric antibody.
[11] The anti-latent TGF-beta 1 antibody according to any one of [1] to [9], wherein the antibody comprises an Fc region with reduced binding activity towards an Fc gamma receptor.
   [11-2] The antibody according to any one of [1] to [11], wherein the antibody is monoclonal antibody.
   [11-3] The antibody according to any one of [1] to [11-2], wherein the antibody is an anti-latent TGF-beta 1 antibody fragment.
[12] An isolated nucleic acid encoding the antibody of any one of [1] to [11-3].
[13]A host cell comprising the nucleic acid of [12].
[14]A method of producing an antibody comprising culturing the host cell of [13] so that the antibody is produced.
   [14-2] The method according to [14], further comprising recovering the antibody from the host cell.
[15]A pharmaceutical composition comprising the anti-latent TGF-beta 1 antibody according to any one of [1] to [11-3] and a pharmaceutically acceptable carrier.
[16] The antibody according to any one of [1] to [11-3] for use as a medicament.
[17] The antibody according to any one of [1] to [11-3] for use in treating fibrosis.
[18] The antibody according to any one of [1] to [11-3] for use in inhibiting a protease mediated activation of latent TGF-beta 1 without inhibiting a protease mediated cleavage of the LAP region of latent TGF-beta 1.
[19] The antibody according to any one of [1] to [11-3] for use in inhibiting a protease mediated release of mature TGF-beta 1 from latent TGF-beta 1 without inhibiting a protease mediated cleavage of the LAP region of latent TGF-beta 1.
[20] Use of the antibody according to any one of [1] to [11-3] in the manufacture of a medicament for treatment of fibrosis.
[21]A method of treating a subject having a fibrosis comprising administering to the subject an effective amount of the anti-latent TGF-beta 1 antibody according to any one of [1] to [11-3].
[22] The method according to [21], wherein the fibrosis is renal fibrosis or pulmonary fibrosis.
[23]A method of inhibiting a protease mediated activation of latent TGF-beta 1 without inhibiting a protease mediated cleavage of the LAP region of latent TGF-beta 1 in an individual comprising administering to the individual an effective amount of an antibody of any one of [1] to [11-3] to inhibit a protease mediated activation of latent TGF-beta 1 without inhibiting a protease mediated cleavage of the LAP region of latent TGF-beta 1.
[24]A method of inhibiting a protease mediated release of mature TGF-beta 1 from latent TGF-beta 1 without inhibiting a protease mediated cleavage of the LAP region of latent TGF-beta 1 in an individual comprising administering to the individual an effective amount of an antibody of any one of [1] to [11-3] to inhibit a protease mediated release of mature TGF-beta 1 from latent TGF-beta 1 without inhibiting a protease mediated cleavage of the LAP region of latent TGF-beta 1.
[25] An anti-latent TGF-beta 1 antibody which stabilizes the structure of the LAP region of latent TGF-beta 1 without inhibiting a protease mediated cleavage of the LAP region of latent TGF-beta 1, wherein the antibody comprises:
   (1) HVR-H1, HVR-H2 and HVR-H3 comprising the amino acid sequences of SEQ ID NOs: 5, 6, 7, respectively, and HVR-L1, HVR-L2 and HVR-L3 comprising the amino acid sequences of SEQ ID NOs: 8, 9, 10, respectively;
   (2) HVR-H1, HVR-H2 and HVR-H3 comprising the amino acid sequences of SEQ ID NOs: 11, 12, 13, respectively, and HVR-L1, HVR-L2 and HVR-L3 comprising the amino acid sequences of SEQ ID NOs: 14, 15, 16, respectively; or
   (3) HVR-H1, HVR-H2 and HVR-H3 comprising the amino acid sequences of SEQ ID NOs: 17, 18, 19, respectively, and HVR-L1, HVR-L2 and HVR-L3 comprising the amino acid sequences of SEQ ID NOs: 20, 21, 22, respectively.
[26] An anti-latent TGF-beta 1 antibody that binds to the LAP region of latent TGF-beta 1, wherein the antibody stabilizes the structure of the LAP region of latent TGF-beta 1 which has been cleaved by a protease, wherein the antibody comprises:
   (1) HVR-H1, HVR-H2 and HVR-H3 comprising the amino acid sequences of SEQ ID NOs: 5, 6, 7, respectively, and HVR-L1, HVR-L2 and HVR-L3 comprising the amino acid sequences of SEQ ID NOs: 8, 9, 10, respectively;
   (2) HVR-H1, HVR-H2 and HVR-H3 comprising the amino acid sequences of SEQ ID NOs: 11, 12, 13, respectively, and HVR-L1, HVR-L2 and HVR-L3 comprising the amino acid sequences of SEQ ID NOs: 14, 15, 16, respectively; or
   (3) HVR-H1, HVR-H2 and HVR-H3 comprising the amino acid sequences of SEQ ID NOs: 17, 18, 19, respectively, and HVR-L1, HVR-L2 and HVR-L3 comprising the amino acid sequences of SEQ ID NOs: 20, 21, 22, respectively.
[27] An anti-latent TGF-beta 1 antibody that binds to the LAP region of latent TGF-beta 1, wherein the antibody (a) inhibits a protease mediated release of mature TGF-beta 1 from latent TGF-beta 1; and (b) allows a protease to cleave the LAP region while the anti-latent TGF-beta 1 antibody binds to the LAP region of latent TGF-beta 1, and wherein the antibody comprises:
   (1) HVR-H1, HVR-H2 and HVR-H3 comprising the amino acid sequences of SEQ ID NOs: 5, 6, 7, respectively, and HVR-L1, HVR-L2 and HVR-L3 comprising the amino acid sequences of SEQ ID NOs: 8, 9, 10, respectively;
   (2) HVR-H1, HVR-H2 and HVR-H3 comprising the amino acid sequences of SEQ ID NOs: 11, 12, 13, respectively, and HVR-L1, HVR-L2 and HVR-L3 comprising the amino acid sequences of SEQ ID NOs: 14, 15, 16, respectively; or
   (3) HVR-H1, HVR-H2 and HVR-H3 comprising the amino acid sequences of SEQ ID NOs: 17, 18, 19, respectively, and HVR-L1, HVR-L2 and HVR-L3 comprising the amino acid sequences of SEQ ID NOs: 20, 21, 22, respectively.

### BRIEF DESCRIPTION OF DRAWINGS

FIG. 1 shows the results of antibody binding to cell surface mouse latent TGF-beta 1 by FACS using Ba/F3 cells (FIG. 1A) or human TGF-beta 1 transfected FreeStyle™ 293-F cells (FIG. 1B). Anti-latent TGF-beta 1 antibodies bound to mouse cell surface latent TGF-beta 1 expressed on Ba/F3 cells and human cell surface latent TGF-beta 1 expressed on FreeStyle™ 293-F cells. IC17 represents an anti-KLH antibody as a negative control.

FIG. 2 shows the results of antibody activity against spontaneous mouse latent TGF-beta 1 activation. Spontaneous mouse latent TGF-beta 1 activation was suppressed by anti-latent TGF-beta 1 antibodies. IC17 represents an anti-KLH antibody as a negative control.

FIG. 3 shows the results of antibody activity against plasmin (PLN)-mediated mouse latent TGF-beta 1 activation. PLN-mediated mouse latent TGF-beta 1 activation was suppressed by anti-latent TGF-beta 1 antibodies. IC17 represents an anti-KLH antibody as a negative control.

FIG. 4 shows the results of antibody activity against plasmin (PLN)-mediated human latent TGF-beta 1 activation. PLN-mediated human latent TGF-beta 1 activation was suppressed by anti-latent TGF-beta 1 antibodies. IC17 represents an anti-KLH antibody as a negative control. Cam represents camostat which is a protease inhibitor used as a control.

FIG. 5 shows the results of antibody activity against matrix metalloproteinase (MMP) 2-mediated mouse latent TGF-beta 1 activation (FIG. 5A) and MMP9-mediated mouse latent TGF-beta 1 activation (FIG. 5B). MMP2 and 9-mediated mouse latent TGF-beta 1 activation was suppressed by anti-latent TGF-beta 1 antibodies. IC17 represents an anti-KLH antibody as a negative control. GM6001 is a MMP inhibitor used as a positive control.

FIG. 6 shows the result of antibody activity against PLN-mediated mouse latent TGF-beta 1 cleavage. Mouse latent TGF-beta 1 cleavage by PLN was not inhibited by TBA0946, TBA0947, and TBA1172. Cam represents camostat which is a protease inhibitor used as a control.

FIG. 7 shows the result of antibody activity against integrin-mediated mouse TGF-beta 1 activation in mouse PBMC. TBA0946 did not inhibit integrin-mediated latent TGF-beta 1 activation in mouse PBMC. TBA0947 and TBA1172 partially inhibited integrin-mediated TGF-beta 1 activation in mouse PBMC. RGE represents RGE peptide. RGD represents RGD peptide as a positive control.

FIG. 8 shows the result of antibody binding to mouse mature TGF-beta 1. TBA0946, TBA0947, and TBA1172 did not bind to mouse mature TGF-beta 1. The anti-mature TGF-beta antibody GC1008 was used as a positive control.

FIG. 9 shows the results of collagen type 1 alpha 1 (Col1a1) mRNA in kidney. Monoclonal antibodies were evaluated in a Unilateral Ureteral Obstruction (UUO) induced mouse renal fibrosis model. Sham operated group represents as a non-disease induced control. Col1a1 mRNA was suppressed by treatment with anti-latent TGF-beta 1 antibodies. IC17 is an anti-KLH antibody used as a negative control. GC1008 is an anti-mature TGF-beta antibody used as a positive control.

FIG. 10 shows the results of plasminogen activator inhibitor 1 (PAI-1) mRNA in kidney. Monoclonal antibodies were evaluated in a Unilateral Ureteral Obstruction (UUO) induced mouse renal fibrosis model. Sham operated group represents as a non-disease induced control. PAI-1 mRNA was suppressed by treatment with anti-latent TGF-beta 1 antibodies. IC17 is an anti-KLH antibody used as a negative control. GC1008 is an anti-mature TGF-beta antibody used as a positive control.

FIG. 11 shows the results of hydroxyproline content in kidney. Monoclonal antibodies were evaluated in a Unilateral Ureteral Obstruction (UUO) induced mouse renal fibrosis model. Sham operated group represents as a non-disease induced control. Kidney fibrosis was reduced by treatment with anti-latent TGF-beta 1 antibodies. IC17 is an anti-KLH antibody used as a negative control. GC1008 is an anti-mature TGF-beta antibody used as a positive control.

FIG. 12 shows the results of collagen type 1 alpha 1 (Col1a1) mRNA in lung. Monoclonal antibodies were evaluated in a Bleomycin (BLM) induced mouse pulmonary fibrosis model. Normal control (NC) represents as a non-disease induced control. Col1a1 mRNA was suppressed by TBA1172. IC17 is an anti-KLH antibody used as a negative control. GC1008 is an anti-mature TGF-beta antibody used as a control.

FIG. 13 shows the results of plasminogen activator inhibitor 1 (PAI-1) mRNA in lung. Monoclonal antibodies were evaluated in a Bleomycin (BLM) induced mouse pulmonary fibrosis model. Normal control (NC) represents as a non-disease induced control. PAI-1 mRNA was suppressed by TBA1172.IC17 is an anti-KLH antibody used as a negative control.

FIG. 14 shows the results of chemokine ligand 2 (CCL2) mRNA in lung. Monoclonal antibodies were evaluated in a Bleomycin (BLM) induced mouse pulmonary fibrosis model. Normal control (NC) represents as a non-disease induced control. GC1008 treatment enhanced inflammatory response but TBA1172 did not. IC17 is an anti-KLH antibody used as a negative control. GC1008 is an anti-mature TGF-beta antibody used as a control.

FIG. 15 shows the results of hydroxyproline content in lung. Monoclonal antibodies were evaluated in a Bleomycin (BLM) induced mouse pulmonary fibrosis model. Normal control (NC) represents as a non-disease induced control. Lung fibrosis was reduced by TBA1172. IC17 is an anti-KLH antibody used as a negative control.

FIG. 16 shows the results of antibody activity against (A) PLK and (B) PLN mediated latent TGF-beta 1 activation.

FIG. 17 shows the results of antibody activity against spontaneous activation of latent TGF-beta 1.

FIG. 18 shows the results of antibody activity against plasmin mediated latent TGF-beta 1 cleavage. Latent TGF-beta 1 cleavage by plasmin was only inhibited by TBA865 and TBA873 but not by TBS139 and TBS182. Cam represents camostat which is a protease inhibitor and IC17 represents an anti-KLH antibody (IC17) as a negative control antibody.

FIG. 19 shows the results of antibody activity against latent TGF-beta 1 activation in mouse PBMC. IC17 represents an anti-KLH antibody (IC17) as a negative control antibody and GC represents an anti-mature TGF-beta 1 antibody GC1008.

FIG. 20 shows Biacore results by tandem blocking assay. A saturating binding concentration of (A) TBS139 or (B) TBS182 was injected at time zero, followed by an injection of a competing antibody (TBS139, TBS182, TBA865, or TBA873) at 300s over latent-TGF beta 1 sensor surface.

FIG. 21 shows the expression levels of collagen type 1 alpha 1 mRNA in liver. Monoclonal antibodies were evaluated in a mouse model with NASH/liver fibrosis induced by a choline-deficient, L-amino acid-defined, high-fat diet (CDAHFD). CE-2 is a commercial standard diet. IC17 represents an anti-KLH (IC17) antibody as a negative control. GC1008 represents an anti-mature TGF-beta antibody (GC1008) as a positive control.

FIG. 22 shows the expression levels of collagen type 1 alpha 1 mRNA in kidney. Monoclonal antibodies were evaluated in a Unilateral Ureteral Obstruction (UUO)-induced renal fibrosis mouse model. A sham-operated group was used as a non-diseased control. IC17 represents an anti-KLH (IC17) antibody as a negative control. GC1008 represents an anti-mature TGF-beta antibody (GC1008) as a positive control.

FIG. 23 shows the hydroxyproline content in kidney after treatment with anti-latent TGF-beta 1 monoclonal antibodies (A) TBS139 and (B) TBS182. Antibodies were evaluated in a Unilateral Ureteral Obstruction (UUO)-induced renal fibrosis mouse model. The sham-operated group was used as a non-diseased control. IC17 represents an anti-KLH (IC17) antibody as a negative control. GC1008 represents an anti-mature TGF-beta antibody (GC1008) as a positive control.

FIG. 24 shows the expression levels of serpine 1 mRNA in lung after treatment with anti-latent TGF-beta 1 monoclonal antibodies (A) TBS139 and (B) TBS182. Antibodies were evaluated in a BLM-induced lung fibrosis mouse model. BLM and saline were instilled intratracheally. The saline-administered group was used as a non-diseased control. IC17 represents an anti-KLH (IC17) antibody as a negative control. GC1008 represents an anti-mature TGF-beta antibody (GC1008) as a positive control.

FIG. 25 shows the expression levels of CCL2 (MCP-1) mRNA in lung after treatment with anti-latent TGF-beta 1 monoclonal antibodies (A) TBS139 and (B) TBS182. Monoclonal antibodies were evaluated in a BLM-induced lung fibrosis mouse model. BLM and saline were instilled intratracheally. The saline-administered group was used as a non-diseased control. IC17 represents an anti-KLH (IC17) antibody as a negative control. GC1008 represents an anti-mature TGF-beta antibody (GC1008) as a positive control.

FIG. 26 shows the results of antibody activity against (A) MMP2 and (B) MMP9 protease mediated mouse latent TGF-beta 1 activation. Mouse MMP2 and MMP9 mediated mouse TGF-beta 1 activation was suppressed by anti-latent TGF-beta 1 antibodies.

FIG. 27 shows the results of antibody activity against (A) Prekallikrein (PLK) and (B) plasmin (PLN) protease mediated human latent TGF-beta 1 activation. PLK and PLN mediated human latent TGF-beta 1 activation was suppressed by anti-latent TGF-beta 1 antibodies. SLC represents latent TGF-beta1.

FIG. 28 shows the results of antibody activity against plasmin mediated human latent TGF-beta 1 cleavage. Human latent TGF-beta 1 cleavage by plasmin was only inhibited by TBA873 but not by TBA1300, TBA1314, and TBA1277. Cam represents camostat which is a protease inhibitor. IC17 represents anti-KLH Ab which was used as a negative control.

FIG. 29 shows the results of antibody binding to cell surface latent TGF-beta 1 by FACS using Ba/F3 cells or Free Style 293-F cells. A) TBS139 and TBS182 bind to cell surface mouse latent TGF-beta 1. However, TBA865 and TBA873 did not bind to it. B) TBA1277, TBA1300, and TBA1314 bind to cell surface human latent TGF-beta 1. However, TBA865 and TBA873 did not bind to it. IC17 represents anti-KLH antibody used as a negative control.

FIG. 30; (A) shows the results of antibody activity against MMP2 mediated mouse latent TGF-beta 1 cleavage. Mouse latent TGF-beta 1 cleavage by MMP2 was only inhibited by TBS139 and TBS182 but not by TBA865 and TBA873. GM represents GM6001 which is a MMP inhibitor. IC17 represents anti-KLH Ab which was used as a negative control. (B) shows the results of antibody activity against MMP9 mediated mouse latent TGF-beta 1 cleavage. Mouse latent TGF-beta 1 cleavage by MMP9 was only inhibited by TBS139 and TBS182 but not by TBA865 and TBA873. GM represents GM6001 which is a MMP inhibitor. IC17 represents anti-KLH Ab which was used as a negative control.

FIG. 31 shows the results of antibody activity against (A) MMP2 and (B) MMP9 protease mediated latent TGF-beta 1 activation. Mouse MMP2 and MMP9 mediated human latent TGF-beta 1 activation was suppressed by anti-latent TGF-beta 1 antibodies (TBA865, TBA873, TBA1300, and TBA1277). GM6001 is a MMP inhibitor. IC17 represents anti-KLH Ab which was used as a negative control. In this figure, “−” below the bars represents the absence of antibody addition.

### DESCRIPTION OF EMBODIMENTS

### I. Definitions

An “acceptor human framework” for the purposes herein is a framework comprising the amino acid sequence of a light chain variable domain (VL) framework or a heavy chain variable domain (VH) framework derived from a human immunoglobulin framework or a human consensus framework, as defined below. An acceptor human framework “derived from” a human immunoglobulin framework or a human consensus framework may comprise the same amino acid sequence thereof, or it may contain amino acid sequence changes. In some embodiments, the number of amino acid changes are 10 or less, 9 or less, 8 or less, 7 or less, 6 or less, 5 or less, 4 or less, 3 or less, or 2 or less. In some embodiments, the VL acceptor human framework is identical in sequence to the VL human immunoglobulin framework sequence or human consensus framework sequence.

“Affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Affinity can be measured by common methods known in the art, including those described herein. Specific illustrative and exemplary embodiments for measuring binding affinity are described in the following.

An “affinity matured” antibody refers to an antibody with one or more alterations in one or more hypervariable regions (HVRs), compared to a parent antibody which does not possess such alterations, such alterations resulting in an improvement in the affinity of the antibody for antigen.

The terms “anti-TGF-beta 1 antibody” and “an antibody that binds to TGF-beta 1” refer to an antibody that is capable of binding TGF-beta 1 with sufficient affinity such that the antibody is useful as a diagnostic and/or therapeutic agent in targeting TGF-beta 1. In one embodiment, “an antibody that binds to TGF-beta 1” is an antibody that specifically binds to TGF-beta 1. In one embodiment, the extent of binding of an anti-TGF-beta 1 antibody to an unrelated, non-TGF-beta 1 protein is less than about 10% of the binding of the antibody to TGF-beta 1 as measured, e.g., by a radioimmunoassay (RIA). In certain embodiments, an antibody that binds to TGF-beta 1 has a dissociation constant (Kd) of 1 micro M or less, 100 nM or less, 10 nM or less, 1 nM or less, 0.1 nM or less, 0.01 nM or less, or 0.001 nM or less (e.g. 10-8 M or less, e.g. from 10-8 M to 10-13 M, e.g., from 10-9 M to 10-13 M). In certain embodiments, an anti-TGF-beta 1 antibody binds to an epitope of TGF-beta 1 that is conserved among TGF-beta 1 from different species.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity. The term “antibody” also includes any antigen binding molecule which comprises variable heavy chain and/or variable light chain structure(s) of immunoglobulin.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)2; diabodies; linear antibodies; single-chain antibody molecules (e.g. scFv); and multispecific antibodies formed from antibody fragments.

An “antibody that binds to the same epitope” as a reference antibody refers to an antibody that blocks binding of the reference antibody to its antigen in a competition assay by 50% or more, and conversely, the reference antibody blocks binding of the antibody to its antigen in a competition assay by 50% or more. An exemplary competition assay is provided herein.

The term “chimeric” antibody refers to an antibody in which a portion of the heavy and/or light chain is derived from a particular source or species, while the remainder of the heavy and/or light chain is derived from a different source or species.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called alpha, delta, epsilon, gamma, and mu, respectively.

The term “cytotoxic agent” as used herein refers to a substance that inhibits or prevents a cellular function and/or causes cell death or destruction. Cytotoxic agents include, but are not limited to, radioactive isotopes (e.g., 211At, 131I, 125I, 90Y, 186Re, 188Re, 153Sm, 212Bi, 32P, 212Pb and radioactive isotopes of Lu); chemotherapeutic agents or drugs (e.g., methotrexate, adriamycin, vinca alkaloids (vincristine, vinblastine, etoposide), doxorubicin, melphalan, mitomycin C, chlorambucil, daunorubicin or other intercalating agents); growth inhibitory agents; enzymes and fragments thereof such as nucleolytic enzymes; antibiotics; toxins such as small molecule toxins or enzymatically active toxins of bacterial, fungal, plant or animal origin, including fragments and/or variants thereof, and the various antitumor or anticancer agents disclosed below.

“Effector functions” refer to those biological activities attributable to the Fc region of an antibody, which vary with the antibody isotype. Examples of antibody effector functions include: C1q binding and complement dependent cytotoxicity (CDC); Fc receptor binding; antibody-dependent cell-mediated cytotoxicity (ADCC); phagocytosis; down regulation of cell surface receptors (e.g. B cell receptor); and B cell activation.

An “effective amount” of an agent, e.g., a pharmaceutical formulation, refers to an amount effective, at dosages and for periods of time necessary, to achieve the desired therapeutic or prophylactic result.

The term “Fc region” herein is used to define a C-terminal region of an immunoglobulin heavy chain that contains at least a portion of the constant region. The term includes native sequence Fc regions and variant Fc regions. In one embodiment, a human IgG heavy chain Fc region extends from Cys226, or from Pro230, to the carboxyl-terminus of the heavy chain. However, the C-terminal lysine (Lys447) or glycine-lysine (residues 446-447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, MD, 1991.

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-H1(L1)-FR2-H2(L2)-FR3-H3(L3)-FR4.

The terms “full length antibody,” “intact antibody,” and “whole antibody” are used herein interchangeably to refer to an antibody having a structure substantially similar to a native antibody structure or having heavy chains that contain an Fc region as defined herein.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, Fifth Edition, NIH Publication 91-3242, Bethesda MD (1991), vols. 1-3. In one embodiment, for the VL, the subgroup is subgroup kappa I as in Kabat et al., supra. In one embodiment, for the VH, the subgroup is subgroup III as in Kabat et al., supra.

A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

The term “hypervariable region” or “HVR” as used herein refers to each of the regions of an antibody variable domain which are hypervariable in sequence (“complementarity determining regions” or “CDRs”) and/or form structurally defined loops (“hypervariable loops”) and/or contain the antigen-contacting residues (“antigen contacts”). Generally, antibodies comprise six HVRs: three in the VH (H1, H2, H3), and three in the VL (L1, L2, L3). Exemplary HVRs herein include:
(a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987));
(b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, MD (1991));
(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262: 732-745 (1996)); and
(d) combinations of (a), (b), and/or (c), including HVR amino acid residues 46-56 (L2), 47-56 (L2), 48-56 (L2), 49-56 (L2), 26-35 (H1), 26-35b (H1), 49-65 (H2), 93-102 (H3), and 94-102 (H3).

Unless otherwise indicated, HVR residues and other residues in the variable domain (e.g., FR residues) are numbered herein according to Kabat et al., supra.

An “immunoconjugate” is an antibody conjugated to one or more heterologous molecule(s), including but not limited to a cytotoxic agent.

An “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual or subject is a human.

An “isolated” antibody is one which has been separated from a component of its natural environment. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For review of methods for assessment of antibody purity, see, e.g., Flatman et al., J. Chromatogr. B 848:79-87 (2007).

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

“Isolated nucleic acid encoding an anti-TGF-beta 1 antibody” refers to one or more nucleic acid molecules encoding antibody heavy and light chains (or fragments thereof), including such nucleic acid molecule(s) in a single vector or separate vectors, and such nucleic acid molecule(s) present at one or more locations in a host cell.

The term “monoclonal antibody” as used herein refers to an antibody obtained from a population of substantially homogeneous antibodies, i.e., the individual antibodies composing the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

A “naked antibody” refers to an antibody that is not conjugated to a heterologous moiety (e.g., a cytotoxic moiety) or radiolabel. The naked antibody may be present in a pharmaceutical formulation.

“Native antibodies” refer to naturally occurring immunoglobulin molecules with varying structures. For example, native IgG antibodies are heterotetrameric glycoproteins of about 150,000 daltons, composed of two identical light chains and two identical heavy chains that are disulfide-bonded. From N- to C-terminus, each heavy chain has a variable region (VH), also called a variable heavy domain or a heavy chain variable domain, followed by three constant domains (CH1, CH2, and CH3). Similarly, from N- to C-terminus, each light chain has a variable region (VL), also called a variable light domain or a light chain variable domain, followed by a constant light (CL) domain. The light chain of an antibody may be assigned to one of two types, called kappa (kappa) and lambda (lambda), based on the amino acid sequence of its constant domain.

The term “package insert” is used to refer to instructions customarily included in commercial packages of therapeutic products, that contain information about the indications, usage, dosage, administration, combination therapy, contraindications and/or warnings concerning the use of such therapeutic products.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, Megalign (DNASTAR) software, or GENETYX (registered trademark) (Genetyx Co., Ltd.). Those skilled in the art can determine appropriate parameters for aligning sequences, including any algorithms needed to achieve maximal alignment over the full length of the sequences being compared.

The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, California, or may be compiled from the source code. The ALIGN-2 program should be compiled for use on a UNIX operating system, including digital UNIX V4.0D. All sequence comparison parameters are set by the ALIGN-2 program and do not vary. In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

The term “TGF-beta 1,” as used herein, refers to any native TGF-beta 1 from any vertebrate source, including mammals such as primates (e.g. humans) and rodents (e.g., mice and rats), unless otherwise indicated. The term encompasses “full-length” unprocessed TGF-beta 1 as well as any form of TGF-beta 1 that results from processing in the cell. The term also encompasses naturally occurring variants of TGF-beta 1, e.g., splice variants or allelic variants. The amino acid sequence of an exemplary human TGF-beta 1 preproprotein is shown in SEQ ID NO: 1 (NCBI RefSeq: NP_000651.3) and the nucleic acid sequence encoding an exemplary human TGF-beta 1 is shown in SEQ ID NO: 2 (NCBI RefSeq: NM_000660.6). The amino acid sequence of an exemplary mouse TGF-beta 1 preproprotein is shown in SEQ ID NO: 3 (NCBI RefSeq: NP_035707.1) and and the nucleic acid sequence encoding an exemplary mouse TGF-beta 1 is shown in SEQ ID NO: 4 (NCBI RefSeq: NM_011577.2). The term “TGF-beta 1” encompasses both latent TGF-beta 1 and mature TGF-beta 1.

The term “latent TGF-beta 1”, as used herein, refers to any TGF-beta 1 which forms a latent TGF-beta 1 complex (“cell surface latent TGF-beta 1”, LLC or SLC (see below)) and/or which is incapable of binding to its receptors. Transforming growth factor-beta 1 (TGF-beta 1) is a member of TGF-beta, which is a member of TGF-beta superfamily. Like other members of TGF-beta superfamily, TGF-beta is synthesized as a precursor protein, which forms a homodimer that interacts with its latency-associated peptide (LAP) and a latent TGF-beta-binding protein (LTBP), forming a larger complex called the large latent complex (LLC). The amino acid sequence of an exemplary latent human TGF-beta 1 (TGF-beta homodimer and its LAP) is amino acids 30-390 of SEQ ID NO: 1. The amino acid sequence of and exemplary mouse latent TGF-beta 1 (TGF-beta homodimer and its LAP) is amino acids 30-390 of SEQ ID NO: 3.

A complex formed from the TGF-beta homodimer and its LAP is called Small Latent Complex (SLC). This latent complex keeps TGF-beta in an inactive form, which is incapable of binding to its receptors. SLC may be covalently linked to an additional protein, latent TGF-beta binding protein (LTBP), forming the large latent complex (LLC). There are four different LTBP isoforms known, LTBP-1, LTBP-2, LTBP-3 and LTBP-4. It has been reported that LTBP-1, LTBP-3 and LTBP-4 bind to SLC (See, e.g., Rifkin et al., J Biol Chem. 2005 Mar. 4; 280(9):7409-12). SLC may also be covalently linked to other additional proteins, such as glycoprotein A repetitions predominant (GARP) or leucine-rich repeat-containing protein 33 (LRRC33). GARP and LRRC have a transmembrane domain and associate with LAP on the cell surface (See, e.g., Wang et al., Mol Biol Cell. 2012 March; 23(6):1129-39). As to LLCs, it is reported that LLCs associate covalently with the extracellular matrix (ECM) via the N-termini of the LTBPs (See, e.g., Saharinen et al., Cytokine Growth Factor Rev. 1999 June; 10(2):99-117.). In some embodiments, latent TGF-beta 1 associated with the ECM on a cell surface is referred to as “cell surface latent TGF-beta 1”.

The term “active TGF-beta 1”, “mature TGF-beta 1”, or “active mature TGF-beta 1”, as used herein, refers to any TGF-beta 1 homodimer which does not form a latent TGF-beta 1 complex (LLC or SLC) and which is capable of binding to its receptors. The TGF-beta 1 activation process involves the release of the LLC from the ECM, followed by further proteolysis of LAP to release active TGF-beta to its receptors. Wide range of proteases including plasmin (PLN), prekallikrein (PLK), matrix metalloproteinase (MMP) 2, MMP9, MMP13, MMP14, Thrombin, Tryptase and Calpain are known to cleave latent TGF-beta and release active TGF-beta. These proteases may be collectively called “(latent) TGF-beta-cleaving proteases” or “(latent) TGF-beta 1-cleaving proteases” in the context of the present invention. In addition to proteases, thrombospondin 1 (TSP-1), Neuropilin-1 (Nrp1), ADAMSTS1 and F-spondin activate latent TGF-beta. Alternatively, upon mechanical stretch, integrins can activate TGF-beta by binding to the RGD motif present in LAP and inducing the release of mature TGF-beta from its latent complex.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, antibodies of the invention are used to delay development of a disease or to slow the progression of a disease.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). (See, e.g., Kindt et al. Kuby Immunology, 6th ed., W.H. Freeman and Co., page 91 (2007).) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

### II. Compositions and Methods

In one aspect, the invention is based, in part, on anti-TGF-beta 1 antibodies and uses thereof. In certain embodiments, antibodies that bind to TGF-beta 1 are provided. Antibodies of the invention are useful, e.g., for the diagnosis or treatment of fibrosis, preferably myocardial fibrosis, pulmonary fibrosis, liver fibrosis, renal fibrosis, skin fibrosis, ocular fibrosis and myelofibrosis. Antibodies of the invention are also useful, e.g., for the diagnosis or treatment of cancer. In some embodiments, antibodies of the invention can be used in combination with immune check point inhibitors, e.g., an inhibitor of CTLA-4, PD-1, PD-L1, PD-L2, CD160, CD57, CD244, LAG-3, CD272, KLRG1, CD26, CD39, CD73, CD305, TIGIT, TIM-3, or VISTA. In some embodiments, immune check point inhibitors are, e.g., an anti-CTLA-4 antibody, an anti-PD-1 antibody, an anti-PD-L1 antibody, an anti-PD-L2 antibody, an anti-CD160 antibody, an anti-CD57 antibody, an anti-CD244 antibody, an anti-LAG-3 antibody, an anti-CD272 antibody, an anti-KLRG1 antibody, an anti-CD26 antibody, an anti-CD39 antibody, an anti-CD73 antibody, an anti-CD305 antibody, an anti-TIGIT antibody, an anti-TIM-3 antibody, or an anti-VISTA antibody. Preferably, the immune check point inhibitor is an anti-PD-1 antibody or an anti-PD-L1 antibody. In some embodiments, the anti-PD-1 antibody is Nivolumab, Pembrolizumab, or Cemiplimab. In some embodiments, the anti-PD-L1 antibody is Atezolizumab, Avelumab, or Durvalumab, preferably Atezolizumab. In some embodiments, a combination therapy comprising an anti-TGFbeta antibody of the invention and an immune check point inhibitor has additive or synergistic efficacy, e.g., additive or synergistic antitumor effect, compared to the anti-TGFbeta antibody monotherapy or the immune check point inhibitor monotherapy.

### A. Exemplary Anti-TGF-beta 1 Antibodies

In one aspect, the invention provides isolated antibodies that bind to TGF-beta 1. In certain embodiments, an anti-TGF-beta 1 antibody binds to latent TGF-beta 1. In further embodiments, the anti-TGF-beta 1 antibody binds to latent associated protein (LAP) region of the latent TGF-beta 1. An example of LAP region comprises amino acids 30-278 of a human TGF-beta 1 preproprotein (SEQ ID NO: 1). LAP is a component of latent TGF-beta 1, as described above. In some embodiments, an anti-TGF-beta 1 antibody binds to latent TGF-beta 1 with an affinity or binding activity of 10⁻⁸ nM or less, 10⁻⁹ nM or less, or 10⁻¹⁰ nM or less.

In one aspect, an anti-TGF-beta 1 antibody binds to a latent TGF-beta 1 forming LLC, and/or a latent TGF-beta 1 forming complex with GARP or LRRC33. In certain embodiments, an anti-TGF-beta 1 antibody binds to cell surface latent TGF-beta 1, which is a latent TGF-beta 1 associated with the extracellular matrix (ECM) on a cell surface. In another aspect, an anti-TGF-beta 1 antibody binds to a latent TGF-beta 1, wherein the LAP region of the latent TGF-beta 1 is not linked to LTBP, forming the small latent complex (SLC). In certain embodiments, SLCs exist in a soluble form. In some embodiments, an anti-TGF-beta 1 antibody binds to latent TGF-beta 1 (cell surface latent TGF-beta 1, LLC, or SLC) with an affinity or binding activity of 10-8 nM or less, 10-9 nM or less, or 10-10 nM or less.

In one aspect, an anti-TGF-beta 1 antibody inhibits activation of a latent TGF-beta 1. The term “activation” of latent TGF-beta 1, as used herein, refers to any process in which mature TGF-beta 1 is released from LAP, which is a component of the latent TGF-beta 1. The activation of latent TGF-beta 1 can be detected, for example, by measuring mature TGF-beta 1 and/or measuring mature TGF-beta 1 activity using various techniques known in the art or described herein. In some embodiments, an anti-TGF-beta 1 antibody inhibits the release of mature TGF-beta 1 from latent TGF-beta 1. As described above, it has been reported that mature TGF-beta 1 is released from latent TGF-beta 1 by activators such as proteases, integrins and other non-protease activators. Non-limited examples of proteases which activate latent TGF-beta 1 include plasmin (PLN), prekallikrein (PLK), matrix metalloproteinase (MMP) 2 and MMP9. In some embodiments, an anti-TGF-beta 1 antibody inhibits protease mediated and/or integrin mediated release of mature TGF-beta 1 from latent TGF-beta 1. As described above, proteases cleave LAP region of latent TGF-beta 1, which causes release of mature TGF-beta 1. In some embodiments, the cleavage sites by PLN and/or PLK locate within a fragment consisting of amino acids 56-59 of LAP polypeptide.

In one aspect, an anti-TGF-beta 1 antibody inhibits protease mediated release of mature TGF-beta 1 from latent TGF-beta 1 without inhibiting protease mediated cleavage of LAP portion of latent TGF-beta 1. In some embodiments, an anti-TGF-beta 1 antibody inhibits protease mediated release of mature TGF-beta 1 from latent TGF-beta 1 and allows a protease to cleave the LAP region while the anti-TGF-beta 1 antibody binds to the LAP region of the latent TGF-beta 1. In some embodiments, an anti-TGF-beta 1 antibody does not block access of a protease to latent TGF-beta 1, especially to the cleavage sites by PLN and/or PLK. In other embodiments, an anti-TGF-beta 1 antibody does not bind to protease cleavage sites of LAP portion of a latent TGF-beta 1, especially the cleavage sites by PLN and/or PLK.

In some embodiments, an anti-TGF-beta 1 antibody that inhibits protease mediated release of mature TGF-beta 1 from latent TGF-beta 1 is an antibody which (i) inhibits cleavage of LAP region mediated by one or more proteases, but (ii) does not inhibit cleavage of LAP region mediated by other proteases. For example, an anti-TGF-beta 1 antibody (1-i) inhibits MMP2 and/or MMP9 mediated release of mature TGF-beta 1 by inhibiting MMP2 and/or MMP9 mediated cleavage of LAP portion of latent TGF-beta 1, and (1-ii) inhibits PLN and/or PLK mediated release of mature TGF-beta 1 without inhibiting PLN and/or PLK mediated cleavage of LAP portion of latent TGF-beta 1. Alternatively, an anti-TGF-beta 1 antibody (2-i) inhibits PLN and/or PLK mediated release of mature TGF-beta 1 by inhibiting PLN and/or PLK mediated cleavage of LAP portion of latent TGF-beta 1, and (2-ii) inhibits MMP2 and/or MMP9 mediated release of mature TGF-beta 1 without inhibiting MMP2 and/or MMP9 mediated cleavage of LAP portion of latent TGF-beta 1. Alternatively, an anti-TGF-beta 1 antibody (3-i) inhibits PLN and/or PLK mediated release of mature TGF-beta 1 without inhibiting PLN and/or PLK mediated cleavage of LAP portion of latent TGF-beta 1, and (3-ii) inhibits MMP2 and/or MMP9 mediated release of mature TGF-beta 1 without inhibiting MMP2 and/or MMP9 mediated cleavage of LAP portion of latent TGF-beta 1.

In some embodiments, antibodies “which inhibit activation of a latent TGF-beta 1” include antibodies that cause at least 5%, 10%, 15%, 20%, 25%, 30%, 35%, or 40% or greater decrease in TGF-beta 1 activation. In other embodiments, antibodies “which inhibit protease mediated release of mature TGF-beta 1 from latent TGF-beta 1” include antibodies that cause at least 5%, 10%, 15%, 20%, 25%, 30%, 35%, or 40% or greater decrease in protease mediated release of mature TGF-beta 1 from latent TGF-beta 1. In further embodiments, antibodies which inhibit protease mediated release of mature TGF-beta 1 from latent TGF-beta 1 “without inhibiting protease mediated cleavage of LAP region of latent TGF-beta 1” include antibodies that cause 50% or less, 45% or less, 40% or less, 35% or less, 30% or less, 25% or less, 20% or less, 15% or less, 10% or less, 5% or less decrease in protease mediated cleavage of LAP region of latent TGF-beta 1.

In some embodiments, an anti-TGF-beta 1 antibody stabilizes the structure of LAP region of the latent TGF-beta 1 without inhibiting the protease mediated cleavage of the LAP region of the latent TGF-beta 1. When an anti-TGF-beta 1 antibody “stabilize” the structure of LAP region, as used herein, the LAP region bounded by the anti-TGF-beta 1 antibody was kept in a certain structure from which mature TGF-beta 1 cannot be released. In further embodiments, latent TGF-beta 1 which is stabilized by an anti-TGF-beta 1 antibody can be activated by integrin. In certain embodiments, the LAP region which is stabilized by an anti-TGF-beta 1 antibody has been either cleaved or not cleaved by a protease. In some embodiments, an anti-TGF-beta 1 antibody stabilizes the structure of LAP region of the latent TGF-beta 1 and allows a protease to cleave the LAP region while the anti-TGF-beta 1 antibody binds to the LAP region of the latent TGF-beta 1. In some embodiments, an anti-TGF-beta 1 antibody stabilizes the structure of LAP region of the latent TGF-beta 1 without blocking access of a protease to latent TGF-beta 1, especially to the cleavage sites by PLN and/or PLK. In other embodiments, an anti-TGF-beta 1 antibody stabilizes the structure of LAP region of the latent TGF-beta 1 without blocking access of a protease to latent TGF-beta 1, especially to the cleavage sites by MEMP2 and/or MEMP9.

In one aspect, an anti-TGF-beta 1 antibody does not bind to mature TGF-beta 1. In some embodiments, an anti-TGF-beta 1 antibody binds to latent TGF-beta 1 with higher affinity or binding activity than mature TGF-beta 1. In certain embodiments, the antibodies of the present invention bind to latent TGF-beta 1 with at least 2, 3, 5, 10, 15, 20, 25, 30, 35, 40, 45, 50, 55, 60, 65, 70, 75, 80, 85, 90, 95, 100, 200, 400, 1000, 10000, or more times higher affinity or binding activity than to mature TGF-beta 1.

In one aspect, an anti-TGF-beta 1 antibody does not or does partially inhibit integrin mediated TGF-beta 1 activation, i.e, integrin mediated release of mature TGF-beta 1 from latent TGF-beta 1. In some embodiments, antibodies “which does not or does partially inhibit integrin mediated TGF-beta 1 activation” include antibodies that cause 50% or less, 45% or less, 40% or less, 35% or less, 30% or less, 25% or less, 20% or less, 15% or less, 10% or less, 5% or less decrease in integrin mediated TGF-beta 1 activation, i.e, integrin mediated release of mature TGF-beta 1 from latent TGF-beta 1.

In some embodiments, an anti-TGF-beta 1 antibody of the present invention:
binds to latent TGF-beta 1;
binds to latent TGF-beta 1 forming SLC;
binds to latent TGF-beta 1 forming LLC;
binds to latent TGF-beta 1 forming complex with GARP or LRRC33;
binds to cell surface latent TGF-beta 1;
binds to LAP region of latent TGF-beta 1;
binds to LAP;
binds to latent TGF-beta 1 with an affinity or binding activity of 10⁻⁸ nM or less, 10⁻⁹ nM or less, or 10⁻¹⁰ nM or less;
inhibits protease mediated release of mature TGF-beta 1 from latent TGF-beta 1;
does not inhibit protease mediated cleavage of LAP region of latent TGF-beta 1; and/or
does not or does partially inhibit integrin mediated release of mature TGF-beta 1 from latent TGF-beta 1.

In further embodiments, the anti-TGF-beta 1 antibody of the present invention is:
a monoclonal antibody;
a human, humanized or chimeric antibody;
a full length of IgG antibody; and/or
an antibody fragment.

In one aspect, the invention provides an anti-TGF-beta 1 antibody comprising at least one, two, three, four, five, or six HVRs selected from (a) HVR-H1 comprising the amino acid sequence of SEQ ID NO: 5; (b) HVR-H2 comprising the amino acid sequence of SEQ ID NO: 6; (c) HVR-H3 comprising the amino acid sequence of SEQ ID NO: 7; (d) HVR-L1 comprising the amino acid sequence of SEQ ID NO: 8; (e) HVR-L2 comprising the amino acid sequence of SEQ ID NO: 9; and (f) HVR-L3 comprising the amino acid sequence of SEQ ID NO: 10.

In one aspect, the invention provides an anti-TGF-beta 1 antibody comprising at least one, two, three, four, five, or six HVRs selected from (a) HVR-H1 comprising the amino acid sequence of SEQ ID NO: 11; (b) HVR-H2 comprising the amino acid sequence of SEQ ID NO: 12; (c) HVR-H3 comprising the amino acid sequence of SEQ ID NO: 13; (d) HVR-L1 comprising the amino acid sequence of SEQ ID NO: 14; (e) HVR-L2 comprising the amino acid sequence of SEQ ID NO: 15; and (f) HVR-L3 comprising the amino acid sequence of SEQ ID NO: 16.

In one aspect, the invention provides an anti-TGF-beta 1 antibody comprising at least one, two, three, four, five, or six HVRs selected from (a) HVR-H1 comprising the amino acid sequence of SEQ ID NO: 17; (b) HVR-H2 comprising the amino acid sequence of SEQ ID NO: 18; (c) HVR-H3 comprising the amino acid sequence of SEQ ID NO: 19; (d) HVR-L1 comprising the amino acid sequence of SEQ ID NO: 20; (e) HVR-L2 comprising the amino acid sequence of SEQ ID NO: 21; and (f) HVR-L3 comprising the amino acid sequence of SEQ ID NO: 22.

In any of the above embodiments, an anti-TGF-beta 1 antibody is humanized. In one embodiment, an anti-TGF-beta 1 antibody comprises HVRs as in any of the above embodiments, and further comprises an acceptor human framework, e.g. a human immunoglobulin framework or a human consensus framework.

In another aspect, an anti-TGF-beta 1 antibody is provided, wherein the antibody comprises a VH as in any of the embodiments provided above, and a VL as in any of the embodiments provided above. In one embodiment, the antibody comprises the VH and VL sequences in SEQ ID NO: 23 and SEQ ID NO: 24, respectively, including post-translational modifications of those sequences. Post-translational modifications include but are not limited to a modification of glutamine or glutamate in N-terminal of heavy chain or light chain to pyroglutamic acid by pyroglutamylation.

In another aspect, an anti-TGF-beta 1 antibody is provided, wherein the antibody comprises a VH as in any of the embodiments provided above, and a VL as in any of the embodiments provided above. In one embodiment, the antibody comprises the VH and VL sequences in SEQ ID NO: 25 and SEQ ID NO: 26, respectively, including post-translational modifications of those sequences. Post-translational modifications include but are not limited to a modification of glutamine or glutamate in N-terminal of heavy chain or light chain to pyroglutamic acid by pyroglutamylation.

In another aspect, an anti-TGF-beta 1 antibody is provided, wherein the antibody comprises a VH as in any of the embodiments provided above, and a VL as in any of the embodiments provided above. In one embodiment, the antibody comprises the VH and VL sequences in SEQ ID NO: 27 and SEQ ID NO: 28, respectively, including post-translational modifications of those sequences. Post-translational modifications include but are not limited to a modification of glutamine or glutamate in N-terminal of heavy chain or light chain to pyroglutamic acid by pyroglutamylation.

In a further aspect, the invention provides an antibody that binds to the same epitope as an anti-TGF-beta 1 antibody provided herein. For example, in certain embodiments, an antibody is provided that binds to the same epitope as
(1) an anti-TGF-beta 1 antibody comprising (a) HVR-H1 comprising the amino acid sequence of SEQ ID NO: 5; (b) HVR-H2 comprising the amino acid sequence of SEQ ID NO: 6; (c) HVR-H3 comprising the amino acid sequence of SEQ ID NO: 7; (d) HVR-L1 comprising the amino acid sequence of SEQ ID NO: 8; (e) HVR-L2 comprising the amino acid sequence of SEQ ID NO: 9; and (f) HVR-L3 comprising the amino acid sequence of SEQ ID NO: 10;
(2) an anti-TGF-beta 1 antibody comprising (a) HVR-H1 comprising the amino acid sequence of SEQ ID NO: 11; (b) HVR-H2 comprising the amino acid sequence of SEQ ID NO: 12; (c) HVR-H3 comprising the amino acid sequence of SEQ ID NO: 13; (d) HVR-L1 comprising the amino acid sequence of SEQ ID NO: 14; (e) HVR-L2 comprising the amino acid sequence of SEQ ID NO: 15; and (f) HVR-L3 comprising the amino acid sequence of SEQ ID NO: 16;
(3) an anti-TGF-beta 1 antibody comprising (a) HVR-H1 comprising the amino acid sequence of SEQ ID NO: 17; (b) HVR-H2 comprising the amino acid sequence of SEQ ID NO: 18; (c) HVR-H3 comprising the amino acid sequence of SEQ ID NO: 19; (d) HVR-L1 comprising the amino acid sequence of SEQ ID NO: 20; (e) HVR-L2 comprising the amino acid sequence of SEQ ID NO: 21; and (f) HVR-L3 comprising the amino acid sequence of SEQ ID NO: 22;

In a further aspect, the invention provides an antibody that binds to TGF-beta 1 of human, monkey, mouse, and/or rat. In certain embodiments, the invention provides an antibody that binds to TGF-beta 1 of human and mouse. In certain embodiments, the invention provides an antibody which binds to latent TGF-beta 1 forming SLC of human and mouse. In certain embodiments, the invention provides an antibody which binds to latent TGF-beta 1 forming LLC of human and mouse. In certain embodiments, the invention provides an antibody which binds to latent TGF-beta 1 forming LLC of human and mouse. In certain embodiments, the invention provides an antibody which binds to latent TGF-beta 1 forming complex with GARP or LRRC33 of human and mouse. In certain embodiments, the invention provides an antibody which binds to cell surface latent TGF-beta 1 of human and mouse.

In a further aspect, the invention provides an antibody that binds to the same epitope as any one of the anti-TGF-beta 1 antibodies provided herein. The epitope may exist on TGF-beta 1 of human, monkey, mouse and/or rat. For example, in certain embodiments, the invention provides an antibody that binds the same epitope as a reference antibody, wherein the reference antibody is:
(1) an anti-TGF-beta 1 antibody comprising (a) HVR-H1 comprising the amino acid sequence of SEQ ID NO: 5; (b) HVR-H2 comprising the amino acid sequence of SEQ ID NO: 6; (c) HVR-H3 comprising the amino acid sequence of SEQ ID NO: 7; (d) HVR-L1 comprising the amino acid sequence of SEQ ID NO: 8; (e) HVR-L2 comprising the amino acid sequence of SEQ ID NO: 9; and (f) HVR-L3 comprising the amino acid sequence of SEQ ID NO: 10;
(2) an anti-TGF-beta 1 antibody comprising (a) HVR-H1 comprising the amino acid sequence of SEQ ID NO: 11; (b) HVR-H2 comprising the amino acid sequence of SEQ ID NO: 12; (c) HVR-H3 comprising the amino acid sequence of SEQ ID NO: 13; (d) HVR-L1 comprising the amino acid sequence of SEQ ID NO: 14; (e) HVR-L2 comprising the amino acid sequence of SEQ ID NO: 15; and (f) HVR-L3 comprising the amino acid sequence of SEQ ID NO: 16;
(3) an anti-TGF-beta 1 antibody comprising (a) HVR-H1 comprising the amino acid sequence of SEQ ID NO: 17; (b) HVR-H2 comprising the amino acid sequence of SEQ ID NO: 18; (c) HVR-H3 comprising the amino acid sequence of SEQ ID NO: 19; (d) HVR-L1 comprising the amino acid sequence of SEQ ID NO: 20; (e) HVR-L2 comprising the amino acid sequence of SEQ ID NO: 21; and (f) HVR-L3 comprising the amino acid sequence of SEQ ID NO: 22.

In a further aspect, the invention provides an antibody that competes with an anti-TGF-beta 1 antibody provided herein for binding to TGF-beta 1 of human, monkey, mouse and/or rat. For example, in certain embodiments, an antibody is provided that competes for binding TGF-beta 1 of human, monkey, mouse and/or rat with
(1) an anti-TGF-beta 1 antibody comprising (a) HVR-H1 comprising the amino acid sequence of SEQ ID NO: 5; (b) HVR-H2 comprising the amino acid sequence of SEQ ID NO: 6; (c) HVR-H3 comprising the amino acid sequence of SEQ ID NO: 7; (d) HVR-L1 comprising the amino acid sequence of SEQ ID NO: 8; (e) HVR-L2 comprising the amino acid sequence of SEQ ID NO: 9; and (f) HVR-L3 comprising the amino acid sequence of SEQ ID NO: 10;
(2) an anti-TGF-beta 1 antibody comprising (a) HVR-H1 comprising the amino acid sequence of SEQ ID NO: 11; (b) HVR-H2 comprising the amino acid sequence of SEQ ID NO: 12; (c) HVR-H3 comprising the amino acid sequence of SEQ ID NO: 13; (d) HVR-L1 comprising the amino acid sequence of SEQ ID NO: 14; (e) HVR-L2 comprising the amino acid sequence of SEQ ID NO: 15; and (f) HVR-L3 comprising the amino acid sequence of SEQ ID NO: 16;
(3) an anti-TGF-beta 1 antibody comprising (a) HVR-H1 comprising the amino acid sequence of SEQ ID NO: 17; (b) HVR-H2 comprising the amino acid sequence of SEQ ID NO: 18; (c) HVR-H3 comprising the amino acid sequence of SEQ ID NO: 19; (d) HVR-L1 comprising the amino acid sequence of SEQ ID NO: 20; (e) HVR-L2 comprising the amino acid sequence of SEQ ID NO: 21; and (f) HVR-L3 comprising the amino acid sequence of SEQ ID NO: 22;

In a further aspect of the invention, an anti-TGF-beta 1 antibody according to any of the above embodiments is a monoclonal antibody, including a chimeric, humanized or human antibody. In one embodiment, an anti-TGF-beta 1 antibody is an antibody fragment, e.g., a Fv, Fab, Fab′, scFv, diabody, or F(ab′)2 fragment. In another embodiment, the antibody is a full length antibody, e.g., an intact IgG1, IgG2, IgG3 or IgG4 antibody or other antibody class or isotype as defined herein. In a further aspect, an anti-TGF-beta 1 antibody also includes any antigen binding molecule which comprises a variable heavy chain and/or variable light chain structure of immunoglobulin.

In a further aspect, an anti-TGF-beta 1 antibody according to any of the above embodiments may incorporate any of the features, singly or in combination, as described in Sections 1-7 below:

### 1. Antibody Affinity

In certain embodiments, an antibody provided herein has a dissociation constant (Kd) of 1 micro M or less, 100 nM or less, 10 nM or less, 1 nM or less, 0.1 nM or less, 0.01 nM or less, or 0.001 nM or less (e.g. 10-8 M or less, e.g. from 10⁻⁸ M to 10⁻¹³ M, e.g., from 10⁻⁹ M to 10⁻¹³ M).

In one embodiment, Kd is measured by a radiolabeled antigen binding assay (RIA). In one embodiment, an RIA is performed with the Fab version of an antibody of interest and its antigen. For example, solution binding affinity of Fabs for antigen is measured by equilibrating Fab with a minimal concentration of (125I)-labeled antigen in the presence of a titration series of unlabeled antigen, then capturing bound antigen with an anti-Fab antibody-coated plate (see, e.g., Chen et al., J. Mol. Biol. 293:865-881(1999)). To establish conditions for the assay, MICROTITER (registered trademark) multi-well plates (Thermo Scientific) are coated overnight with 5 micro g/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23 degrees C.). In a non-adsorbent plate (Nunc #269620), 100 pM or 26 pM [125I]-antigen are mixed with serial dilutions of a Fab of interest (e.g., consistent with assessment of the anti-VEGF antibody, Fab-12, in Presta et al., Cancer Res. 57:4593-4599 (1997)). The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% polysorbate 20 (TWEEN-20 (registered trademark)) in PBS. When the plates have dried, 150 micro 1/well of scintillant (MICROSCINT-20 ™; Packard) is added, and the plates are counted on a TOPCOUNT™ gamma counter (Packard) for ten minutes. Concentrations of each Fab that give less than or equal to 20% of maximal binding are chosen for use in competitive binding assays.

According to another embodiment, Kd is measured using a BIACORE (registered trademark) surface plasmon resonance assay. For example, an assay using a BIACORE (registered trademark)-2000 or a BIACORE(registered trademark)-3000 (BIAcore, Inc., Piscataway, NJ) is performed at 25 degrees C. with immobilized antigen CM5 chips at ˜10 response units (RU). In one embodiment, carboxymethylated dextran biosensor chips (CM5, BIACORE, Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 micro g/ml (˜0.2 micro M) before injection at a flow rate of 5 micro 1/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% polysorbate 20 (TWEEN-20™) surfactant (PBST) at 25 degrees C. at a flow rate of approximately 25 micro 1/min. Association rates (ka or kon) and dissociation rates (kd or koff) are calculated using a simple one-to-one Langmuir binding model (BIACORE (registered trademark) Evaluation Software version 3.2) by simultaneously fitting the association and dissociation sensorgrams. The equilibrium dissociation constant (Kd) is calculated as the ratio koff/kon. See, e.g., Chen et al., J. Mol. Biol. 293:865-881 (1999). If the on-rate exceeds 106 M−1 s−1 by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25 degrees C. of a 20 nM anti-antigen antibody (Fab form) in PBS, pH 7.2, in the presence of increasing concentrations of antigen as measured in a spectrometer, such as a stop-flow equipped spectrophotometer (Aviv Instruments) or a 8000-series SLM-AMINCO™ spectrophotometer (ThermoSpectronic) with a stirred cuvette. In certain embodiments, an antibody for TGF-beta 1 has a Kd of 1 micro M or less, 100 nM or less, 10 nM or less, 1 nM or less, 0.1 nM or less, 0.01 nM or less, or 0.001 nM or less, with a koff of 5×10-2 s−1 or less, 1×10-2 s−1 or less, 5×10-3 s−1 or less, 1×10-3 s−1 or less, 5×10-4 s−1 or less, 1×10-4 s−1 or less, 5×10-5 s−1 or less, 1×10-5 s−1 or less, 5×10-6 s−1 or less, 1×10-6 s−1 or less, 5×10-7 s−1 or less, or 1×10-7 s−1 or less.

### 2. Antibody Binding Activity

A “binding activity” of an antibody for TGF-beta 1 refers to the strength of the sum total of noncovalent interactions between binding sites of a molecule (e.g., an antibody) and its binding partner (e.g., an antigen). Unless indicated otherwise, as used herein, “binding activity” is not strictly restricted to 1:1 interaction between members of a binding pair (e.g., antibody and antigen), but can be affected by the avidity of the interaction between members of a binding pair (e.g., antibody and antigen). The binding activity of a molecule X for its partner Y can generally be represented by the dissociation constant (Kd). Binding activity can be measured by common methods known in the art, including those described herein. The term “affinity” may be used interchangeably with “binding activity”.

The “binding activity” of an antibody for TGF-beta 1 can be expressed in terms of the Kd of the antibody. In certain embodiments, an antibody provided herein has a dissociation constant (Kd) of 1 micro M or less, 100 nM or less, 10 nM or less, 1 nM or less, 0.1 nM or less, 0.01 nM or less, or 0.001 nM or less (e.g. 10-8 M or less, e.g. from 10-8 M to 10-13 M, e.g., from 10-9 M to 10-13 M). In one embodiment, Kd is measured by a radiolabeled antigen binding assay (RIA) as described above. According to another embodiment, Kd is measured using a BIACORE (registered trademark) surface plasmon resonance assay as described above. The binding activity (Kd) can be determined from association rate constant (ka or kon) and dissociation rate constant (kd or koff) using 1:1 binding model. It is clear to the skilled person that measuring process somehow influences the intrinsic binding activity of the implied molecules for example by artefacts related to the coating on the biosensor of one molecule. Also, if one molecule contains more than one recognition sites for the other molecule, the measured Kd may be affected by the avidity of the interaction by the two molecules. In certain embodiments, an antibody for TGF-beta 1 has a Kd of 1 micro M or less, 100 nM or less, 10 nM or less, 1 nM or less, 0.1 nM or less, 0.01 nM or less, or 0.001 nM or less, with a koff of 5×10-2 s−1 or less, 1×10-2 s−1 or less, 5×10-3 s−1 or less, 1×10-3 s−1 or less, 5×10-4 s−1 or less, 1×10-4 s−1 or less, 5×10-5 s−1 or less, 1×10-5 s−1 or less, 5×10-6 s−1 or less, 1×10-6 s−1 or less, 5×10-7 s−1 or less, or 1×10-7 s−1 or less.

### 3. Antibody Fragments

In certain embodiments, an antibody provided herein is an antibody fragment. Antibody fragments include, but are not limited to, Fab, Fab′, Fab′-SH, F(ab′)2, Fv, and scFv fragments, and other fragments described below. For a review of certain antibody fragments, see Hudson et al. Nat. Med. 9:129-134 (2003). For a review of scFv fragments, see, e.g., Pluckthun, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458. For discussion of Fab and F(ab′)2 fragments comprising salvage receptor binding epitope residues and having increased in vivo half-life, see U.S. Pat. No. 5,869,046.

Diabodies are antibody fragments with two antigen-binding sites that may be bivalent or bispecific. See, for example, EP 404,097; WO 1993/01161; Hudson et al., Nat. Med. 9:129-134 (2003); and Hollinger et al., Proc. Natl. Acad. Sci. USA 90: 6444-6448 (1993). Triabodies and tetrabodies are also described in Hudson et al., Nat. Med. 9:129-134 (2003).

Single-domain antibodies are antibody fragments comprising all or a portion of the heavy chain variable domain or all or a portion of the light chain variable domain of an antibody. In certain embodiments, a single-domain antibody is a human single-domain antibody (Domantis, Inc., Waltham, MA; see, e.g., U.S. Pat. No. 6,248,516 B1).

Antibody fragments can be made by various techniques, including but not limited to proteolytic digestion of an intact antibody as well as production by recombinant host cells (e.g. E. coli or phage), as described herein.

The present invention also relates to antigen-binding molecules which bind to TGF-beta 1, which includes, but are not limited to, for example, minibodies (low molecular weight antibodies), and scaffold proteins. In the present invention, any scaffold protein is acceptable as long as it is a peptide that has a stable three-dimensional structure and is capable of binding to at least an antigen. Such peptides include, for example, fragments of antibody variable regions, fibronectin, protein A domain, LDL receptor A domain, lipocalin, and other molecules described in Nygren et al. (Current Opinion in Structural Biology, (1997) 7:463-469; Journal of Immunol Methods, (2004) 290:3-28), Binz et al. (Nature Biotech. (2005) 23:1257-1266), and Hosse et al. (Protein Science, (2006) 15:14-27). When referring to such an antibody, e.g., “anti-TGF-beta 1 antibody” should be replaced with “anti-TGF-beta 1 antigen-binding molecule” in the context of the present specification.

### 4. Chimeric and Humanized Antibodies

In certain embodiments, an antibody provided herein is a chimeric antibody. Certain chimeric antibodies are described, e.g., in U.S. Pat. No. 4,816,567; and Morrison et al., Proc. Natl. Acad. Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In certain embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing specificity determining region (SDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

### 5. Human Antibodies

In certain embodiments, an antibody provided herein is a human antibody. Human antibodies can be produced using various techniques known in the art. Human antibodies are described generally in van Dijk and van de Winkel, Curr. Opin. Pharmacol. 5: 368-74 (2001) and Lonberg, Curr. Opin. Immunol. 20:450-459 (2008).

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE™ technology; U.S. Pat. No. 5,770,429 describing HUMAB (registered trademark) technology; U.S. Pat. No. 7,041,870 describing K-M MOUSE (registered trademark) technology, and U.S. Patent Application Publication No. US 2007/0061900, describing VELOCIMOUSE (registered trademark) technology). Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991).) Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3):185-91 (2005).

Human antibodies may also be generated by isolating Fv clone variable domain sequences selected from human-derived phage display libraries. Such variable domain sequences may then be combined with a desired human constant domain. Techniques for selecting human antibodies from antibody libraries are described below.

### 6. Library-Derived Antibodies

Antibodies of the invention may be isolated by screening combinatorial libraries for antibodies with the desired activity or activities. For example, a variety of methods are known in the art for generating phage display libraries and screening such libraries for antibodies possessing the desired binding characteristics. Such methods are reviewed, e.g., in Hoogenboom et al. in Methods in Molecular Biology 178:1-37 (O'Brien et al., ed., Human Press, Totowa, NJ, 2001) and further described, e.g., in the McCafferty et al., Nature 348:552-554; Clackson et al., Nature 352: 624-628 (1991); Marks et al., J. Mol. Biol. 222: 581-597 (1992); Marks and Bradbury, in Methods in Molecular Biology 248:161-175 (Lo, ed., Human Press, Totowa, NJ, 2003); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132(2004).

In certain phage display methods, repertoires of VH and VL genes are separately cloned by polymerase chain reaction (PCR) and recombined randomly in phage libraries, which can then be screened for antigen-binding phage as described in Winter et al., Ann. Rev. Immunol., 12: 433-455 (1994). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self antigens without any immunization as described by Griffiths et al., EMBO J, 12: 725-734 (1993). Finally, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter, J. Mol. Biol., 227: 381-388 (1992). Patent publications describing human antibody phage libraries include, for example: U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360.

Antibodies or antibody fragments isolated from human antibody libraries are considered human antibodies or human antibody fragments herein.

### 7. Multispecific Antibodies

In certain embodiments, an antibody provided herein is a multispecific antibody, e.g. a bispecific antibody. Multispecific antibodies are monoclonal antibodies that have binding specificities for at least two different sites. In certain embodiments, one of the binding specificities is for TGF-beta 1 and the other is for any other antigen. In certain embodiments, bispecific antibodies may bind to two different epitopes of TGF-beta 1. Bispecific antibodies may also be used to localize cytotoxic agents to cells which express TGF-beta 1. Bispecific antibodies can be prepared as full length antibodies or antibody fragments.

Techniques for making multispecific antibodies include, but are not limited to, recombinant co-expression of two immunoglobulin heavy chain-light chain pairs having different specificities (see Milstein and Cuello, Nature 305: 537 (1983)), WO 93/08829, and Traunecker et al., EMBO J. 10: 3655 (1991)), and “knob-in-hole” engineering (see, e.g., U.S. Pat. No. 5,731,168). Multi-specific antibodies may also be made by engineering electrostatic steering effects for making antibody Fc-heterodimeric molecules (WO 2009/089004A1); cross-linking two or more antibodies or fragments (see, e.g., U.S. Pat. No. 4,676,980, and Brennan et al., Science, 229: 81 (1985)); using leucine zippers to produce bi-specific antibodies (see, e.g., Kostelny et al., J. Immunol., 148(5):1547-1553 (1992)); using “diabody” technology for making bispecific antibody fragments (see, e.g., Hollinger et al., Proc. Natl. Acad. Sci. USA, 90:6444-6448 (1993)); and using single-chain Fv (scFv) dimers (see, e.g. Gruber et al., J. Immunol., 152:5368 (1994)); and preparing trispecific antibodies as described, e.g., in Tutt et al. J. Immunol. 147: 60 (1991).

Engineered antibodies with three or more functional antigen binding sites, including “Octopus antibodies,” are also included herein (see, e.g. US 2006/0025576A1).

The antibody or fragment herein also includes a “Dual Acting Fab” or “DAF” comprising an antigen binding site that binds to TGF-beta 1 as well as another, different antigen (see, US 2008/0069820, for example).

### 8. Antibody Variants

In certain embodiments, amino acid sequence variants of the antibodies provided herein are contemplated. For example, it may be desirable to improve the binding affinity and/or other biological properties of the antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

As mentioned above, TGF-beta is a member of the TGF-beta superfamily of cytokines, including myostatin and separate TGF-beta isoforms such as TGF-beta 1, TGF-beta 2, and TGF-beta 3. Thus, the present invention also relates to an antibody that binds to TGF-beta 2 or TGF-beta 3, or a member of the TGF-beta superfamily such as myostatin. When referring to such an antibody, e.g., “anti-TGF-beta 1 antibody” should be replaced with “anti-myostatin antibody”, “anti-TGF-beta 2 antibody”, “anti-TGF-beta 3 antibody”, “an antibody against the member of the TGF-beta superfamily” in the context of the present specification.

### a) Substitution, Insertion, and Deletion Variants

In certain embodiments, antibody variants having one or more amino acid substitutions are provided. Sites of interest for substitutional mutagenesis include the HVRs and FRs. Conservative substitutions are shown in Table 1 under the heading of “preferred substitutions.” More substantial changes are provided in Table 1 under the heading of “exemplary substitutions,” and as further described below in reference to amino acid side chain classes. Amino acid substitutions may be introduced into an antibody of interest and the products screened for a desired activity, e.g., retained/improved antigen binding, decreased immunogenicity, or improved ADCC or CDC.

TABLE 1

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.
Non-conservative substitutions will entail exchanging a member of one of these classes for another class.

One type of substitutional variant involves substituting one or more hypervariable region residues of a parent antibody (e.g. a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques such as those described herein. Briefly, one or more HVR residues are mutated and the variant antibodies displayed on phage and screened for a particular biological activity (e.g. binding affinity).

Alterations (e.g., substitutions) may be made in HVRs, e.g., to improve antibody affinity. Such alterations may be made in HVR “hotspots,” i.e., residues encoded by codons that undergo mutation at high frequency during the somatic maturation process (see, e.g., Chowdhury, Methods Mol. Biol. 207:179-196 (2008)), and/or residues that contact antigen, with the resulting variant VH or VL being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, NJ, (2001).) In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves HVR-directed approaches, in which several HVR residues (e.g., 4-6 residues at a time) are randomized. HVR residues involved in antigen binding may be specifically identified, e.g., using alanine scanning mutagenesis or modeling. CDR-H3 and CDR-L3 in particular are often targeted.

In certain embodiments, substitutions, insertions, or deletions may occur within one or more HVRs so long as such alterations do not substantially reduce the ability of the antibody to bind antigen. For example, conservative alterations (e.g., conservative substitutions as provided herein) that do not substantially reduce binding affinity may be made in HVRs. Such alterations may, for example, be outside of antigen contacting residues in the HVRs. In certain embodiments of the variant VH and VL sequences provided above, each HVR either is unaltered, or contains no more than one, two or three amino acid substitutions.

A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as arg, asp, his, lys, and glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex may be analyzed to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion of an enzyme (e.g. for ADEPT) or a polypeptide which increases the plasma half-life of the antibody to the N- or C-terminus of the antibody.

### b) Glycosylation Variants

In certain embodiments, an antibody provided herein is altered to increase or decrease the extent to which the antibody is glycosylated. Addition or deletion of glycosylation sites to an antibody may be conveniently accomplished by altering the amino acid sequence such that one or more glycosylation sites is created or removed.

Where the antibody comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an antibody of the invention may be made in order to create antibody variants with certain improved properties.

In one embodiment, antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e. g. complex, hybrid and high mannose structures) as measured by MALDI-TOF mass spectrometry, as described in WO 2008/077546, for example. Asn297 refers to the asparagine residue located at about position 297 in the Fc region (EU numbering of Fc region residues); however, Asn297 may also be located about +/−3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87: 614 (2004). Examples of cell lines capable of producing defucosylated antibodies include Lec13 CHO cells deficient in protein fucosylation (Ripka et al. Arch. Biochem. Biophys. 249:533-545 (1986); US Pat Appl No US 2003/0157108 A1, Presta, L; and WO 2004/056312 A1, Adams et al., especially at Example 11), and knockout cell lines, such as alpha-1,6-fucosyltransferase gene, FUT8, knockout CHO cells (see, e.g., Yamane-Ohnuki et al. Biotech. Bioeng. 87: 614 (2004); Kanda, Y. et al., Biotechnol. Bioeng., 94(4):680-688 (2006); and WO2003/085107).

Antibodies variants are further provided with bisected oligosaccharides, e.g., in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878 (Jean-Mairet et al.); U.S. Pat. No. 6,602,684 (Umana et al.); and US 2005/0123546 (Umana et al.). Antibody variants with at least one galactose residue in the oligosaccharide attached to the Fc region are also provided. Such antibody variants may have improved CDC function. Such antibody variants are described, e.g., in WO 1997/30087 (Patel et al.); WO 1998/58964 (Raju, S.); and WO 1999/22764 (Raju, S.).

### c) Fc Region Variants

In certain embodiments, one or more amino acid modifications may be introduced into the Fc region of an antibody provided herein, thereby generating an Fc region variant. The Fc region variant may comprise a human Fc region sequence (e.g., a human IgG1, IgG2, IgG3 or IgG4 Fc region) comprising an amino acid modification (e.g. a substitution) at one or more amino acid positions.

In certain embodiments, the invention contemplates an antibody variant that possesses some but not all effector functions, which make it a desirable candidate for applications in which the half life of the antibody in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks Fc gamma R binding (hence likely lacking ADCC activity), but retains FcRn binding ability. The primary cells for mediating ADCC, NK cells, express Fc gamma RIII only, whereas monocytes express Fc gamma RI, Fc gamma RII and Fc gamma RIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g. Hellstrom, I. et al. Proc. Nat'l Acad. Sci. USA 83:7059-7063 (1986)) and Hellstrom, I et al., Proc. Nat'l Acad. Sci. USA 82:1499-1502 (1985); 5,821,337 (see Bruggemann, M. et al., J. Exp. Med. 166:1351-1361 (1987)). Alternatively, non-radioactive assays methods may be employed (see, for example, ACT1™ non-radioactive cytotoxicity assay for flow cytometry (CellTechnology, Inc. Mountain View, CA; and CytoTox 96 (registered trademark) non-radioactive cytotoxicity assay (Promega, Madison, WI). Useful effector cells for such assays include peripheral blood mononuclear cells (PBMC) and Natural Killer (NK) cells. Alternatively, or additionally, ADCC activity of the molecule of interest may be assessed in vivo, e.g., in a animal model such as that disclosed in Clynes et al. Proc. Nat'l Acad. Sci. USA 95:652-656 (1998). C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half life determinations can also be performed using methods known in the art (see, e.g., Petkova, S. B. et al., Int'l. Immunol. 18(12):1759-1769 (2006)).

Antibodies with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Certain antibody variants with increased or decreased binding to FcRs are described. (See, e.g., U.S. Pat. No. 6,737,056; WO 2004/056312, and Shields et al., J. Biol. Chem. 9(2): 6591-6604 (2001).)

In certain embodiments, an antibody variant comprises an Fc region with one or more amino acid substitutions which improve ADCC, e.g., substitutions at positions 298, 333, and/or 334 of the Fc region (EU numbering of residues).

In some embodiments, alterations are made in the Fc region that result in altered (i.e., either increased or decreased) C1q binding and/or Complement Dependent Cytotoxicity (CDC), e.g., as described in U.S. Pat. No. 6,194,551, WO 99/51642, and Idusogie et al. J. Immunol. 164: 4178-4184 (2000).

Antibodies with increased half lives and increased binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934A1 (Hinton et al.). Those antibodies comprise an Fc region with one or more substitutions therein which increase binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826).

See also Duncan & Winter, Nature 322:738-40 (1988); U.S. Pat. Nos. 5,648,260; 5,624,821; and WO 94/29351 concerning other examples of Fc region variants.

### d) Cysteine Engineered Antibody Variants

In certain embodiments, it may be desirable to create cysteine engineered antibodies, e.g., “thioMAbs,” in which one or more residues of an antibody are substituted with cysteine residues. In particular embodiments, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. In certain embodiments, any one or more of the following residues may be substituted with cysteine: V205 (Kabat numbering) of the light chain; A118 (EU numbering) of the heavy chain; and S400 (EU numbering) of the heavy chain Fc region. Cysteine engineered antibodies may be generated as described, e.g., in U.S. Pat. No. 7,521,541.

### e) Antibody Derivatives

In certain embodiments, an antibody provided herein may be further modified to contain additional nonproteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, polypropylene glycol homopolymers, polypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer are attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

In another embodiment, conjugates of an antibody and nonproteinaceous moiety that may be selectively heated by exposure to radiation are provided. In one embodiment, the nonproteinaceous moiety is a carbon nanotube (Kam et al., Proc. Natl. Acad. Sci. USA 102: 11600-11605 (2005)). The radiation may be of any wavelength, and includes, but is not limited to, wavelengths that do not harm ordinary cells, but which heat the nonproteinaceous moiety to a temperature at which cells proximal to the antibody-nonproteinaceous moiety are killed.

### B. Recombinant Methods and Compositions

Antibodies may be produced using recombinant methods and compositions, e.g., as described in U.S. Pat. No. 4,816,567. In one embodiment, isolated nucleic acid encoding an anti-TGF-beta 1 antibody described herein is provided. Such nucleic acid may encode an amino acid sequence comprising the VL and/or an amino acid sequence comprising the VH of the antibody (e.g., the light and/or heavy chains of the antibody). In a further embodiment, one or more vectors (e.g., expression vectors) comprising such nucleic acid are provided. In a further embodiment, a host cell comprising such nucleic acid is provided. In one such embodiment, a host cell comprises (e.g., has been transformed with): (1) a vector comprising a nucleic acid that encodes an amino acid sequence comprising the VL of the antibody and an amino acid sequence comprising the VH of the antibody, or (2) a first vector comprising a nucleic acid that encodes an amino acid sequence comprising the VL of the antibody and a second vector comprising a nucleic acid that encodes an amino acid sequence comprising the VH of the antibody. In one embodiment, the host cell is eukaryotic, e.g. a Chinese Hamster Ovary (CHO) cell or lymphoid cell (e.g., Y0, NS0, Sp2/0 cell). In one embodiment, a method of making an anti-TGF-beta 1 antibody is provided, wherein the method comprises culturing a host cell comprising a nucleic acid encoding the antibody, as provided above, under conditions suitable for expression of the antibody, and optionally recovering the antibody from the host cell (or host cell culture medium).

For recombinant production of an anti-TGF-beta 1 antibody, nucleic acid encoding an antibody, e.g., as described above, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B. K. C. Lo, ed., Humana Press, Totowa, NJ, 2003), pp. 245-254, describing expression of antibody fragments in E. coli.) After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See Gerngross, Nat. Biotech. 22:1409-1414 (2004), and Li et al., Nat. Biotech. 24:210-215 (2006).

Suitable host cells for the expression of glycosylated antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK); buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR-CHO cells (Urlaub et al., Proc. Natl. Acad. Sci. USA 77:4216 (1980)); and myeloma cell lines such as Y0, NS0 and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, NJ), pp. 255-268 (2003).

### C. Assays

Anti-TGF-beta 1 antibodies provided herein may be identified, screened for, or characterized for their physical/chemical properties and/or biological activities by various assays known in the art.

### 1. Binding Assays and Other Assays

In one aspect, an antibody of the invention is tested for its antigen binding activity, e.g., by known methods such as ELISA, Western blot, surface plasmon resonance (e.g. BIACORE(registered trademark)) or a similar technique (e.g. KinExa or OCTET(registered trademark)), etc.

In another aspect, competition assays may be used to identify an antibody that competes with any anti TGF-beta 1 antibodies described herein, preferably TBA0946, TBA0947 or TBA1172 for binding to TGF-beta 1. In certain embodiments, such a competing antibody binds to the same epitope (e.g., a linear or a conformational epitope) that is bound by any anti TGF-beta 1 antibodies described herein, preferably TBA0946, TBA0947 or TBA1172. Detailed exemplary methods for mapping an epitope to which an antibody binds are provided in Morris (1996) “Epitope Mapping Protocols,” in Methods in Molecular Biology vol. 66 (Humana Press, Totowa, NJ). Methods for mapping an epitope include but not limited to, X-ray crystallography and alanine scanning mutagenesis methods.

In certain embodiments, when such a competing antibody is present in excess, it blocks (e.g., reduces) the binding of a reference antibody to TGF-beta 1 by at least 10%, 15%, 20%, 25%, 30%, 35%, 40%, 45%, 50%, 55%, 60%, 65%, 70%, 75% or more. In some instances, binding is inhibited by at least 80%, 85%, 90%, 95%, or more. In certain embodiments, such a competing antibody binds to the same epitope (e.g., a linear epitope or a conformational epitope) that is bound by an anti-TGF-beta 1 antibody described herein. In further aspects, the reference antibody is TBA0946, TBA0947 or TBA1172.

In an exemplary competition assay, immobilized TGF-beta 1 is incubated in a solution comprising a first labeled antibody (a reference antibody) that binds to TGF-beta 1 (e.g., TBA0946, TBA0947 or TBA1172.) and a second unlabeled antibody that is being tested for its ability to compete with the first antibody for binding to TGF-beta 1. The second antibody may be present in a hybridoma supernatant. As a control, immobilized TGF-beta 1 is incubated in a solution comprising the first labeled antibody but not the second unlabeled antibody. After incubation under conditions permissive for binding of the first antibody to TGF-beta 1, excess unbound antibody is removed, and the amount of label associated with immobilized TGF-beta 1 is measured. If the amount of label associated with immobilized TGF-beta 1 is substantially reduced in the test sample relative to the control sample, then that indicates that the second antibody is competing with the first antibody for binding to TGF-beta 1. See Harlow and Lane (1988) Antibodies: A Laboratory Manual ch.14 (Cold Spring Harbor Laboratory, Cold Spring Harbor, NY).

In certain embodiments, Binding of an anti-TGF-beta 1 antibody to a cell surface latent TGF-beta 1 can be tested by known methods such as ELISA, Western blot, BIAcore, etc. For example, cells expressing latent TGF-beta 1 can be brought into contact with either anti-TGF-beta 1 antibodies directly conjugated with PE- or APC-, or unconjugated anti-TGF-beta 1 antibodies followed by PE- or APC-conjugated secondary antibodies, and the staining of sell surface latent TGF-beta 1 can be detected. See, e.g., Oida et al., PLoS One. 2010 Nov. 24; 5(11):e15523; Su et al, Hum Mol Genet. 2015 Jul. 15; 24(14):4024-36.

### 2. Activity Assays

In one aspect, assays are provided for identifying anti-TGF-beta 1 antibodies thereof having biological activity. Biological activity may include, e.g., inhibiting activation of TGF-beta 1, inhibiting the release of mature TGF-beta 1 from latent TGF-beta 1, inhibiting protease mediated release of mature TGF-beta 1 from latent TGF-beta 1, inhibiting protease mediated release of mature TGF-beta 1 from latent TGF-beta 1 without inhibiting protease mediated cleavage of the LAP region of latent TGF-beta 1, inhibiting protease mediated release of mature TGF-beta 1 from latent TGF-beta 1 without blocking access of a protease to latent TGF-beta 1, inhibiting protease mediated release of mature TGF-beta 1 from latent TGF-beta 1 while allowing a protease to cleave the LAP region of the latent TGF-beta 1, inhibiting protease mediated release of mature TGF-beta 1 from latent TGF-beta 1 without inhibiting or with partially inhibiting integrin mediated TGF-beta 1 activation, etc. Antibodies having such biological activity in vivo and/or in vitro are also provided.

In certain embodiments, an antibody of the invention is tested for such biological activity.

In some embodiments, whether a test antibody inhibits activation of latent TGF-beta 1, i.e., inhibits the release of mature TGF-beta 1 from latent TGF-beta 1, is determined by detecting mature TGF-beta 1 using a method known in the art such as electrophoresis, chromatography, immunoblot analysis, an enzyme-linked immunosorbent assay (ELISA), or mass spectrometry, after an activator of latent TGF-beta 1 (e.g., protease, integrin, other non-protease activator, etc.) is contacted with latent TGF-beta 1 in the presence or absence of the test antibody. It is also known that the activation latent TGF-beta 1, i.e., the release of mature TGF-beta 1 from latent TGF-beta 1, also occurs in the absence of an activator (spontaneous activation of latent TGF-beta 1). In some embodiments, whether a test antibody inhibits spontaneous activation of latent TGF-beta 1 is determined by detecting mature TGF-beta 1 using the method described above, after latent TGF-beta 1 is incubated with or without the test antibody. In some embodiments, where a decreased amount of mature TGF-beta 1 is detected in the presence of (or following contact with) the test antibody as compared to the amount detected in the absence of the test antibody, the test antibody is identified as an antibody that can inhibit the activation of latent TGF-beta 1. In an example, the amount of mature TGF-beta 1, either decreased or increased, can be measured in terms of concentration of mature TGF-beta 1 (for example, g/ml, mg/ml, microgram/ml, ng/ml, or pg/ml, etc.). In another example, the amount of mature TGF-beta, either decreased or increased, can be measured in terms of optical density (O.D.) (for example, at a wavelength in mm or nm, etc.) of a label directly or indirectly associated with mature TGF-beta.

In certain embodiments, inhibition of TGF-beta 1 activation includes at least a 5%, 10%, 15%, 20%, 25%, 30%, 35%, or 40% or greater decrease in the amount of mature TGF-beta 1 in the assay as compared to a negative control under similar conditions. In some embodiments, it refers to the inhibition of TGF-beta 1 activation i.e., the inhibition of the release of mature TGF-beta 1 of at least 45%, 50%, 55%, 60%, 65%, 70%, 75%, 80%, 85%, 90%, or 95% or greater.

In some embodiments, whether a test antibody inhibits activation of latent TGF-beta 1, i.e., inhibits the release of mature TGF-beta 1 from latent TGF-beta 1, is also determined by detecting mature TGF-beta 1 activity, for example, the activity of binding to TGF-beta 1 receptor, or the activity of mediating signal transduction in a cell expressing TGF-beta 1 receptor, etc. In some embodiments, binding of TGF-beta 1 to TGF-beta 1 receptor can be detected using a receptor binding assay. In some embodiments, the activity of mediating TGF-beta 1 signal transduction can be determined by detecting the activation of the TGF-beta 1/Smad pathway. Cells useful for such an assay can be those that express endogenous TGF-beta 1 receptor or that were generated by transfection of cells with a TGF-beta 1 receptor gene. For example, HEK-Blue™ TGF-beta cell which was used in the working examples described herein, or those that are genetically modified, transiently or stably, to express a transgene encoding TGF-beta 1 receptor can be used. TGF-beta 1 mediated signal transduction can be detected at any level in the signal transduction pathway, for example, by examining phosphorylation of Smad polypeptide, examining expression of a TGF-beta 1 regulated gene including a reporter gene, or measuring proliferation of a TGF-beta 1-dependent cell.

In some embodiments, the activity of mediating TGF-beta 1 signal transduction can also be determined by detecting the activation of the TGF-beta 1/Smad pathway, by examining phosphorylation of Smad polypeptide (see, e.g., Fukasawa et. al., Kidney International. 65(1):63-74 (2004), and Ganapathy et al., Molecular Cancer 26; 9:122 (2010)). In other embodiments, the activity of mediating TGF-beta 1 signal transduction can be determined by examining the ability of TGF-beta to inhibit cell migration in “wounded” monolayer cultures of BAE cells, examining the ability of TGF-beta to inihibit cell growth, examining the ability of TGF-beta to suppress plasminogen activator (PA) activity, examining the ability of TGF-beta to upregulate plasminogen activator inhibitor-1 (PAI-1), etc. (see Mazzieri et. al., Methods in Molecular Biology 142:13-27(2000))

Inhibition of TGF-beta 1 activation can also be detected and/or measured using the methods set forth and exemplified in the working examples. Using assays of these or other suitable types, test antibodies can be screened for those capable of inhibiting the activation of TGF-beta 1. In certain embodiments, inhibition of TGF-beta 1 activation includes at least a 5%, 10%, 15%, 20%, 25%, 30%, 35%, or 40% or greater decrease in TGF-beta 1 activation in the assay as compared to a negative control under similar conditions. In some embodiments, it refers to the inhibition of TGF-beta 1 activation of at least 45%, 50%, 55%, 60%, 65%, 70%, 75%, 80%, 85%, 90%, or 95% or greater. In certain embodiments, inhibition of TGF-beta 1 activation includes at least a 5%, 10%, 15%, 20%, 25%, 30%, 35%, or 40% or greater decrease in the amount of mature TGF-beta 1 detected in the assay as compared to a negative control under similar conditions. In some embodiments, it refers to the decrease in the amount of mature TGF-beta 1 of at least 45%, 50%, 55%, 60%, 65%, 70%, 75%, 80%, 85%, 90%, or 95% or greater.

In some embodiments, whether a test antibody inhibits the cleavage of the LAP portion of latent TGF-beta 1 is determined by detecting the cleavage product of latent TGF-beta 1 and/or non-cleaved latent TGF-beta 1 using various methods known in the art such as electrophoresis, chromatography, immunoblot analysis, an enzyme-linked immunosorbent assay (ELISA), or mass spectrometry, after a protease is contacted with latent TGF-beta 1 in the presence or absence of the test antibody. For example, where a protein tag (e.g., FLAG-tag, etc.) is added to N-terminal of the LAP region of latent TGF-beta 1, the portion to which the protein tag added is cut off when the protease mediated cleavage occurs. Therefore, the cleavage product of latent TGF-beta 1 can be detected by detecting latent TGF-beta 1 (or LAP region of latent TGF-beta 1) without the protein tag, and/or the non-cleaved latent TGF-beta 1 can be detected by detecting latent TGF-beta 1 with the protein tag.

For another example, where a protein tag (e.g., FLAG-tag, etc.) is added to the N-terminal of the LAP region of latent TGF-beta 1, and where the location of cleavage site by a protease is not near the N-terminal of the LAP region of latent TGF-beta 1, the LAP region with the protein tag becomes shortened when the protease mediated cleavage occurs. Therefore, the cleavage product of latent TGF-beta 1 can be detected by detecting latent TGF-beta 1 having a shortened LAP region (or shortened LAP region of latent TGF-beta 1) with the protein tag.

In some embodiments, where a decreased amount of the cleavage product of latent TGF-beta 1 is detected in the presence of (or following contact with) the test antibody as compared to the amount detected in the absence of the test antibody, the test antibody is identified as an antibody that can inhibit the cleavage of latent TGF-beta 1. Conversely, where the amount of the cleavage product of latent TGF-beta 1 is not significantly decreased in the presence of (or following contact with) the test antibody as compared to the amount detected in the absence of the test antibody, the test antibody is identified as an antibody that does not inhibit the cleavage of latent TGF-beta 1. In some embodiments, where an increased amount of the non-cleaved latent TGF-beta 1 is detected in the presence of (or following contact with) the test antibody as compared to the amount detected in the absence of the test antibody, the test antibody is identified as an antibody that can inhibit the cleavage of latent TGF-beta 1. Conversely, where an amount of non-cleaved latent TGF-beta 1 is not significantly increased in the presence of (or following contact with) the test antibody as compared to the amount detected in the absence of the test antibody, the test antibody is identified as an antibody that does not inhibit the cleavage of latent TGF-beta 1. In certain embodiments, whether a test antibody blocks access of a protease to latent TGF-beta 1 is determined by methods for the detection of protein interactions between the protease and latent TGF-beta 1, e.g., ELISAs or surface plasmon resonance (e.g. BIACORE(registered trademark)) or a similar technique (e.g. KinExa or OCTET(registered trademark)). Where a decreased interaction between the protease and latent TGF-beta 1 is detected in the presence of (or following contact with) the test antibody as compared to the interaction detected in the absence of the test antibody, the test antibody is identified as an antibody that can block access of the protease to latent TGF-beta 1.

In certain embodiments, non-inhibition of the cleavage of latent TGF-beta 1 includes at least a 5%, 10%, 15%, 20%, 25%, 30%, 35%, or 40% or greater increase in the amount of the cleavage product of latent TGF-beta 1 in the assay as compared to a negative control under similar conditions. In some embodiments, non-inhibition of the cleavage of latent TGF-beta 1 includes at least 50% or less, 45% or less, 40% or less, 35% or less, 30% or less, 25% or less, 20% or less, 15% or less, 10% or less, 5% or less increase in the amount of the non-cleaved latent TGF-beta 1 in the assay as compared to a negative control under similar conditions.

### 3. Methods for Screening

In one aspect, a method for screening an antibody of the invention comprises various assays described herein and known in the art. For example, a method for screening an anti-TGF-beta 1 antibody comprises:
(a) contacting a biological sample comprising latent TGF-beta 1 and a protease with a test antibody;
(b) detecting (i) whether a test antibody inhibits the cleavage of the LAP region of latent TGF-beta 1 and (ii) whether a test antibody inhibits activation of latent TGF-beta 1; and
(c) selecting the test antibody that inhibits activation of latent TGF-beta 1 without inhibiting protease mediated cleavage of the LAP portion of latent TGF-beta 1.

Alternatively, rather than steps (b) and (c) above, the method for screening an anti-TGF-beta 1 antibody comprises, e.g., steps (b) and (c) below:
(b) measuring (i) the amount of non-cleaved latent TGF-beta 1 and (ii) the amount of mature TGF-beta 1; and
(c) selecting the test antibody that inhibits a protease mediated release of mature TGF-beta 1 from latent TGF-beta 1 without inhibiting a protease mediated cleavage of the LAP region of latent TGF-beta 1, if the amount of non-cleaved latent TGF-beta 1 is not significantly increased and the amount of mature-TGF-beta 1 is decreased as compared to when the test antibody is absent.
Alternatively, rather than steps (b) and (c) above, the method for screening an anti-TGF-beta 1 antibody comprises, e.g., steps (b) and (c) below:
(b) measuring (i) the amount of cleavage product of latent TGF-beta 1 and (ii) the level of mature TGF-beta 1 activity; and
(c) selecting the test antibody that inhibits a protease mediated activation of latent TGF-beta 1 without inhibiting a protease mediated cleavage of the LAP region of latent TGF-beta 1, if the amount of cleavage product is not significantly decreased and the level of mature-TGF-beta 1 activity is decreased as compared to when the test antibody is absent.

Furthermore, the present invention provides a method for producing an anti-TGF-beta 1 antibody, which comprises, e.g., steps (d) and (e) below in addition to steps (a) to (c) above:
(d) obtaining amino acid sequence information of the anti-TGF-beta 1 antibody selected in step (c); and
(e) introducing a gene encoding the anti-TGF-beta 1 antibody into a host cell.

In this context, the term “not significantly increased/decreased”, e.g., in the phrases “the amount of non-cleaved latent TGF-beta 1 is not significantly increased” and “the amount of cleavage product (of latent TGF-beta 1) is not significantly decreased” means that the level/degree of the increase/decrease may be zero, or may not be zero but near zero, or may be very low enough to be able to be technically neglected or realistically/substantially considered to be zero by those skilled in the art. For example, in an immunoblotting analysis, when a researcher cannot detect or observe any significant signal/band (or a relatively high or strong signal) for non-cleaved latent TGF-beta 1, it is considered that the amount of non-cleaved latent TGF-beta 1 is “not significantly increased”, or the amount of cleavage product (of latent TGF-beta 1) is “not significantly decreased”. In addition, the term “not significantly increased/decreased” is interchangeably used with the term “not substantially increased/decreased”.

In some embodiments, whether a test antibody inhibits the cleavage of the LAP region of latent TGF-beta 1, and whether a test antibody inhibits activation of latent TGF-beta 1 can be determined by various assays described herein and known in the art.

### D. Immunoconjugates

The invention also provides immunoconjugates comprising an anti-TGF-beta 1 antibody herein conjugated to one or more cytotoxic agents, such as chemotherapeutic agents or drugs, growth inhibitory agents, toxins (e.g., protein toxins, enzymatically active toxins of bacterial, fungal, plant, or animal origin, or fragments thereof), or radioactive isotopes.

In one embodiment, an immunoconjugate is an antibody-drug conjugate (ADC) in which an antibody is conjugated to one or more drugs, including but not limited to a maytansinoid (see U.S. Pat. Nos. 5,208,020, 5,416,064 and European Patent EP 0 425 235 B1); an auristatin such as monomethylauristatin drug moieties DE and DF (MMAE and MMAF) (see U.S. Pat. Nos. 5,635,483 and 5,780,588, and 7,498,298); a dolastatin; a calicheamicin or derivative thereof (see U.S. Pat. Nos. 5,712,374, 5,714,586, 5,739,116, 5,767,285, 5,770,701, 5,770,710, 5,773,001, and 5,877,296; Hinman et al., Cancer Res. 53:3336-3342 (1993); and Lode et al., Cancer Res. 58:2925-2928 (1998)); an anthracycline such as daunomycin or doxorubicin (see Kratz et al., Current Med. Chem. 13:477-523 (2006); Jeffrey et al., Bioorganic & Med. Chem. Letters 16:358-362 (2006); Torgov et al., Bioconj. Chem. 16:717-721 (2005); Nagy et al., Proc. Natl. Acad. Sci. USA 97:829-834 (2000); Dubowchik et al., Bioorg. & Med. Chem. Letters 12:1529-1532 (2002); King et al., J. Med. Chem. 45:4336-4343 (2002); and U.S. Pat. No. 6,630,579); methotrexate; vindesine; a taxane such as docetaxel, paclitaxel, larotaxel, tesetaxel, and ortataxel; a trichothecene; and CC1065.

In another embodiment, an immunoconjugate comprises an antibody as described herein conjugated to an enzymatically active toxin or fragment thereof, including but not limited to diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolacca americana proteins (PAPI, PAPII, and PAP-S), Momordica charantia inhibitor, curcin, crotin, Saponaria officinalis inhibitor, gelonin, mitogellin, restrictocin, phenomycin, enomycin, and the tricothecenes.

In another embodiment, an immunoconjugate comprises an antibody as described herein conjugated to a radioactive atom to form a radioconjugate. A variety of radioactive isotopes are available for the production of radioconjugates. Examples include 211At, 1311, 125I, 90Y, 186Re, 188Re, 153Sm, 212Bi, 32P, 212Pb and radioactive isotopes of Lu. When the radioconjugate is used for detection, it may comprise a radioactive atom for scintigraphic studies, for example Tc-99m or 1231, or a spin label for nuclear magnetic resonance (NMR) imaging (also known as magnetic resonance imaging, MRI), such as iodine-123 again, iodine-131, indium-111, fluorine-19, carbon-13, nitrogen-15, oxygen-17, gadolinium, manganese or iron.

Conjugates of an antibody and cytotoxic agent may be made using a variety of bifunctional protein coupling agents such as N-succinimidyl-3-(2-pyridyldithio) propionate (SPDP), succinimidyl-4-(N-maleimidomethyl) cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al., Science 238:1098 (1987). Carbon-14-labeled 1-isothiocyanatobenzyl-3-methyldiethylene triaminepentaacetic acid (MX-DTPA) is an exemplary chelating agent for conjugation of radionuclide to the antibody. See WO94/11026. The linker may be a “cleavable linker” facilitating release of a cytotoxic drug in the cell. For example, an acid-labile linker, peptidase-sensitive linker, photolabile linker, dimethyl linker or disulfide-containing linker (Chari et al., Cancer Res. 52:127-131 (1992); U.S. Pat. No. 5,208,020) may be used.

The immunoconjugates or ADCs herein expressly contemplate, but are not limited to such conjugates prepared with cross-linker reagents including, but not limited to, BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, and sulfo-SMPB, and SVSB (succinimidyl-(4-vinylsulfone)benzoate) which are commercially available (e.g., from Pierce Biotechnology, Inc., Rockford, IL., U.S.A).

### E. Methods and Compositions for Diagnostics and Detection

In certain embodiments, any of the anti-TGF-beta 1 antibodies provided herein is useful for detecting the presence of TGF-beta 1, e.g., latent TGF-beta 1 in a biological sample. The term “detecting” as used herein encompasses quantitative or qualitative detection/measurement. In certain embodiments, a biological sample comprises a cell or tissue, such as serum, whole blood, plasma, biopsy sample, tissue sample, cell suspension, saliva, sputum, oral fluid, cerebrospinal fluid, amniotic fluid, ascites fluid, milk, colostrums, mammary gland secretion, lymph, urine, sweat, lacrimal fluid, gastric fluid, synovial fluid, peritoneal fluid, ocular lens fluid and mucus.

In one embodiment, an anti-TGF-beta 1 antibody for use in a method of diagnosis or detection is provided. In a further aspect, a method of detecting the presence of TGF-beta 1, e.g., latent TGF-beta 1 in a biological sample is provided. For example, the method of detecting the presence of latent TGF-beta 1 comprises:
(a) contacting a biological sample with an anti-TGF-beta 1 antibody of the present invention described herein under conditions permissive for binding of the anti-TGF-beta 1 antibody to latent TGF-beta 1; and
(b) detecting whether a complex is formed between the anti-TGF-beta 1 antibody and latent TGF-beta 1.

Such a method may be an in vitro or in vivo method. In one embodiment, an anti-TGF-beta 1 antibody is used to select subjects eligible for therapy with an anti-TGF-beta 1 antibody, e.g., where TGF-beta 1, e.g., latent TGF-beta 1 is a biomarker for selection of patients. That is, the anti-TGF-beta 1 antibody is useful as a diagnostic agent in targeting TGF-beta 1.

More specifically, the anti-TGF-beta 1 antibody is useful for the diagnosis of fibrosis, preferably myocardial fibrosis, pulmonary/lung fibrosis, liver fibrosis, renal fibrosis, skin fibrosis, ocular fibrosis and myelofibrosis. Anti-TGF-beta 1 antibodies of the invention are also useful for the diagnosis of cancer.

In some embodiments, the present invention provides a method of inhibiting release of mature TGF-beta 1 from latent TGF-beta 1 without inhibiting a cleavage of the LAP region of latent TGF-beta 1 mediated by protease in a biological sample, comprising contacting the biological sample containing latent TGF-beta 1 with the anti-TGF-beta 1 antibody of the present invention under conditions permissive for binding of the antibody to latent TGF-beta 1.

In certain embodiments, e.g., for the detection/diagnosis purposes, labeled anti-TGF-beta 1 antibodies are provided. Labels include, but are not limited to, labels or moieties that are detected directly (such as fluorescent, chromophoric, electron-dense, chemiluminescent, and radioactive labels), as well as moieties, such as enzymes or ligands, that are detected indirectly, e.g., through an enzymatic reaction or molecular interaction. Exemplary labels include, but are not limited to, the radioisotopes 32P, 14C, 125I, 3H, and 131I, fluorophores such as rare earth chelates or fluorescein and its derivatives, rhodamine and its derivatives, dansyl, umbelliferone, luceriferases, e.g., firefly luciferase and bacterial luciferase (U.S. Pat. No. 4,737,456), luciferin, 2,3-dihydrophthalazinediones, horseradish peroxidase (RP), alkaline phosphatase, beta-galactosidase, glucoamylase, lysozyme, saccharide oxidases, e.g., glucose oxidase, galactose oxidase, and glucose-6-phosphate dehydrogenase, heterocyclic oxidases such as uricase and xanthine oxidase, coupled with an enzyme that employs hydrogen peroxide to oxidize a dye precursor such as HRP, lactoperoxidase, or microperoxidase, biotin/avidin, spin labels, bacteriophage labels, stable free radicals, and the like.

### F. Pharmaceutical Formulations

Pharmaceutical formulations of an anti-TGF-beta 1 antibody as described herein are prepared by mixing such an antibody having the desired degree of purity with one or more optional pharmaceutically acceptable carriers (Remington's Pharmaceutical Sciences 16th edition, Osol, A. Ed. (1980)), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Exemplary pharmaceutically acceptable carriers herein further include interstitial drug dispersion agents such as soluble neutral-active hyaluronidase glycoproteins (sHASEGP), for example, human soluble PH-20 hyaluronidase glycoproteins, such as rHuPH20 (HYLENEX (registered trademark), Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

Exemplary lyophilized antibody formulations are described in U.S. Pat. No. 6,267,958. Aqueous antibody formulations include those described in U.S. Pat. No. 6,171,586 and WO2006/044908, the latter formulations including a histidine-acetate buffer.

The formulation herein may also contain more than one active ingredients as necessary for the particular indication being treated, preferably those with complementary activities that do not adversely affect each other. For example, it may be desirable to further provide immune check point inhibitors, e.g., an inhibitor of CTLA-4, PD-1, PD-L1, PD-L2, CD160, CD57, CD244, LAG-3, CD272, KLRG1, CD26, CD39, CD73, CD305, TIGIT, TIM-3, or VISTA. In some embodiments, immune check point inhibitors are, e.g., an anti-CTLA-4 antibody, an anti-PD-1 antibody, an anti-PD-L1 antibody, an anti-PD-L2 antibody, an anti-CD160 antibody, an anti-CD57 antibody, an anti-CD244 antibody, an anti-LAG-3 antibody, an anti-CD272 antibody, an anti-KLRG1 antibody, an anti-CD26 antibody, an anti-CD39 antibody, an anti-CD73 antibody, an anti-CD305 antibody, an anti-TIGIT antibody, an anti-TIM-3 antibody, or an anti-VISTA antibody. Preferably, the immune check point inhibitor is an anti-PD-1 antibody or an anti-PD-L1 antibody. In some embodiments, the anti-PD-1 antibody is Nivolumab, Pembrolizumab, or Cemiplimab. In some embodiments, the anti-PD-L1 antibody is Atezolizumab, Avelumab, or Durvalumab, preferably Atezolizumab. Such active ingredients are suitably present in combination in amounts that are effective for the purpose intended.

Active ingredients may be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semipermeable matrices of solid hydrophobic polymers containing the antibody, which matrices are in the form of shaped articles, e.g. films, or microcapsules.

The formulations to be used for in vivo administration are generally sterile. Sterility may be readily accomplished, e.g., by filtration through sterile filtration membranes.

### G. Therapeutic Methods and Compositions

Any of the anti-TGF-beta 1 antibodies provided herein may be used in therapeutic methods.

In one aspect, an anti-TGF-beta 1 antibody for use as a medicament is provided. In further aspects, an anti-TGF-beta 1 antibody for use in treating cancer or fibrosis (such as liver fibrosis, renal fibrosis, or lung fibrosis), etc., is provided. In certain embodiments, an anti-TGF-beta 1 antibody for use in a method of treatment is provided. In certain embodiments, the invention provides an anti-TGF-beta 1 antibody for use in a method of treating an individual having cancer or fibrosis (such as liver fibrosis, renal fibrosis, or lung fibrosis), etc., comprising administering to the individual an effective amount of the anti-TGF-beta 1 antibody. In one such embodiment, the method further comprises administering to the individual an effective amount of at least one additional therapeutic agent, e.g., as described below. In further embodiments, the invention provides an anti-TGF-beta 1 antibody for use in inhibiting protease mediated activation of latent TGF-beta 1. In certain embodiments, the invention provides an anti-TGF-beta 1 antibody for use in a method of inhibiting protease mediated activation of latent TGF-beta 1 in an individual comprising administering to the individual an effective of the anti-TGF-beta 1 antibody to inhibit protease mediated activation of latent TGF-beta 1. An “individual” according to any of the above embodiments is preferably a human.

In a further aspect, the invention provides for the use of an anti-TGF-beta 1 antibody in the manufacture or preparation of a medicament. In one embodiment, the medicament is for treatment of cancer or fibrosis (such as liver fibrosis, renal fibrosis, or lung fibrosis), etc. In a further embodiment, the medicament is for use in a method of treating cancer or fibrosis (such as liver fibrosis, renal fibrosis, or lung fibrosis), etc., comprising administering to an individual having cancer or fibrosis (such as liver fibrosis, renal fibrosis, or lung fibrosis), etc., an effective amount of the medicament. In one such embodiment, the method further comprises administering to the individual an effective amount of at least one additional therapeutic agent, e.g., as described below. In a further embodiment, the medicament is for inhibiting protease mediated activation of latent TGF-beta 1. In a further embodiment, the medicament is for use in a method of inhibiting protease mediated activation of latent TGF-beta 1 in an individual comprising administering to the individual an amount effective of the medicament to inhibit protease mediated activation of latent TGF-beta 1. An “individual” according to any of the above embodiments may be a human.

In a further aspect, the invention provides a method for treating a cancer or fibrosis (such as liver fibrosis, renal fibrosis, or lung fibrosis), etc. In one embodiment, the method comprises administering to an individual having such cancer or fibrosis (such as liver fibrosis, renal fibrosis, or lung fibrosis), etc., an effective amount of an anti-TGF-beta 1 antibody. In one such embodiment, the method further comprises administering to the individual an effective amount of at least one additional therapeutic agent, as described below. An “individual” according to any of the above embodiments may be a human.

In a further aspect, the invention provides a method for inhibiting protease mediated activation of latent TGF-beta 1 in an individual. In one embodiment, the method comprises administering to the individual an effective amount of an anti-TGF-beta 1 antibody to inhibit protease mediated activation of latent TGF-beta 1. In one embodiment, an “individual” is a human.

In a further aspect, the invention provides pharmaceutical formulations comprising any of the anti-TGF-beta 1 antibodies provided herein, e.g., for use in any of the above therapeutic methods. In one embodiment, a pharmaceutical formulation comprises any of the anti-TGF-beta 1 antibodies provided herein and a pharmaceutically acceptable carrier. In another embodiment, a pharmaceutical formulation comprises any of the anti-TGF-beta 1 antibodies provided herein and at least one additional therapeutic agent, e.g., as described below.

Antibodies of the invention can be used either alone or in combination with other agents in a therapy. For instance, an antibody of the invention may be co-administered with at least one additional therapeutic agent. In certain embodiments, an additional therapeutic agent is an immune check point inhibitors, e.g., an inhibitor of CTLA-4, PD-1, PD-L1, PD-L2, CD160, CD57, CD244, LAG-3, CD272, KLRG1, CD26, CD39, CD73, CD305, TIGIT, TIM-3, or VISTA. In some embodiments, immune check point inhibitors are, e.g., an anti-CTLA-4 antibody, an anti-PD-1 antibody, an anti-PD-L1 antibody, an anti-PD-L2 antibody, an anti-CD160 antibody, an anti-CD57 antibody, an anti-CD244 antibody, an anti-LAG-3 antibody, an anti-CD272 antibody, an anti-KLRG1 antibody, an anti-CD26 antibody, an anti-CD39 antibody, an anti-CD73 antibody, an anti-CD305 antibody, an anti-TIGIT antibody, an anti-TIM-3 antibody, or an anti-VISTA antibody. Preferably, the immune check point inhibitor is an anti-PD-1 antibody or an anti-PD-L1 antibody. In some embodiments, the anti-PD-1 antibody is Nivolumab, Pembrolizumab, or Cemiplimab. In some embodiments, the anti-PD-L1 antibody is Atezolizumab, Avelumab, or Durvalumab, preferably Atezolizumab. In some embodiments, a combination therapy comprising an anti-TGFbeta antibody of the invention and an immune check point inhibitor has additive or synergistic efficacy, e.g., additive or synergistic antitumor effect, compared to the anti-TGFbeta antibody monotherapy or the immune check point inhibitor monotherapy.

Such combination therapies noted above encompass combined administration (where two or more therapeutic agents are included in the same or separate formulations), and separate administration, in which case, administration of the antibody of the invention can occur prior to, simultaneously, and/or following, administration of the additional therapeutic agent or agents. In one embodiment, administration of the anti-TGF-beta 1 antibody and administration of an additional therapeutic agent occur within about one month, or within about one, two or three weeks, or within about one, two, three, four, five, or six days, of each other. Antibodies of the invention can also be used in combination with radiation therapy.

An antibody of the invention (and any additional therapeutic agent) can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g. by injections, such as intravenous or subcutaneous injections, depending in part on whether the administration is brief or chronic. Various dosing schedules including but not limited to single or multiple administrations over various time-points, bolus administration, and pulse infusion are contemplated herein.

Antibodies of the invention would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The antibody need not be, but is optionally formulated with one or more agents currently used to prevent or treat the disorder in question. The effective amount of such other agents depends on the amount of antibody present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

For the prevention or treatment of disease, the appropriate dosage of an antibody of the invention (when used alone or in combination with one or more other additional therapeutic agents) will depend on the type of disease to be treated, the type of antibody, the severity and course of the disease, whether the antibody is administered for preventive or therapeutic purposes, previous therapy, the patient's clinical history and response to the antibody, and the discretion of the attending physician. The antibody is suitably administered to the patient at one time or over a series of treatments. Depending on the type and severity of the disease, about 1 micro g/kg to 15 mg/kg (e.g. 0.1 mg/kg-10 mg/kg) of antibody can be an initial candidate dosage for administration to the patient, whether, for example, by one or more separate administrations, or by continuous infusion. One typical daily dosage might range from about 1 micro g/kg to 100 mg/kg or more, depending on the factors mentioned above. For repeated administrations over several days or longer, depending on the condition, the treatment would generally be sustained until a desired suppression of disease symptoms occurs. One exemplary dosage of the antibody would be in the range from about 0.05 mg/kg to about 10 mg/kg. Thus, one or more doses of about 0.5 mg/kg, 2.0 mg/kg, 4.0 mg/kg or 10 mg/kg (or any combination thereof) may be administered to the patient. Such doses may be administered intermittently, e.g. every week or every three weeks (e.g. such that the patient receives from about two to about twenty, or e.g. about six doses of the antibody). An initial higher loading dose, followed by one or more lower doses may be administered. However, other dosage regimens may be useful. The progress of this therapy is easily monitored by conventional techniques and assays.

### H. Articles of Manufacture

In another aspect of the invention, an article of manufacture containing materials useful for the treatment, prevention and/or diagnosis of the disorders described above is provided. The article of manufacture comprises a container and a label on or a package insert associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper pierceable by a hypodermic injection needle). At least one active ingredient in the composition is an antibody of the invention. The label or package insert indicates that the composition is used for treating the condition of choice. Moreover, the article of manufacture may comprise (a) a first container with a composition contained therein, wherein the composition comprises an antibody of the invention; and (b) a second container with a composition contained therein, wherein the composition comprises a further cytotoxic or otherwise therapeutic agent. The article of manufacture in this embodiment of the invention may further comprise a package insert indicating that the compositions can be used to treat a particular condition. Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

It is understood that any of the above articles of manufacture may include an immunoconjugate of the invention in place of or in addition to an anti-TGF-beta 1 antibody.

### EXAMPLES

### Example 1: Expression and Purification of Human or Mouse Latent TGF-Beta 1 and Mouse Latency Associated Peptide (LAP)

The sequences used for expression and purification are: human latent TGF-beta 1 (SEQ ID NO: 29, 30) and mouse latent TGF-beta 1 (SEQ ID NO: 31, 32), both of which have a signal sequence derived from rat serum albumin (SEQ ID NO: 33), C33S mutation, and a Flag-tag on the N-terminus of LAP.

Flag-tagged human latent TGF-beta 1 (hereinafter called “recombinant human latent TGF-beta 1”) or flag-tagged mouse latent TGF-beta 1 (hereinafter called “recombinant mouse latent TGF-beta 1”) was expressed transiently using FreeStyle293-F or Expi293F cell line (Thermo Fisher Scientific). Conditioned media expressing human or mouse latent TGF-beta 1 was applied to a column packed with anti-Flag M2 affinity resin (Sigma), and latent TGF-beta 1 was eluted with a Flag peptide (Sigma). Fractions containing human or mouse latent TGF-beta 1 were collected and subsequently subjected to a Superdex 200 gel filtration column (GE healthcare) equilibrated with 1×PBS. Fractions containing human or mouse recombinant latent TGF-beta 1 were then pooled and stored at −80 degrees C.

### Example 2: Identification of Human/Mouse Cross-Reactive Anti-Latent TGF-Beta 1 Antibody

Specific antibodies binding to both human and mouse latent TGF-beta 1 were prepared, selected, and assayed as follows: Twelve to sixteen week old NZW rabbits were first immunized intradermally with human and mouse recombinant latent TGF-beta 1 proteins (100 microgram/dose/rabbit). Two weeks after the initial immunization, four more weekly doses were given, alternating between mouse and human recombinant latent TGF-beta 1 proteins (50 microgram/dose/rabbit). One week after the final immunization, spleen and blood from immunized rabbits were collected. Recombinant human latent TGF-beta 1 protein was labelled in vitro with NHS-PEG4-Biotin (PIERCE, Cat No. 21329) for B cell sorting. Antigen-specific B cells were stained with a labelled antigen, sorted using FCM cell sorter (FACS aria III, BD), plated and cultured as described in WO2016098357. After 7-12 days of cultivation, B cell culture supernatants were collected for further analysis and cell pellets were cryopreserved.

Binder screening was performed using Octet RED96 System (Pall ForteBio). Batch 1 of 1408 B cell supernatants were screened for binding to recombinant mouse latent TGF-beta 1, and 149 lines showing binding to recombinant mouse latent TGF-beta 1 were selected for cloning (TBA0888-TBA1036). Batch 2 of 5338 B cell supernatants were screened for binding to human and mouse recombinant latent TGF-beta 1, 162 lines cell showing binding to either or both human and mouse recombinant latent TGF-beta 1 supernatants were selected for cloning (TBA1037-TBA1198).

For antibody gene cloning, RNA was purified from corresponding B cell pellets using ZR-96 Quick-RNA kit (ZYMO RESEARCH, Cat No. R1053). The DNA of their antibody heavy chain variable regions was amplified by reverse transcription PCR and recombined with F1332m heavy chain constant region (SEQ ID NO: 34). The DNA of their antibody light chain variable regions was amplified by reverse transcription PCR and recombined with hk0MC light chain constant region (SEQ ID NO: 35). Recombinant antibodies were expressed transiently in FreeStyle 293-F cells according to the manufacturer's instructions (Life technologies) and purified using AssayMAP Bravo platform with protein A cartridge (Agilent).

### Example 3: Antibody Screening for Anti-Latent TGF-Beta 1 Antibody Generation

ELISA detecting mature TGF-beta 1 (Human TGF-beta 1 Quantikine ELISA Kit, R&D systems) was used to assess plasmin mediated TGF-beta 1 activation. Mouse recombinant latent TGF-beta 1 was incubated with human plasmin (Calbiochem), with or without the presence of anti-latent TGF-beta 1 antibodies at 37 degrees C. for 1 hour. The content of mature TGF-beta 1 in the mixture was analyzed by ELISA described above. The detection was done according to the manufacturer's procedure. Anti-latent TGF-beta 1 antibodies that have inhibitory activity against plasmin mediated TGF-beta 1 activation were screened out (e.g., TBA0946, TBA0947, TBA1172, TBA1122, TBA1006, and TBA0898). The amino acid sequences of the H chain and L chain of TBA0946 are shown in SEQ ID NOs: 36 and 37, respectively, the amino acid sequences of the H chain and L chain of TBA0947 are shown in SEQ ID NOs: 38 and 39, respectively, and the amino acid sequences of the H chain and L chain of TBA1172 are shown in SEQ ID NOs: 40 and 41, respectively. Further, the amino acid sequences of the variable regions (VRs) and CDRs (HVRs) of TBA0946, TBA0947, and TBA1127 are shown below.

TABLE 2

| Name | VR | Amino acid sequence | SEQ ID NO |
|---|---|---|---|
| TBA0946 | VH | QSLEESGGRLVTPGTPLTLTCTTSGFSLN | 23 |
| | | NYYMNWVRQAPGKGLEWIGLTDNDGT | |
| | | TYYANWAKGRFTVSKTSTTVDLKMTSP | |
| | | TTEDTATYFCVRDAGYSRNLWGPGTLV | |
| | | TVSS | |
| TBA0946 | VL | AYDMTQTPASVEVAVGGTVTIKCQASQ | 24 |
| | | SISSSLAWYQQKPGQPPKQLIYRASTLAS | |
| | | GVSSRFKGSGSGTQFTLTISGVECADAA | |
| | | TYYCQQTYSWSGLDNVFGGGTKVEIK | |
| TBA0947 | VH | QSVEESGGRLVTPGTPLTLTCTVSGFSLS | 25 |
| | | SYAMNWVRQAPGKGLEWIGYIYTSGTT | |
| | | YHANWARGRFTISKTSTTVDLKMTSLTT | |
| | | EDTATYFCARGTGISDYYYWVMDLWGP | |
| | | GTLVTVSS | |
| TBA0947 | VL | AFELTQTPSSVEAAVGGTVTIKCQASQSI | 26 |
| | | STYLAWYQQRPGQPPKLLIYKASTLASG | |
| | | VPSRFKGSGSGTDFTLTISDLECADAATY | |
| | | FCQSYCDGDSVGFGGGTKVEIK | |
| TBA1172 | VH | QSVEESGGRLVTPGTPLTLTCTVSGFSLS | 27 |
| | | NYAMGWVRQAPGKGLEWIGIISSGGVS | |
| | | YYASWARGRFTISKTSTTVDLKITSPTPE | |
| | | DTGTYFCARGQVPSSDTYYVPFNLWGP | |
| | | GTLVTVSS | |
| TBA1172 | VL | DIAMTQTPASVEAAVGGTVTIKCQASQS | 28 |
| | | IGSDLAWYQQKPGQPPKLLIYRASTLAS | |
| | | GVPLRFKGSGSGTQFTLTISDLECADAAT | |
| | | YYCQTYYFTISSYGAFGGGTKVEIK | |

TABLE 3

| Name | HVR | Amino acid sequence | SEQ ID NO |
|---|---|---|---|
| TBA0946 | HVR H1 | NYYMN | 5 |
| TBA0946 | HVR H2 | LTDNDGTTYYANWAKG | 6 |
| TBA0946 | HVR H3 | DAGYSRNL | 7 |
| TBA0946 | HVR L1 | QASQSISSSLA | 8 |
| TBA0946 | HVR L2 | RASTLAS | 9 |
| TBA0946 | HVR L3 | QQTYSWSGLDNV | 10 |
| TBA0947 | HVR H1 | SYAMN | 11 |
| TBA0947 | HVR H2 | YIYTSGTTYHANWARG | 12 |
| TBA0947 | HVR H3 | GTGISDYYYWVMDL | 13 |
| TBA0947 | HVR L1 | QASQSISTYLA | 14 |
| TBA0947 | HVR L2 | KASTLAS | 15 |
| TBA0947 | HVR L3 | QSYCDGDSVG | 16 |
| TBA1172 | HVR H1 | NYAMG | 17 |
| TBA1172 | HVR H2 | IISSGGVSYYASWARG | 18 |
| TBA1172 | HVR H3 | GQVPSSDTYYVPFNL | 19 |
| TBA1172 | HVR L1 | QASQSIGSDLA | 20 |
| TBA1172 | HVR L2 | RASTLAS | 21 |
| TBA1172 | HVR L3 | QTYYFTISSYGA | 22 |

### Example 4: Expression and Purification of Recombinant Antibodies

Recombinant antibodies were expressed transiently using the FreeStyle293-F or Expi293F cell line (Thermo Fisher Scientific). Purification from the conditioned media expressing antibodies was done with a conventional method using protein A or protein G. Gel filtration was further conducted if necessary.

### Example 5: Characterization of Anti-Latent TGF-Beta 1 Antibody

### 1. Anti-Latent TGF-Beta 1 Antibody Bound to Cell Surface Latent TGF-Beta 1

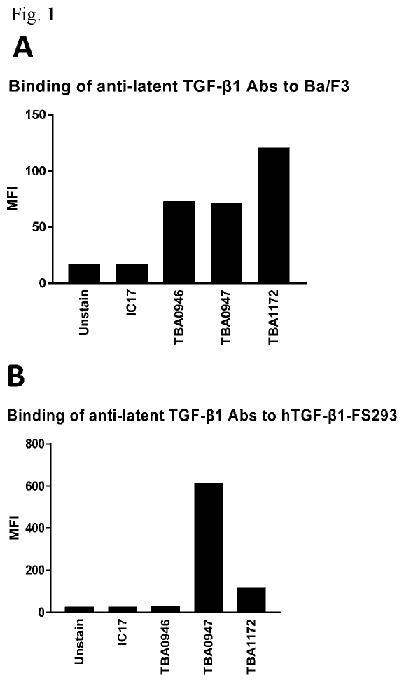

Binding of anti-latent TGF-beta 1 antibodies (TBA0946, TBA0947, or TBA1172) to cell surface latent TGF-beta 1 was tested by FACS using Ba/F3 cells or human TGF-beta 1 transfected FreeStyle™ 293-F cells (ThermoFisher). 10 microgram/mL anti-latent TGF-beta 1 antibodies were incubated with each cell line for 30 minutes at 4 degrees Celsius (C) and washed with FACS buffer (2% FBS, 2 mM EDTA in PBS). Anti-KLH antibody (IC17) was used for a negative control antibody. Goat F(ab′)2 anti-Human IgG, Mouse ads-PE (Southern Biotech, Cat. 2043-09) or Goat F(ab′)2 anti-Mouse IgG(H+L), Human Ads-PE (Southern Biotech, Cat. 1032-09) was then added and incubated for 30 minutes at 4 degrees C. and washed with FACS buffer. Data acquisition was performed on an FACS Verse (Becton Dickinson), followed by analysis using FlowJo software (Tree Star) and GraphPad Prism software (GraphPad). As shown in FIG. 1, TBA0946, TBA0947, and TBA1172 bound to mouse cell surface latent TGF-beta 1 expressed on Ba/F3 cells. TBA0947 and TBA1172 bound to human cell surface latent TGF-beta 1 expressed on FreeStyle™ 293-F cells.

### 2. Anti-Latent TGF-Beta 1 Antibody Inhibited Spontaneous Mouse Latent TGF-Beta 1 Activation

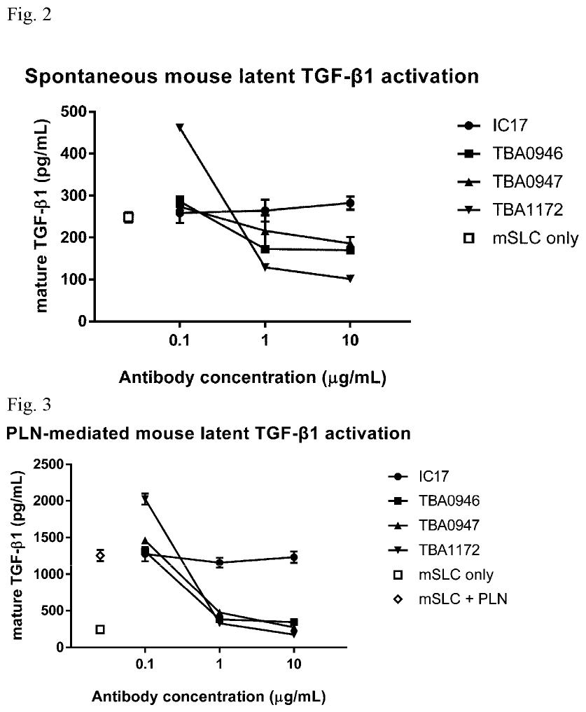

Recombinant mouse latent TGF-beta 1 was incubated with or without the presence of anti-latent TGF-beta 1 antibodies (TBA0946, TBA0947, or TBA1172) at 37 degrees C. for 1 hour. Spontaneous activation of mouse latent TGF-beta 1 was analyzed by mature TGF-beta 1 ELISA (Human TGF-beta 1 Quantikine ELISA Kit, R&D systems) according to manufacturer's procedure. As shown in FIG. 2, spontaneous activation of TGF-beta 1 was suppressed by anti-latent TGF-beta 1 antibodies.

### 3. Anti-Latent TGF-Beta 1 Antibody Inhibited Plasmin-Mediated Mouse Latent TGF Beta 1 Activation

Recombinant mouse latent TGF-beta 1 was incubated with plasmin (Calbiochem), with or without the presence of anti-latent TGF-beta 1 antibodies (TBA0946, TBA0947, or TBA1172) at 37 degrees C. for 1 hour. Plasmin-mediated mouse latent TGF-beta 1 activation and antibody-mediated inhibition was analyzed by mature TGF-beta 1 ELISA (Human TGF-beta 1 Quantikine ELISA Kit, R&D systems) according to manufacturer's procedure. As shown in FIG. 3, plasmin-mediated TGF-beta 1 activation was suppressed by anti-latent TGF-beta 1 antibodies.

### 4. Anti-Latent TGF-Beta 1 Antibody Inhibited Plasmin-Mediated Human Latent TGF-Beta 1 Activation

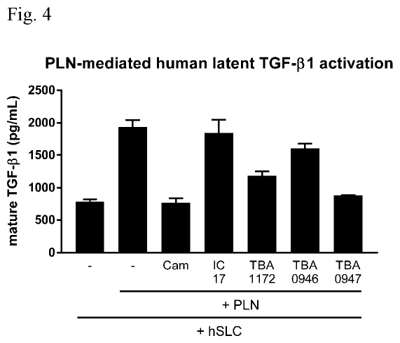

Recombinant human latent TGF-beta 1 was incubated with plasmin (Calbiochem), with or without the presence of anti-latent TGF-beta 1 antibodies (TBA0946, TBA0947, or TBA1172) at 37 degrees C. for 1 hour. Plasmin-mediated TGF-beta 1 activation and antibody mediated inhibition was analyzed by mature TGF-beta 1 ELISA (Human TGF-beta 1 Quantikine ELISA Kit, R&D systems) according to manufacturer's procedure. As shown in FIG. 4, plasmin-mediated human latent TGF-beta 1 activation was suppressed by anti-latent TGF-beta 1 antibodies.

### 5. Anti-Latent TGF-Beta 1 Antibody Inhibited MMP2 and MMP9-Mediated Mouse Latent TGF-Beta 1 Activation

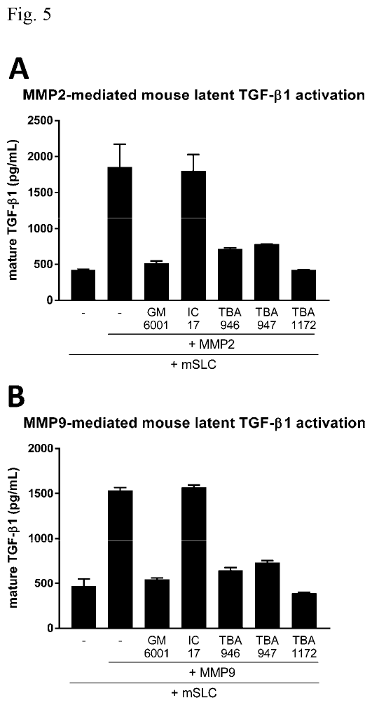

Recombinant mouse latent TGF-beta 1 was incubated with activated MMP2 or MMP9 (R&D systems), with or without the presence of anti-latent TGF-beta 1 antibodies at 37 degrees C. for 2 hours. MMP2 and MMP9-mediated mouse latent TGF-beta 1 activation and antibody mediated inhibition was analyzed by mature TGF-beta 1 ELISA (Human TGF-beta 1 Quantikine ELISA Kit, R&D systems) according to the manufacturer's procedure. Anti-KLH antibody (IC17) was used as negative control. GM6001 (TOCRIS) which is one of the MMP inhibitor was used as positive control. As shown in FIG. 5, MMP2 and MMP9-mediated mouse latent TGF-beta 1 activation was suppressed by the anti-latent TGF-beta 1 antibodies.

6. Anti-Latent TGF-Beta 1 Antibody Inhibited Mature TGF-Beta 1 Release without Preventing from Plasmin-Mediated Latent TGF-Beta 1 Propeptide Cleavage

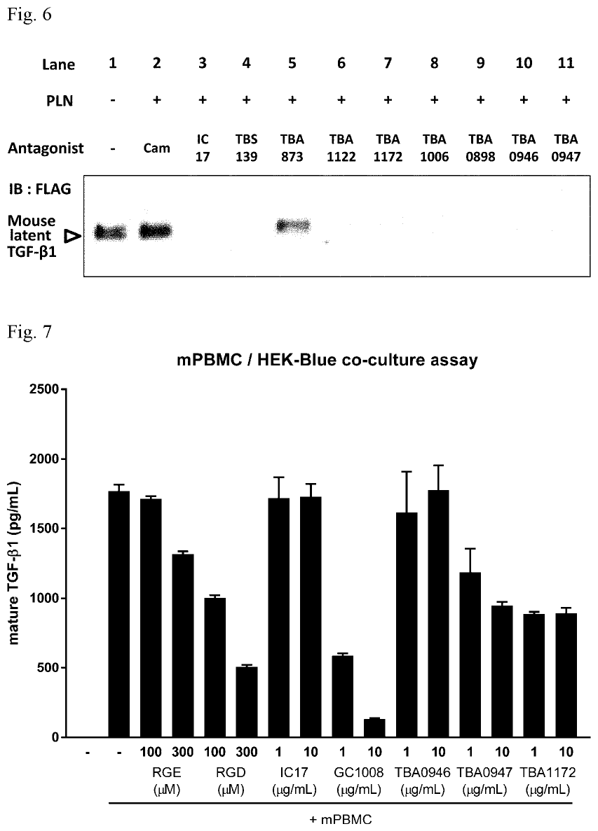

Recombinant mouse latent TGF-beta 1 was incubated with human plasmin (Calbiochem), with or without the presence of anti-latent TGF-beta 1 antibodies (TBA0946, TBA0947, or TBA1172) at 37 degrees C. for 1 hour. Anti-KLH antibody (IC17) was used for negative control. Camostat mesylate (TOCRIS) which is one of serine protease inhibitor, was used as positive control. Mixed with 4×SDS-PAGE sample buffer (Wako), the samples were heated at 95 degrees C. for 5 minutes and then loaded for SDS gel electrophoresis. Proteins were transferred to membrane by Trans-Blot® Turbo™ Transfer System (Bio-rad). Propeptide was detected using mouse anti-FLAG, M2-HRP antibody (Sigma-Aldrich). The membrane was incubated with an ECL substrate, and the image was taken by ImageQuant LAS 4000 (GE Healthcare). As shown in FIG. 6, propeptide cleavage by plasmin was not inhibited by TBA0946, TBA0947, and TBA1172.

### 7. Anti-Latent TGF-Beta 1 Antibody Partially Inhibited or Did not Inhibit Integrin-Mediated Latent TGF-Beta 1 Activation in Mouse PBMC

Mouse PBMC and HEK-Blue™ TGF-beta cell co-culture assay was conducted to detect integrin-mediated latent TGF-beta 1 activation. Mouse PBMC was isolated from mouse blood by using Histopaque-1083 density gradient medium (Sigma-Aldrich). HEK-Blue™ TGF-beta cells (Invivogen), which express Smad3/4-binding elements (SBE)-inducible SEAP reporter genes, allow the detection of bioactive TGF-beta 1 by monitoring the activation of Smad3/4. Active TGF-beta 1 stimulates the production of SEAP into cell supernatant. The quantity of SEAP secreted is assessed by using QUANTI-Blue™ reagent (Invivogen).

HEK-Blue™ TGF-beta cells were maintained in DMEM medium (Gibco) supplemented with 10% fetal bovine serum, 50 U/mL streptomycin, 50 microgram/mL penicillin, 100 microgram/mL Normocin, 30 microgram/mL of Blasticidin, 200 microgram/mL of HygroGold and 100 microgram/mL of Zeocin. During functional assay, the medium for cells was changed to assay medium (RPMI1640 with 10% FBS) and seeded to 96-well plate. Then the anti-latent TGF-beta 1 antibodies (TBA0946, TBA0947, or TBA1172) and mouse PBMC were applied to the wells and incubated together with HEK-Blue™ TGF-beta cells overnight. Then the cell supernatant was mixed with QUANTI-Blue™ and the optical density at 620 nm was measured in a colorimetric plate reader. The negative control antibody IC17 did not affect TGF-beta 1 activity whereas the anti-mature TGF-beta antibody GC1008 inhibited TGF-beta 1 activity. RGD peptide (GRRGDLATIH (SEQ ID NO: 100), GenScript) which is a decoy peptide to suppress integrin-mediated TGF-beta 1 activation strongly inhibited TGF-beta 1 activation in mouse PBMC. Furthermore, RGE control peptide (GRRGELATIH (SEQ ID NO: 101), GenScript) only suppressed the activation slightly. These results suggested that TGF-beta 1 activation in mouse PBMC largely depends on integrin mediated activation. As shown in FIG. 7, TBA0946 did not inhibit integrin-mediated TGF-beta 1 activation in mouse PBMC at all. However, TBA0947 and TBA1172 partially inhibited integrin-mediated TGF-beta 1 activation in mouse PBMC.

### 8. Biacore Analysis for Binding Activity Evaluation of Anti-Latent TGF-Beta 1 Antibodies

The binding activity of anti-latent TGF-beta 1 antibodies binding to human latent TGF-beta 1 at pH 7.4 was determined at 37 degrees C. using Biacore 8k instrument (GE Healthcare). Anti-human Fc (GE Healthcare) was immobilized onto all flow cells of a CM4 sensor chip using an amine coupling kit (GE Healthcare). Antibodies were captured onto the anti-Fc sensor surface, then recombinant human latent TGF-beta 1 was injected over the flow cell. All antibodies and analytes were prepared in ACES pH 7.4 containing 20 mM ACES, 150 mM NaCl, 1.2 mM CaCl₂), 0.05% Tween 20, 0.005% NaN3. The sensor surface was regenerated each cycle with 3M MgCl2. Binding activity was determined by processing and fitting the data to 1:1 binding model using Biacore 8K Evaluation software, version 1.1.1.7442 (GE Healthcare). The binding activities of anti-latent TGF-beta 1 antibodies binding to human latent TGF-beta 1 are shown in Table 4.

TABLE 4

| | Mouse latent TGFβ1 | | | Human latent TGFβ1 | | |
|---|---|---|---|---|---|---|
| Ab name | ka (M⁻¹s⁻¹) | kd (s⁻¹) | KD (M) | ka (M⁻¹s⁻¹) | kd (s⁻¹) | KD (M) |
| TBA0946bb-F1332m | 9.66E+05 | 7.66E−05 | 7.93E−11 | 2.75E+04 | 1.05E−02 | 3.80E−07 |
| TBA0947gg-F1332m | 2.56E+05 | 1.07E−05 | 4.19E−11 | 2.92E+05 | 2.24E−03 | 7.65E−09 |
| TBA1172aa-F1332m | 9.94E+05 | 9.13E−05 | 9.19E−11 | 2.61E+05 | 2.29E−02 | 8.80E−08 |

### 9. Anti-Latent TGF-Beta 1 Antibody Did not Bind to Mature TGF-Beta 1

Mouse mature TGF-beta 1 was purified from the purified recombinant mouse latent TGF-beta 1. The recombinant mouse latent TGF-beta 1 was acidified by addition of 0.1% trifluoroacetic acid (TFA) and applied to a Vydac 214TP C4 reverse phase column (Grace, Deerfield, IL, USA) and eluted with a TFA/CH3CN gradient. Fractions containing mature TGF-beta 1 were pooled, dried and stored at −80 degrees C. For reconstitution, mature TGF-beta 1 was dissolved in 4 mM HCl.

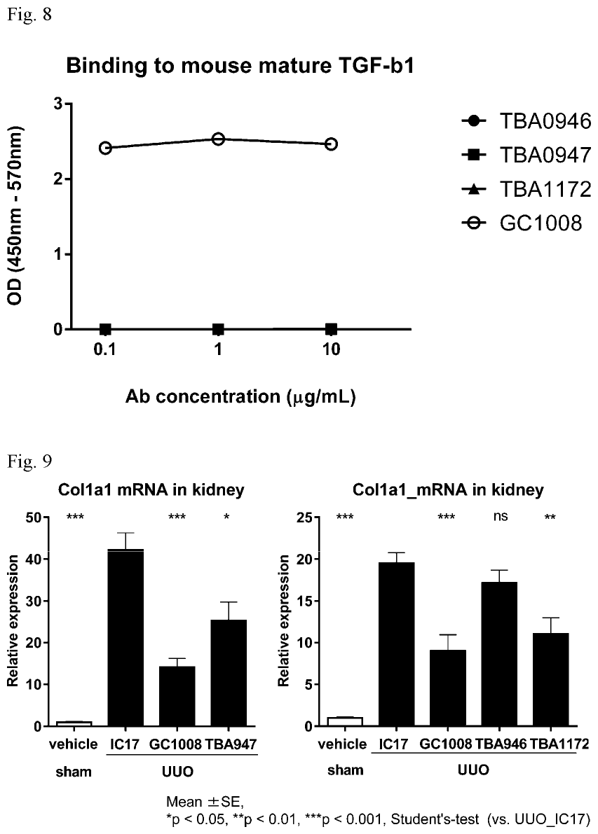

384-well plate was coated with mouse mature TGF-beta 1 for overnight at 4 degrees C. After four times washing with TBS-T, the plate was blocked with blocking buffer (1×TBS/tween20+0.5% BSA+1× Block ace) for 2 hours at room temperature. After four times washing with TBS-T, antibody solution was added into the plate and incubated for 2 hours at room temperature. After four times washing with TBS-T, a diluted secondary antibody (goat anti-human IgG-HRP Santa Cruz Cat.sc-2453) was add into the plate and incubated for 1 hour at room temperature. After four times washing with TBS-T, TMB solution was added into the plate and incubated for 15 min at room temperature, then 1N sulfuric acid was added to stop the reaction. The absorbance was measured at 450 nm/570 nm. As shown in FIG. 8, anti-latent TGF-beta 1 antibodies did not bind to mouse mature TGF-beta 1.

### Example 6: In Vivo Efficacy Assay

### 1. In Vivo Efficacy of Anti-Latent TGF-Beta 1 Antibodies in UUO Induced Mouse Renal Fibrosis Model

The in vivo efficacy of monoclonal antibodies TBA946, TBA947 and TBS 1172 were evaluated in Unilateral Ureteral Obstruction (UUO) mouse model which induces a progressive renal fibrosis.

Specific pathogen-free C57BL/6J male mice of 7 weeks of age were purchased from Japan Charles River Inc. (Kanagawa, Japan) and were acclimated for 1 week before the start of treatments. Animals were maintained at 20-26 degrees C. with a 12:12 h light/dark cycle and fed with a commercial standard diet (#CE-2; CLEA Japan Inc., Shizuoka, Japan) and tap water ad libitum.

UUO surgery was operated under isoflurane anesthetized condition. The left side of the abdomen was shaved and a vertical incision was made through the skin. A second incision was made through the peritoneum and that skin was also retracted to reveal the kidney. Using forceps, the kidney was brought to the surface and the left ureter was tied with surgical silk, twice, below the kidney. The ligated kidney was placed gently back into its correct anatomical position then peritoneum and skin were sutured. An analgesic agent was added to reduce animal affliction. In the sham operated group, peritoneum and skin were only incised and sutured.

All monoclonal antibodies were administered by intravenous injection once before the surgical operation. The sham operated group was administered vehicle. Antibodies were administered at 50 mg/kg. Anti-mature TGF-beta antibody GC1008 (as described in U.S. Pat. No. 8,383,780) was used as a positive control (50 mg/kg) and anti-KLH antibody IC17 was used as a negative control in this study (50 mg/kg). The animals were weighed and then killed by exsanguination under isoflurane anaesthesia on day 7. Blood samples were collected from the heart cavities or the postcaval vein and maintained at −80 degrees C. until assayed. The kidney was quickly removed and weighed. Part of the kidney tissue was snap-frozen in liquid nitrogen or on dry ice for molecular analyses.

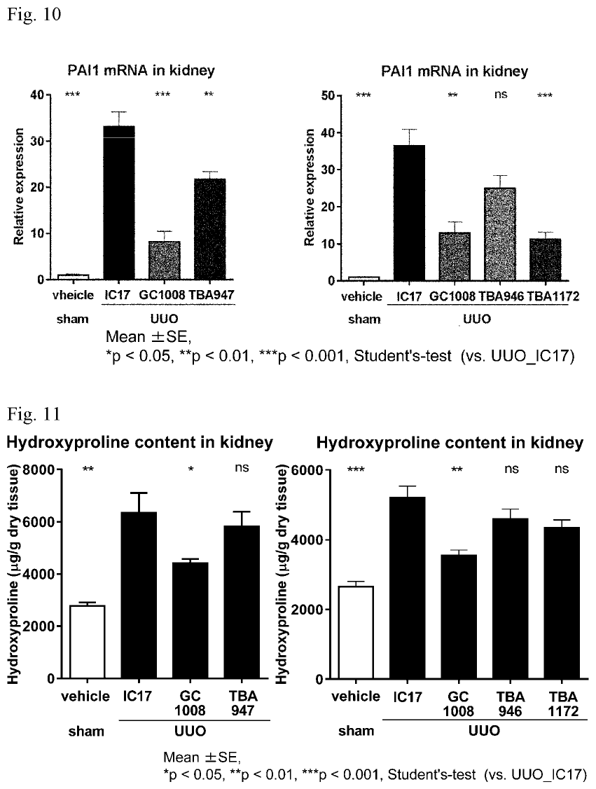

Total RNA was extracted from liver tissues using an RNeasy Mini Kit (Qiagen, Tokyo, Japan), and cDNA was synthesized using a Transcriptor Universal cDNA Master (Roche Applied Science, Tokyo, Japan). Gene expression was measured using the LightCycler 480 System (Roche Applied Science). Primers and Taq-Man probes for genes were purchased from Applied Biosystems. Mouse mitochondrial ribosomal protein L19 (MRPL19) was used as the endogenous reference for each sample. Relative mRNA expression values were calculated using double delta Ct analysis. The results of this experiment are shown in FIGS. 9 and 10. The antibodies inhibitory activity against kidney fibrosis were evaluated by collagen type 1 alpha 1 and plasminogen activator inhibitor 1 mRNA in kidney. UUO mice showed significant increases in collagen mRNA levels, and all antibodies (GC1008, TBS946, TBA947 and TBA1172) showed reduction.

The hydroxyproline contents in kidney, which is one of the amino acids included in collagen, was measured to evaluate the extramatrix deposition to the tissue. Wet kidney tissues were dried up at 110 degrees C. for 3 hours and weighed. Then, 6N HCl (100 microL/1 mg dry tissue) was added to the dried tissue and boiled at overnight. Samples were cleaned up by the filter and 10 microL of each samples were plated to the 96-well plate. The plate with samples was dried out at room temperature for overnight and hydroxyproline was measured using hydroxyproline assay kit (BioVision). The results of this experiment are shown in FIG. 11. Significant increase in the hydroxyproline content was observed in disease induced kidney, and all antibodies (GC1008, TBS946, TBA947 and TBA1172) inhibited kidney fibrosis.

Data are presented as mean+/−standard error of the mean (SEM). Statistical analysis was performed using analysis of variance (ANOVA) and Student's t-test. When P values were <0.05 or 0.01, differences were considered significant.

### 2. In Vivo Efficacy of Anti-Latent TGF-Beta 1 Antibodies in Bleomycin Induced Mouse Pulmonary Fibrosis Model

The in vivo efficacy of monoclonal antibody TBS1172 was evaluated in a Bleomycin (BLM) mouse model which induces a progressive pulmonary fibrosis.

Specific pathogen-free C57BL/6J male mice of 6 weeks of age were purchased from Japan Charles River Inc. (Kanagawa, Japan) and were acclimated for 1 week before the start of treatments. Animals were maintained at 20-26 degrees C. with a 12:12 h light/dark cycle and fed with a commercial standard diet (#CE-2; CLEA Japan Inc., Shizuoka, Japan) and tap water ad libitum.

Intratracheal instillation of BLM was conducted under isoflurane anesthetized condition. All monoclonal antibodies were administered by intravenous injection on 7 and 14 days after BLM instillation. Antibodies were administered at 50 mg/kg. Anti-mature TGF-beta antibody GC1008 (as described in U.S. Pat. No. 8,383,780) was used as a positive control (50 mg/kg) and anti-KLH antibody IC17 was used as a negative control in this study (50 mg/kg). The animals were weighed and then killed by exsanguination under isoflurane anaesthesia on day 21. Blood samples were collected from the heart cavities or the postcaval vein and maintained at −80 degrees C. until assayed. The lung was quickly removed and weighed. Part of the lung tissue was snap-frozen in liquid nitrogen or on dry ice for molecular analyses.

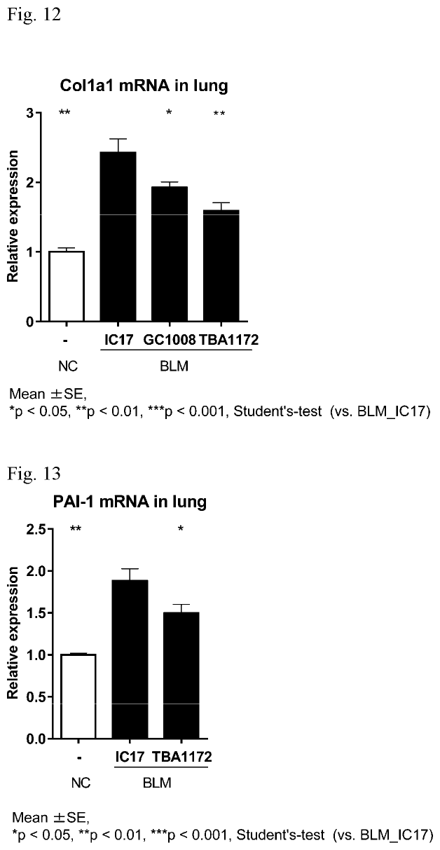

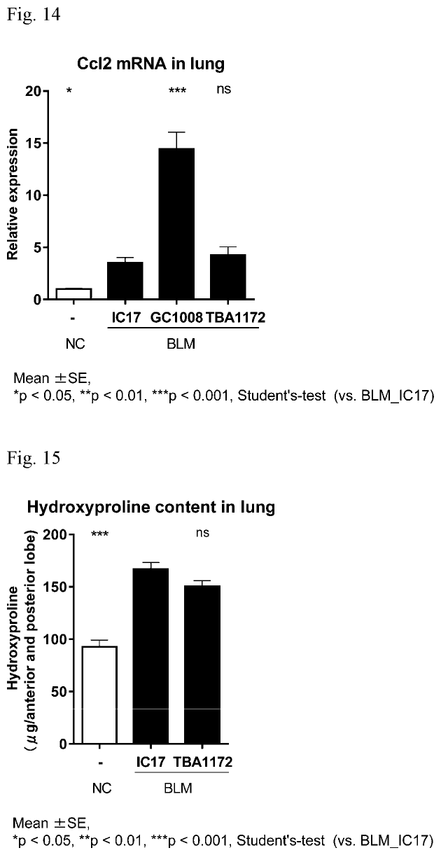

Total RNA was extracted from lung tissues, which was performed as with the previous method (detail in EXAMPLE 6-1). Mouse mitochondrial ribosomal protein L19 (MRPL19) was used as the endogenous reference for each sample. Relative mRNA expression values were calculated using double delta Ct analysis. The results of this experiment are shown in FIGS. 12, 13, and 14. The antibodies inhibitory activity against pulmonary fibrosis was evaluated by collagen type 1 alpha 1, plasminogen activator inhibitor 1, and chemokine ligand 2 mRNA in lung. BLM administered mice showed significant increases in collagen type 1 alpha 1 and plasminogen activator inhibitor 1 mRNA levels, and TBA1172 and GC1008 showed reduction. GC1008 dramatically enhanced chemokine ligand 2 mRNA in lung but TBA1172 did not.

The hydroxyproline contents in lung, which is one of the amino acids included in collagen, was measured to evaluate the extramatrix deposition to the tissue. Wet lung tissues were lyophilized and weighed. Then, 6N HCl (50 microL/1 mg dry tissue) was added to the dried tissue and boiled at 110 degrees C. for overnight. Samples were cleaned up by the filter, and each sample concentration of hydroxyproline was measured by mass spectrometry. The results of this experiment are shown in FIG. 15. Significant increase in the hydroxyproline content was observed in disease induced lung. TBA1172 tended to inhibit lung fibrosis.

Data are presented as mean+/−standard error of the mean (SEM). Statistical analysis was performed using analysis of variance (ANOVA) and Student's t-test. When P values were <0.05 or 0.01, differences were considered significant.

### Reference Example 1: Expression and Purification of Mouse Latency Associated Peptide (LAP)

Expression and purification of N-terminally Flag-tagged mouse LAP (SEQ ID NO: 42, 43) (hereinafter called “recombinant mouse latency associated protein (LAP)”) were performed exactly the same way as human or mouse recombinant latent TGF-beta 1, as described in EXAMPLE 1.

### Reference Example 2: Identification of Anti-Latent TGF-Beta 1 Antibody

Antibodies of the present invention were prepared, selected and assayed as follows:

Twelve to sixteen week old NZW rabbits were immunized intradermally with mouse recombinant latent TGF-beta 1 with an N-terminus FLAG tag or mouse recombinant TGF-beta 1 latency associated protein with an N-terminus FLAG tag (50-100 microgram/dose/rabbit). This dose was repeated 4 times over a period of 2 months. One week after the final immunization, the spleen and blood from the immunized rabbit was collected. Human recombinant latent TGF-beta 1 was labelled with NHS-PEG4-Biotin (PIERCE, Cat No. 21329) and antigen-specific B-cells were stained with the labelled antigen, sorted with FCM cell sorter (FACS aria III, BD), and plated in 96-well plates at a density of one cell/well together with 25,000 cells/well of EL4 cells (European Collection of Cell Cultures) and with rabbit T-cell conditioned medium diluted 20-fold, and were cultured for 7-12 days. EL4 cells were treated with mitomycin C (Sigma, Cat No. M4287) for 2 hours and washed 3 times in advance. The rabbit T-cell conditioned medium was prepared by culturing rabbit thymocytes in RPMI-1640 containing Phytohemagglutinin-M (Roche, Cat No. 1 1082132-001), phorbol 12-myristate 13-acetate (Sigma, Cat No. P1585) and 2% FBS. After cultivation, B-cell culture supernatants were collected for further analysis and pellets were cryopreserved.

ELISA assay was used to test the specificity of antibodies in B-cell culture supernatants. Human or mouse recombinant latent TGF-beta 1 was coated onto a 384-well MAXISorp (Nunc, Cat No. 164688) at 16 nM in PBS for 1 hour at room temperature. Plates were then blocked with Blocking One (Nacalai Tesque, Cat No. 03953-95) diluted 5-fold. The plates were washed with Tris-buffered Serine with 0.05% Tween-20 (TBS-T) and B-cell culture supernatants were added to the ELISA plates, incubated for 1 hr, and washed with TBS-T. Binding was detected by goat anti-rabbit IgG-Horseradish peroxidase (BETHYL, Cat No. A120-111P) followed by the addition of ABTS (KPL, Cat No. 50-66-06).

A total of 8,560 B-cell lines were screened for binding specificity to mouse and/or human latent TGF-beta 1 and 188 lines were selected and designated TBS001-188. RNA was purified from corresponding cell pellets by using ZR-96 Quick-RNA kits (ZYMO RESEARCH, Cat No. R1053). The DNAs of their antibody heavy chain variable regions were amplified by reverse transcription PCR and recombined with mF18 or F1332m heavy chain constant region (SEQ ID NO: 44, 36). The DNAs of their antibody light chain variable regions were amplified by reverse transcription PCR and recombined with mk1 or hk0MC light chain constant region (SEQ ID NO: 45, 37). Cloned antibodies were expressed in FreeStyle™ 293-F Cells (Invitrogen) and purified from culture supernatants to evaluate the functional activity.

### Reference Example 3: Antibody Screening for Anti-Latent TGF-Beta 1 Antibody Generation

ELISA detecting mature TGF-beta 1 (Human TGF-beta 1 Quantikine ELISA Kit, R&D systems) was used to assess prekallikrein and plasmin mediated TGF-beta 1 activation. Mouse recombinant latent TGF-beta 1 was incubated with human prekallikrein (Enzyme Research Laboratories) or plasmin (Calbiochem), with or without the presence of anti-latent TGF-beta 1 antibodies at 37 degrees C. for 1 hour. The content of mature TGF-beta 1 in the mixture was analyzed by ELISA described above. The detection was done according to the manufacturer's procedure. Anti-latent TGF-beta 1 antibodies that have inhibitory activity against prekallikrein and plasmin mediated TGF-beta 1 activation were screened out (e.g., TBS139, TBS182, TBA865 and TBA873). The amino acid sequences of the H chain and L chain of TBS139 are shown in SEQ ID NOs: 46 and 47, respectively, the amino acid sequences of the H chain and L chain of TBS182 are shown in SEQ ID NOs: 48 and 49, respectively, the amino acid sequences of the H chain and L chain of TBA865 are shown in SEQ ID NOs: 50 and 51, respectively, and the amino acid sequences of the H chain and L chain of TBA873 are shown in SEQ ID NOs: 52 and 53, respectively. Further, the amino acid sequences of the variable regions (VRs) and CDRs (HVRs) of TBS139 and TBS182 are shown below.

TABLE 5

| | | | SEQ |
|---|---|---|---|
| | | | ID |
| Name | VR | Amino acid sequence | NO |
| TBS139 | VH | QSLEESGGDLVKPGASLTLTCTASGFSFSS | 54 |
| | | YWMSWVRQAPGKGLEYIGMISIIGTTYYA | |
| | | SWAKGRFTISKTSSTTVTLQVTSLTAADTA | |
| | | TYFCARGYVSNSNYWGLWGPGTLVTVSS | |
| TBS139 | VL | AYDMTQTPASVEVAVGGTVTIKCQASQNI | 55 |
| | | GSNLAWYQQKPGQPPKLLIYYASDLASGV | |
| | | PSRFKGSGSGTRYTLTISGVQCDDAATYY | |
| | | CQQYWSSSNVDNTFGGGTEVVVK | |
| TBS182 | VH | QSLEESGGDLVKPGASLTLTCTASGFSFSR | 56 |
| | | SYYMCWVRQAPGKGLEWIACIYGGSSGS | |
| | | TWYASWAKGRFTISKTSSTTVTLQMTSLT | |
| | | AADTATYFCARDIGNYYRWWGPGTLVTV | |
| | | QS | |
| TBS182 | VL | AVVLTQTTSPVSAPVGGTVTIKCQASKNIY | 57 |
| | | SLLAWYQQKPGQPPKLLIYDASTLASGVP | |
| | | SRFKGSGSGTQFTLTISDLECADAATYYCQ | |
| | | SYYDSSSSADLSTFGGGTEVVVK | |

TABLE 6

| Name | HVR | Amino acid sequence | SEQ ID NO |
|---|---|---|---|
| TBS139 | HVR H1 | SYWMS | 58 |
| TBS139 | HVR H2 | MISIIGTTYYASWAKG | 59 |
| TBS139 | HVR H3 | GYVSNSNYWGL | 60 |
| TBS139 | HVR L1 | QASQNIGSNLA | 61 |
| TBS139 | HVR L2 | YASDLAS | 62 |
| TBS139 | HVR L3 | QQYWSSSNVDNT | 63 |
| TBS182 | HVR H1 | RSYYMC | 64 |
| TBS182 | HVR H2 | CIYGGSSGSTWYASWAKG | 65 |
| TBS182 | HVR H3 | DIGNYYRW | 66 |
| TBS182 | HVR L1 | QASKNIYSLLA | 67 |
| TBS182 | HVR L2 | DASTLAS | 68 |
| TBS182 | HVR L3 | QSYYDSSSSADLST | 69 |

### Reference Example 4: Expression and Purification of Recombinant Antibodies

Recombinant antibodies were expressed transiently using FreeStyle293-F or Expi293F cell line (Thermo Fisher Scientific). Purification from the conditioned media expressing antibodies was done with a conventional method using protein A or protein G. Gel filtration was further conducted if necessary.

### Reference Example 5: Characterization of Anti-Latent TGF-Beta 1 Antibody

### 1. Anti-Latent TGF-Beta 1 Antibody Inhibited Prekallikrein and Plasmin Mediated TGF-Beta 1 Activation

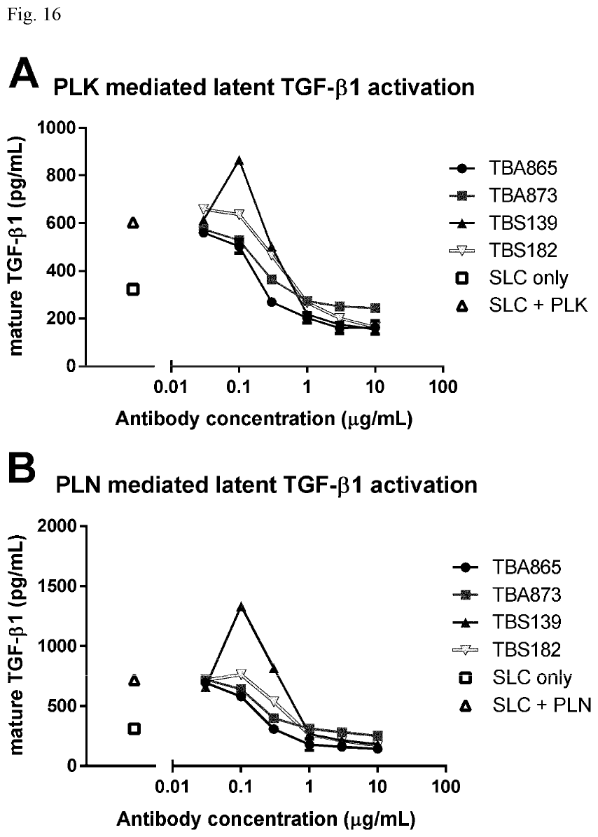

Mouse recombinant latent TGF-beta 1 was incubated with human prekallikrein (Enzyme Research Laboratories) or plasmin (Calbiochem), with or without the presence of anti-latent TGF-beta 1 antibodies (TBS139, TBS182, TBA865 or TBA873) at 37 degrees C. for 1 hour. Prekallikrein and plasmin mediated TGF-beta 1 activation and antibody mediated inhibition was analyzed by mature TGF-beta 1 ELISA (Human TGF-beta 1 Quantikine ELISA Kit, R&D systems) according to the manufacturer's procedure. As shown in FIG. 16, prekallikrein and plasmin mediated TGF-beta 1 activation was suppressed by the anti-latent TGF-beta 1 antibodies (TBS139, TBS182, TBA865 and TBA873).

### 2. Anti-Latent TGF-Beta 1 Antibody Inhibited Spontaneous TGF-Beta 1 Activation

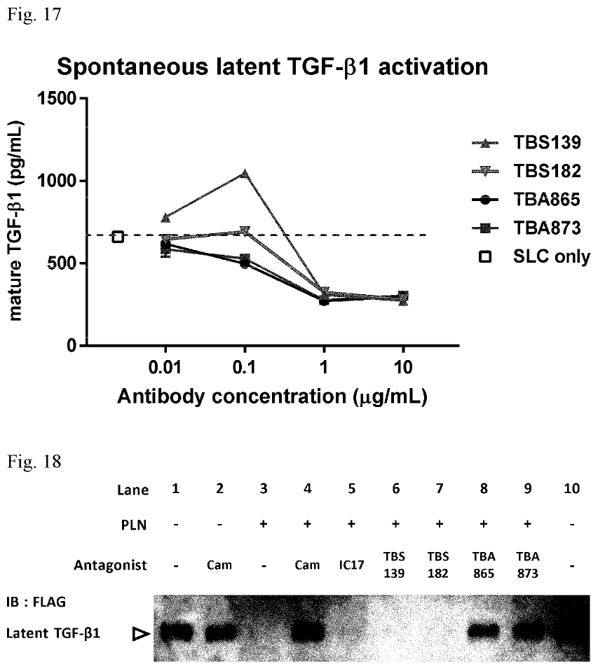

Mouse recombinant latent TGF-beta 1 was incubated with or without the anti-latent TGF-beta 1 antibodies (TBS139, TBS182, TBA865 or TBA873) at 37 degrees C. for 1 hour. Spontaneous activation of TGF-beta 1 was analyzed by mature TGF-beta 1 ELISA (Human TGF-beta 1 Quantikine ELISA Kit, R&D systems) according to the manufacturer's procedure. As shown in FIG. 17, Spontaneous activation of TGF-beta 1 was suppressed by the anti-latent TGF-beta 1 antibodies (TBS139, TBS182, TBA865 and TBA873).

### 3. Anti-Latent TGF-Beta 1 Antibody Inhibited Prekallikrein and Plasmin Mediated TGF-Beta 1 Propeptide Cleavage

Mouse recombinant latent TGF-beta 1 was incubated with human plasmin (Calbiochem), with or without the presence of anti-latent TGF-beta 1 antibodies (TBS139, TBS182, TBA865 or TBA873) at 37 degrees C. for 1 hour. Anti-KLH antibody (IC17) was used for a negative control. Camstat mesylate (TOCRIS) which is one of serine protease inhibitors was used for a positive control. Mixed with 4×SDS-PAGE sample buffer (Wako), the samples were heated at 95 degrees C. for 5 minutes and then loaded for SDS gel electrophoresis. Proteins were transferred to a membrane by Trans-Blot(registered trademark) Turbo™ Transfer System (Bio-rad). The propeptide was detected using M2 anti-FLAG antibody (Sigma-Aldrich), which was then detected by anti-mouse IgG-HRP (Santa Cruz). The membrane was incubated with an ECL substrate, and the image was taken by ImageQuant LAS 4000 (GE Healthcare). As shown in FIG. 18, the non-cleaved latent TGF-beta 1 (i.e., LAP region with FLAG-tag) was detected with the presence of TBA865 and TBA873 but not with the presence of TBS139 and TBS182, showing that propeptide cleavage by plasmin was only inhibited by TBA865 and TBA873 but not by TBS139 and TBS182.

### 4. Anti-Latent TGF-Beta 1 Antibody Partially Inhibited or Did not Inhibit Integrin Mediated TGF-Beta 1 Activation in Mouse PBMC

Mouse PBMC and HEK-Blue™ TGF-beta cell co-culture assay was done to detect integrin mediated latent TGF-beta 1 activation. Mouse PBMC was isolated from mouse blood by using Histopaque-1083 density gradient medium (Sigma-Aldrich). HEK-Blue™ TGF-beta cells (Invivogen), which express Smad3/4-binding elements (SBE)-inducible SEAP reporter genes, allow the detection of bioactive TGF-beta 1 by monitoring the activation of Smad3/4. Active TGF-beta 1 stimulates the production of SEAP into cell supernatants. The quantity of SEAP secreted is assessed by using QUANTI-Blue™ reagent (Invivogen).

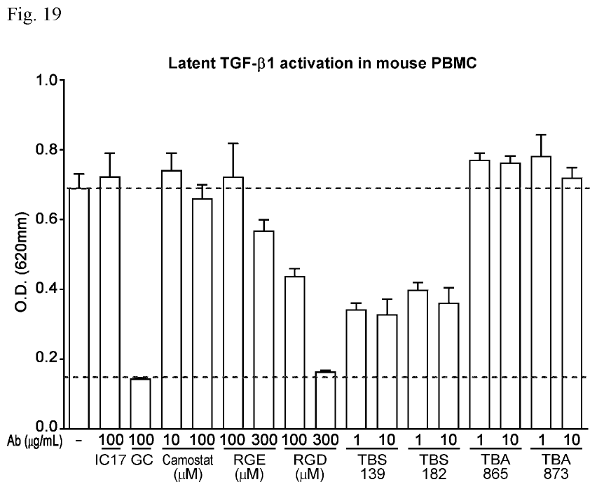

HEK-Blue™ TGF-beta cells were maintained in DMEM medium (Gibco) supplemented with 10% fetal bovine serum, 50 U/mL streptomycin, 50 micro g/mL penicillin, 100 micro g/mL Normocin™, 30 micro g/mL of Blasticidin, 200 micro g/mL of HygroGold™ and 100 micro g/mL of Zeocin™. During functional assay, the medium for cells was changed to assay medium (RPMI1640 with 10% FBS) and seeded to 96-well plates. Then, the anti-latent TGF-beta 1 antibodies (TBS139, TBS182, TBA865 or TBA873) and mouse PBMC were applied to the wells and incubated together with HEK-Blue™ TGF-beta cells overnight. Then, the cell supernatant was mixed with QUANTI-Blue™ and the optical density at 620 nm was measured in a colorimetric plate reader. It was proven that TGF-beta 1 activation in mouse PBMC largely depends on integrin mediated activation. As shown in FIG. 19, the negative control antibody IC17 did not affect TGF-beta 1 activity whereas the anti-mature TGF-beta 1 antibody GC1008 (shown as “GC” in FIG. 19) inhibited TGF-beta 1 activity. Camostat mesylate protease inhibitor did not suppress TGF-beta 1 activation at all. On the other hand, RGD peptide (GRRGDLATIH (SEQ ID NO: 100), GenScript) which is a decoy peptide to suppress integrin mediated TGF-beta 1 activation strongly inhibited TGF-beta 1 activation in mouse PBMC. Furthermore, RGE control peptide (GRRGELATIH (SEQ ID NO: 101), GenScript) only suppressed activation slightly. These results suggested that TGF-beta 1 activation in mouse PBMC largely depends on integrin mediated activation.

As shown in FIG. 19, TBA865 and TBA873 did not inhibit integrin mediated TGF-beta 1 activation in mouse PBMC at all. However TBS139 and TBS182 partially inhibited integrin mediated TGF-beta 1 activation in mouse PBMC.

### 5. Biacore Analysis for Binding Affinity Evaluation of Anti-Latent TGF-Beta 1 Antibodies

The affinities of anti-latent TGF-beta 1 antibodies binding to human or mouse latent TGF-beta 1 at pH 7.4 were determined at 37 degrees C. using Biacore T200 instrument (GE Healthcare). Anti-human Fc (GE Healthcare) was immobilized onto all flow cells of a CM4 sensor chip using an amine coupling kit (GE Healthcare). All antibodies and analytes were prepared in ACES pH 7.4 containing 20 mM ACES, 150 mM NaCl, 1.2 mM CaCl₂), 0.05% Tween 20, 0.005% NaN3. Each antibody was captured onto the sensor surface by anti-human Fc. Antibody capture levels were aimed at 300 resonance unit (RU). For TBA865 and TBA873, recombinant human or mouse latent TGF-beta 1 was injected at 12.5 to 200 nM prepared by two-fold serial dilution, followed by dissociation. For TBS139 and TBS182, recombinant human or mouse latent TGF-beta 1 was injected at 3.125 to 50 nM prepared by two-fold serial dilution, followed by dissociation. The sensor surface was regenerated each cycle with 3M MgCl2. Binding affinities were determined by processing and fitting the data to 1:1 binding model using Biacore T200 Evaluation software, version 2.0 (GE Healthcare).

The affinities of anti-latent TGF-beta 1 antibodies binding to human or mouse latent TGF-beta 1 are shown in TABLE 7.

TABLE 7

| | Mouse latent TGF-β1 | | | Human latent TGF-β1 | | |
|---|---|---|---|---|---|---|
| Ab name | ka (M⁻¹s⁻¹) | kd (s⁻¹) | KD (M) | ka (M⁻¹s⁻¹) | kd (s⁻¹) | KD (M) |
| TBS139 | 1.76E+06 | 3.96E−05 | 2.25E−11 | n.b.* | | |
| TBS182 | 1.10E+06 | 5.32E−05 | 4.82E−11 | n.b.* | | |
| TBA865 | 3.41E+05 | 5.37E−05 | 1.58E−10 | 4.10E+05 | 6.55E−05 | 1.60E−10 |
| TBA873 | 1.87E+05 | 5.15E−05 | 2.76E−10 | 2.30E+05 | 6.72E−05 | 2.92E−10 |

*n.b. no binding

### 6. Biacore in Tandem Blocking Assay

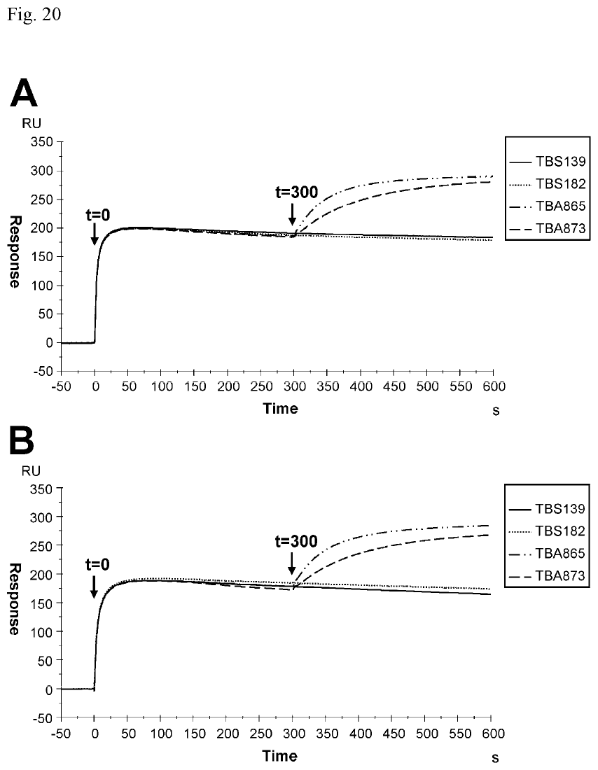

Biacore in-tandem blocking assay was performed to characterize the binding epitope of TBA865, TBA873, TBS139, and TBS182. The assay was performed on Biacore T200 instrument (GE Healthcare) at 25 degrees C. in ACES pH 7.4 buffer containing 20 mM ACES, 150 mM NaCl, 1.2 mM CaCl₂), 0.05% Tween 20, 0.005% NaN3. Monoclonal ANTI-FLAG(registered trademark) M2 antibody (Sigma) was immobilized onto Flow cell 1 and 2 of a CM5 sensor chip using an amine coupling kit (GE Healthcare). Mouse recombinant latent TGF-beta 1 with an N-terminal FLAG-tag was captured around 200 response unit (RU) at Flow cell 2. Flow cell 1 was used as a reference flow cell. Then, 500 nM of TBS139 or TBS182 at a saturating concentration was injected for 5 min and followed by an identical injection of 500 nM TBA865 or TBA873 as a competing mAb. An identical injection of TBS139 or TBS182 mAb was used as a reference of self-blocking. The sensor surface was regenerated each cycle with 100 mM Gly-HCl pH 2.4, 0.5 M NaCl. A binding response greater than that observed for the identical injection of TBS139 or TBS182 was indicative of binding to different epitopes whereas a response lower than or similar to those observed for the identical injection was indicative of binding to the same or overlapping or adjacent epitopes. Results of this assay are shown in FIG. 20.

### Reference Example 6: In Vivo Efficacy Assay

### 1. In Vivo Efficacy of Anti-Latent TGF-Beta 1 Antibodies in a CDAHFD-Induced NASH/Liver Fibrosis Mouse Model

The in vivo efficacy of monoclonal antibodies TBS139 and TBS182 was evaluated in a NASH/liver fibrosis model. All experimental animal care and handling were performed in accordance with the recommendations in the Guidelines for the Care and Use of Laboratory Animals at Chugai Pharmaceutical Co. Ltd., which is accredited by the Association for Assessment and Accreditation of Laboratory Animal Care International.

Specific-pathogen-free C57BL/6J male mice of 5 weeks of age were purchased from Japan SLC Inc. (Shizuoka, Japan) and were acclimated for 1 week before the start of treatment. Animals were housed at 20-26 degrees C. with a 12:12 h light/dark cycle and fed with a commercial standard diet (#CE-2; CLEA Japan Inc., Shizuoka, Japan) and tap water ad libitum. The test diet, a choline-deficient, L-amino acid-defined, high-fat diet (CDAHFD; #A06071302), was purchased from Research Diets (New Brunswick, NJ, USA). During the study, 10 groups were fed CDAHFD (n=8), and 1 group was fed CE-2 as a normal control group.

The monoclonal antibodies were given at various doses (2, 10, 50 mg/kg) by intravenous injection once per week for two weeks. Anti-mature TGF-beta antibody GC1008 (as described in U.S. Pat. No. 8,383,780) was used at 10 mg/kg as a positive control and anti-KLH antibody IC17 was used at 100 mg/kg as a negative control in this study. The animals were weighed and then killed by exsanguination under isoflurane anaesthesia on Day 14. Blood samples were collected from the heart cavity or the postcaval vein and maintained at −80 degrees C. until assayed. The liver was quickly removed and weighed. Part of the liver tissue was snap-frozen in liquid nitrogen or on dry ice for molecular analyses.

Total RNA was extracted from liver tissues using an RNeasy Mini Kit (Qiagen, Tokyo, Japan), and cDNA was synthesized using a Transcriptor Universal cDNA Master (Roche Applied Science, Tokyo, Japan). Gene expression was measured using the LightCycler 480 System (Roche Applied Science). Primers and Taq-Man probes for genes were purchased from Applied Biosystems. Mouse glyceraldehyde 3-phosphate dehydrogenase (GAPDH) was used as the endogenous reference for each sample. Relative mRNA expression values were calculated using double delta Ct analysis.

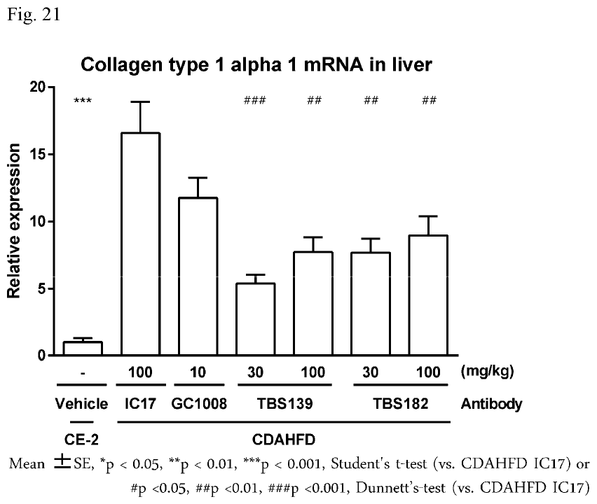

The results of this experiment are shown in FIG. 21. For the antibodies, the inhibitory activity against liver fibrosis was evaluated from the expression level of collagen type 1 alpha 1 mRNA in liver. CDAHFD significantly increased collagen mRNA level in the mice, and all three antibodies (GC1008, TBS139, and TBS182) reduced the level.

Data are presented as mean+/−standard error of the mean (SEM). Statistical analysis was performed using analysis of variance (ANOVA) and Student's t-test. P values of <0.05, <0.01, or <0.001 were considered significant.

### 2. In Vivo Efficacy of Anti-Latent TGF-Beta 1 Antibodies in a UUO-Induced Renal Fibrosis Mouse Model

The in vivo efficacy of monoclonal antibodies TBS139 and TBS182 was evaluated in a mouse model in which Unilateral Ureteral Obstruction (UUO) had induced progressive renal fibrosis.

Specific-pathogen-free C57BL/6J male mice of 7 weeks of age were purchased from Japan Charles River Inc. (Kanagawa, Japan) and were acclimated for 1 week before the start of treatment. Animals were housed at 20-26 degrees C. with a 12:12 h light/dark cycle and fed with a commercial standard diet (#CE-2; CLEA Japan Inc., Shizuoka, Japan) and tap water ad libitum.

UUO surgery was performed under an isoflurane anesthetized condition. The left side of the abdomen was shaved and a vertical incision was made through the skin. A second incision was made through the peritoneum, which was then retracted to reveal the kidney. The kidney was brought to the surface with forceps, and the left ureter was tied with surgical silk, twice, below the kidney. The ligated kidney was placed gently back into its correct anatomical position then peritoneum and skin were sutured. Analgesic agent was applied to reduce animal affliction. In the sham-operated group, peritoneum and skin were only incised and sutured.

All monoclonal antibodies were administered by intravenous injection once before the surgical operation. The sham-operated group was given vehicle. Both TBS139 and TBS182 were administered at various doses (10, 30, 100 mg/kg). Anti-mature TGF-beta antibody GC1008 (as described in U.S. Pat. No. 8,383,780) was used at 50 mg/kg as a positive control and anti-KLH antibody IC17 was used at 100 mg/kg as a negative control in this study. The animals were weighed and then killed by exsanguination under isoflurane anaesthesia on Day 7. Blood samples were collected from the heart cavity or the postcaval vein and maintained at −80 degrees C. until assayed. The kidney was quickly removed and weighed. Part of the kidney tissue was snap-frozen in liquid nitrogen or on dry ice for molecular analyses.

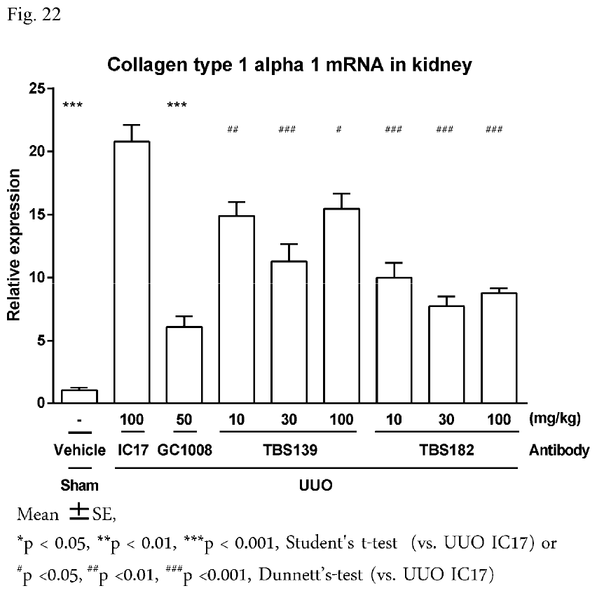

Total RNA was extracted from kidney tissues by the method explained in REFERENCE EXAMPLE 6-1. Mouse mitochondrial ribosomal protein L19 (MRPL19) was used as the endogenous reference for each sample, and relative mRNA expression values were calculated using double delta Ct analysis. The results of this experiment are shown in FIG. 22. For the antibodies, the inhibitory activity against kidney fibrosis was evaluated from the expression levels of collagen type 1 alpha 1 mRNA in kidney. UUO significantly increased the collagen mRNA level in the mice, and all three antibodies (GC1008, TBS139, and TBS182) reduced the level.

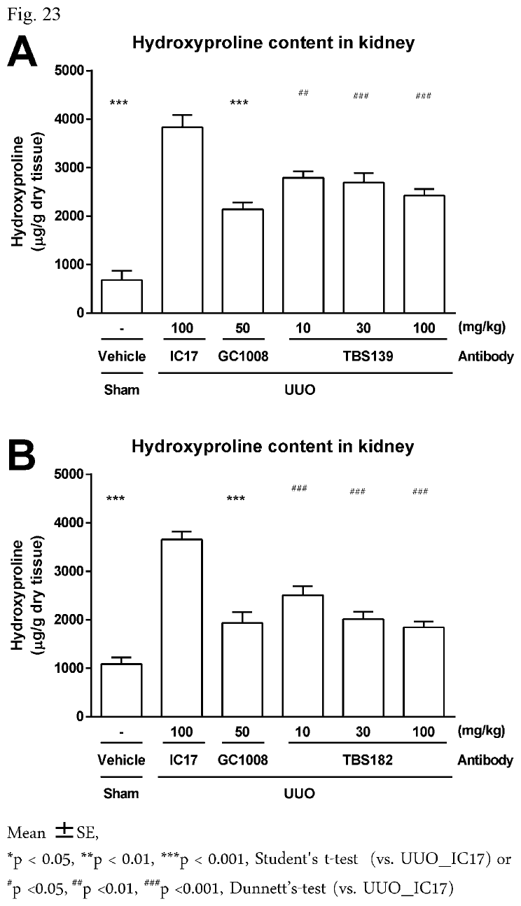

The content in kidney of hydroxyproline, which is one of the amino acids included in collagen, was measured to evaluate the extramatrical deposition in tissue. Wet kidney tissues were dried at 110 degrees C. for 3 hours and weighed. Then, 6N HCl (100 uL/1 mg dry tissue) was added to the dried tissue and boiled at 110 degrees C. for 3 hours. Samples were purified by filter, and 10 microliter of each sample was dispensed to a 96-well plate. The plate with samples was dried out at room temperature overnight and hydroxyproline was measured using a hydroxyproline assay kit (BioVision). The results of this experiment are shown in FIG. 23. A significant increase in hydroxyproline content was observed in disease-induced kidneys, and all antibodies (GC1008, TBS139, and TBS182) inhibited kidney fibrosis.

Data are presented as mean+/−standard error of the mean (SEM). Statistical analysis was performed using analysis of variance (ANOVA) and Student's t-test. P values of <0.05, <0.01, or <0.001 were considered significant.

### 3. In Vivo Efficacy of Anti-Latent TGF-Beta 1 Antibodies in a BLM-Induced Lung Fibrosis Mouse Model

The in vivo efficacy of monoclonal antibodies TBS139 and TBS182 was evaluated in a bleomycin (BLM)-induced lung fibrosis mouse model, which is characterized by the infiltration of leukocytes, fibroblast proliferation, and an increase in collagen within the lung tissue. All experimental animal care and handling were performed in accordance with the recommendations in the Guidelines for the Care and Use of Laboratory Animals at Chugai Pharmaceutical Co. Ltd, which is accredited by the Association for Assessment and Accreditation of Laboratory Animal Care International.

Specific-pathogen-free C57BL/6J male mice of 7 weeks of age were purchased from Japan Charles River Inc. (Kanagawa, Japan) and were acclimated for 1 week before the start of treatment. Animals were housed at 20-26 degrees C. with a 12:12 h light/dark cycle and fed with a commercial standard diet (#CE-2; CLEA Japan Inc., Shizuoka, Japan) and tap water ad libitum.

Intratracheal instillation of BLM (Nihon Kayaku, Tokyo, Japan) was performed at a dose of 1.5 mg/kg under isoflurane anesthesia. As a non-diseased control, saline was administered intratracheally. All monoclonal antibodies were administered by intravenous injection once before the BLM challenge. Both TBS139 and TBS182 were administered at two dose levels (10 and 50 mg/kg). Anti-mature TGF-beta antibody GC1008 (as described in U.S. Pat. No. 8,383,780) was used as a positive control and anti-KLH antibody IC17 was used as a negative control in this study (both at 50 mg/kg). The animals were weighed and then killed by exsanguination under isoflurane anaesthesia on Day 7. Blood samples were collected from the heart cavity or the postcaval vein and housed at −80 degrees C. until assayed. The lung was quickly removed and weighed. Part of the lung tissue was snap-frozen in liquid nitrogen or on dry ice for molecular analyses.

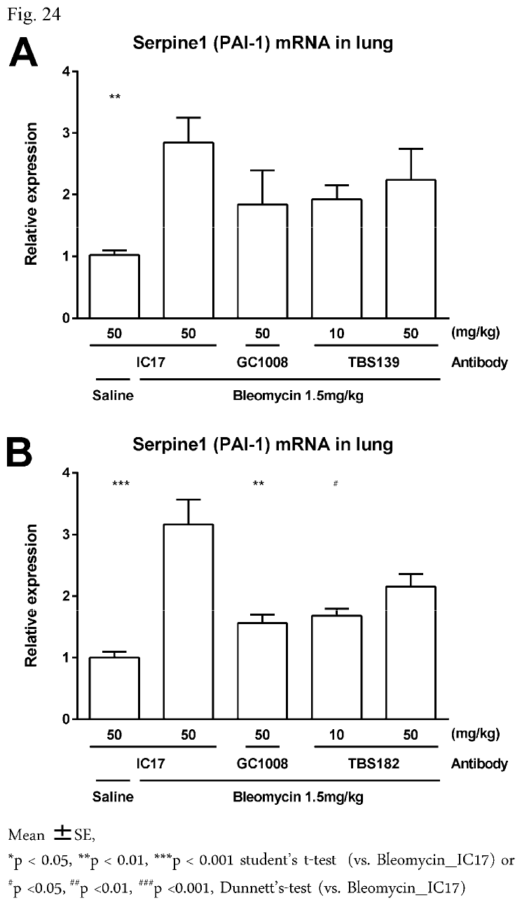

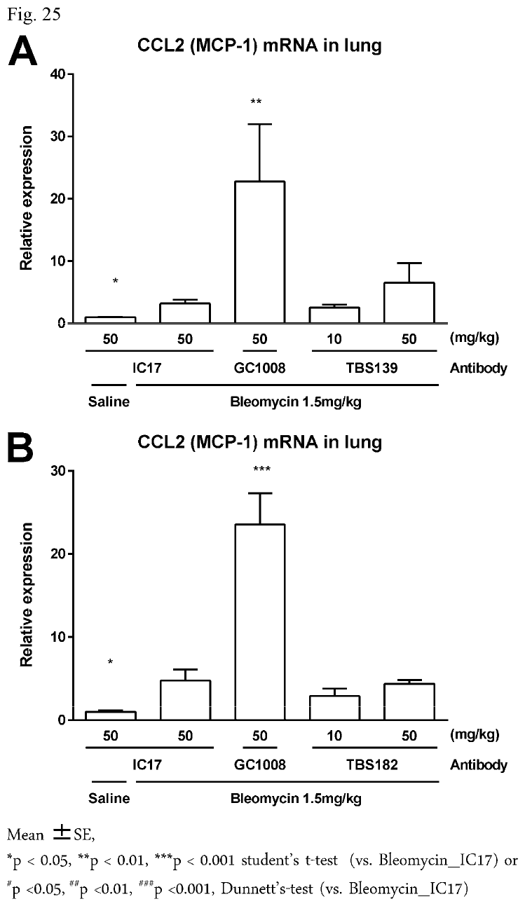

Total RNA extraction from lung tissues was performed by the method explained in REFERENCE EXAMPLE 6-1. Mouse glyceraldehyde 3-phosphate dehydrogenase (GAPDH) was used as the endogenous reference for each sample. Relative mRNA expression values were calculated using double delta Ct analysis. The results of this experiment are shown in FIGS. 24 and 25. For the antibodies, the inhibitory activity against lung fibrosis was evaluated from the expression levels of serpine 1 (PAI-1) mRNA in lung. BLM significantly increased the PAI-1 mRNA levels, and all antibodies (GC1008, TBS139, and TBS182) reduced the level. The inflammatory response was evaluated by measuring CCL2 (MCP-1) mRNA expression levels in lung. GC1008 dramatically enhanced the inflammatory response, but TBS139 and TBS182 did not significantly increase disease progression.

Data are presented as mean+/−standard error of the mean (SEM). Statistical analysis was performed using analysis of variance (ANOVA) and Student's t-test. P values of <0.05, <0.01, or <0.001 were considered significant.

### Reference Example 7: Toxicity Assay

Potential toxicity of anti-latent TGF-beta 1 neutralizing antibodies were confirmed and compared with that of anti-mature TGF-beta antibody in normal mice toxicity study.

GC1008 (anti-mature TGF-beta antibody), TBS139 and TBS182 (anti-latent TGF-beta 1 antibodies) were intravenously administered to 6-weeks-old female BALB/c mice (see Table 8) intermittently every two days for 5 weeks at a dose level of 50 mg/kg each. The control group received the vehicle (20 mmol/L histidine-HCl buffer containing 150 mmol/L NaCl, pH 6.0) alone. Clinical observations were conducted at least twice daily during the dosing period. Body weights were measured every three days for one week (i.e., on Days 1, 4 and 7). Blood samples for evaluation of blood chemistry, hematology and immunophenotyping were collected from all animals on Day 37. At the end of the dosing period, gross necropsy and histopathological examination was conducted in all animals.

TABLE 8

| | | Dose | | |
|---|---|---|---|---|
| | Dose level | concentration | Dose volume | Number of |
| Group | (mg/kg) | (mg/mL) | (mL/kg) | animals |
| Vehicle^{a)} | 0 | 0 | 5 | 6 |
| GC1008 | 50 | | | 12 |
| TBS139 | 50 | 10 | | 12 |
| TBS182 | 50 | | | 12 |

^{a)}20 mM Histidine-HCl 150 mM NaCl, pH 6.0

In the GC1008 group, three animals were found dead on Day 27, 28, and 35, respectively, and lower body weight and food consumption were noted.

Histopathologically, test article-related inflammatory changes, proliferative changes and effects to the extracellular matrix were observed. Major findings were as follows: Inflammatory changes in the aortic root/valve of the heart (4 animals), inflammatory changes in the lung (9 animals), in the esophagus (2 animals) and in the stomach (1 animal), cyst-like change in the tongue (4 animals), dysplastic change in the tooth (12 animals), hepatocyte change in the liver (3 animals), change in hair follicle in the skin/subcutis (4 animals) and hypoosteogenesis in the bone (femur/sternum, 12 animals). Dead animals showed severer changes, especially in the heart, than scheduled sacrifice animals. The cause of death was considered to be circulatory disturbance associated with the heart lesion such as hemorrhage/fibrinoid exudation in the aortic root/valve. Decrease in ALP (alkaline phosphatase) was observed and thought to be related to bone findings such as hypoosteogenetic change.

In the TBS139 and TBS182 groups, no mortality, changes of general condition, body weight, and food consumption were observed. On the other hand, test article related changes were histopathologically observed in TBS139/TBS182 as follows: Inflammatory cell infiltration and hemorrhage/fibrinoid exudation (1 animal), mesenchymal cell in aortic root/valve (2 animals) in the heart only in the TBS182 group, increase of inflammatory cell in the lung in TBS139 group (4 animals) and in the TBS182 group (7 animals), inflammatory cell infiltration in submucosa in the esophagus in the TBS139 group (1 animal) and in the TBS182 group (2 animals), hyperplasia of epithelium in the esophagus only in TBS182 (1 animal). The above changes were similar to those of the GC1008 group although severity and incidence of these findings were markedly lower than that in the GC1008 group. No test article-related changes in the tongue, stomach, tooth, liver, skin/subcutis and bone, which were observed in the GC1008 group, were noted in the TBS139 or TBS182 groups.

No toxicologically relevant test article-related changes were observed in hematology and immunophenotyping in all groups.

In conclusion, test article-related death, lower body weight and food consumption were noted in the GC1008 group. In addition, inflammatory changes, proliferative changes and effect for extracellular matrix were histopathologically observed in the heart, lung, esophagus, tongue, stomach, tooth, skin/subcutis, liver and bone (femur/sternum). The heart lesion was considered to be a cause of death similar to the previous study. In the TBS139 and TBS182 groups, no mortality, body weight change or food consumption change were noted. Histopathologically, similar changes to the GC1008 group were observed in the lung and esophagus in the TBS139 group without changes in the heart; and changes in the heart, lung and esophagus were observed in the TBS182 group. However severity and incidence of these findings in the TBS139 and TBS182 groups were markedly reduced compared to the GC1008 group.

Summary of main findings in 3 test articles as follows:

TABLE 9

| | | Group | | | |
|---|---|---|---|---|---|
| | | Vehicle | GC1008 | TBS139 | TBS182 |
| | | Dose(mg/kg) | | | |
| Organ/tissue | Findings | — | 50 | 50 | 50 |
| | | (n = 6) | (n = 12)^{a} | (n = 12) | (n = 12) |
| Heart | Inflammatory cell infiltration in aortic root/valve | 0 | 4 | 0 | 1 |
| | Hemorrhage/fibrinoid exudation in aortic root/valve | 0 | 4 | 0 | 1 |
| | Mesenchymal cell in aortic root/valve | 0 | 4 | 0 | 2 |
| Tooth | | (n = 6) | (n = 12)^{a} | (n = 12) | (n = 12) |
| | Dysplastic change | 0 | 12 | 0 | 0 |
| | Gigivitis | 0 | 5 | 0 | 0 |
| Tongue | | (n = 6) | (n = 12)^{a} | (n = 12) | (n = 12) |
| | Epithelial cystlike structure | 0 | 4 | 0 | 0 |
| Esophagus | | (n = 6) | (n = 12)^{a} | (n = 12) | (n = 12) |
| | inflammatory cell infiltration in submucosa | 0 | 2 | 1 | 2 |
| | Hyperplasia of epithelium | 0 | 0 | 0 | 1 |
| Lung | | (n = 6) | (n = 12)^{a} | (n = 12) | (n = 12) |
| | Increase of inflammatory cell | 0 | 9 | 4 | 7 |
| | Alveolar hemorrhage | 0 | 3 | 0 | 0 |
| Bone, femur | | (n = 6) | (n = 12)^{a} | (n = 12) | (n = 12) |
| | Increase of trabecular bone in metaphysis | 0 | 12 | 0 | 0 |
| | Decrease of osteoblast in metaphysis | 0 | 12 | 0 | 0 |
| | Decrease of osteoclast in metaphysis | 0 | 12 | 0 | 0 |
| | Retention of calcified caltilarge in metaphysis | 0 | 12 | 0 | 0 |
| Skin/subcutis | | (n = 6) | (n = 12)^{a} | (n = 12) | (n = 12) |
| | Increase of spindle cell around hair follicle | 0 | 4 | 0 | 0 |
| | Degeneration of hair follicle in anagen phase | 0 | 2 | 0 | 0 |

^{a}including 3 dead animals

### Reference Example 8: Identification of Anti-Human Latent TGF-Beta 1 Antibody

Additional anti-human latent TGF-beta 1 antibodies binding to cell surface TGF-beta 1 were prepared, selected, and assayed as follows:

Twelve to sixteen week old NZW rabbits were first immunized intradermally with human and mouse recombinant latent TGF-beta 1 proteins (100 microgram/dose/rabbit). Two weeks after the initial immunization, four more weekly doses were given, alternating between mouse and human recombinant latent TGF-beta 1 proteins (50 microgram/dose/rabbit). One week after the final immunization, spleen and blood from immunized rabbits were collected. Antigen-specific B-cells were stained, sorted and plated as described in Example 2. After cultivation, B-cell culture supernatants were collected for further analysis and cell pellets were cryopreserved.

A total of 3587 B cell supernatants were subjected to cell-based ELISA screening using FS293 cells overexpressing human or mouse latent TGF-beta 1 as described in BioTechniques 2003, 35:1014-1021.

A total of 94 B cells lines with binding to cell surface human latent TGF-beta 1, with or without binding to cell surface mouse latent TGF-beta 1 were selected for antibody gene cloning and downstream analysis. Anti-latent TGF-beta 1 antibodies from these 94 lines were cloned as described in REFERENCE EXAMPLE 2. The DNAs of their antibody heavy chain variable regions were amplified by reverse transcription PCR and recombined with the F1332m heavy chain constant region (SEQ ID NO: 36). The DNAs of their antibody light chain variable regions were amplified by reverse transcription PCR and recombined with the hkOMC light chain constant region (SEQ ID NO: 37). Recombinant antibodies were expressed transiently in FreeStyle™ 293-F cells according to the manufacturer's instructions (Life technologies) and purified using AssayMAP Bravo platform with protein A cartridge (Agilent) (TBA1235-TBA1328).

### Reference Example 9: Antibody Screening for Anti-Latent TGF-Beta 1 Antibody Generation

ELISA detecting mature TGF-beta 1 (Human TGF-beta 1 Quantikine ELISA Kit, R&D systems) was used to assess plasmin mediated TGF-beta 1 activation. Human recombinant latent TGF-beta 1 was incubated with human plasmin (Calbiochem), with or without the presence of anti-latent TGF-beta 1 antibodies at 37 degrees C. for 1 hour. The content of mature TGF-beta 1 in the mixture was analyzed by ELISA described above. The detection was done according to the manufacturer's procedure. Anti-latent TGF-beta 1 antibodies that have inhibitory activity against plasmin mediated TGF-beta 1 activation were screened out (e.g., TBA1277, TBA1300, and TBA1314). The amino acid sequences of the H chain and L chain of TBA1277 are shown in SEQ ID NOs: 70 and 71, respectively; the amino acid sequences of the H chain and L chain of TBA1300 are shown in SEQ ID NOs: 72 and 73, respectively; and the amino acid sequences of the H chain and L chain of TBA1314 are shown in SEQ ID NOs: 74 and 75, respectively. Further, the amino acid sequences of the variable regions (VRs) and CDRs (HVRs) of TBA1277, TBA1300, and TBA1314 are shown below.

TABLE 10

| Name | VR | Amino acid sequence | SEQ ID NO |
|---|---|---|---|
| TBA1277 | VH | QSVEESGGRLVTPGTPLTLTCTASGFSLSNYD | 76 |
| | | MSWVRQAPGKGLEWIGNIHTADTTSYARWA | |
| | | KGRFTISKTSTTVDLKITSPTSEDTATYFCARG | |
| | | GSDFFYYEPYNIWGPGTLVTVSS | |
| TBA1277 | VL | AYDMTQTPASVEAAVGGTVTVRCQASESIGS | 77 |
| | | RLAWYQQKVGQPPKLLIYDASTLASGVSSRF | |
| | | KGSGSGTEFTLTISDLECDDAATYYCQQYYTS | |
| | | NNVENTFGGGTKVEIK | |
| TBA1300 | VH | QEQLKESGGGLVTPGGTLTLTCTVSGFSLSNY | 78 |
| | | DMSWVRQAPGKGLEWIGAIYTAGTTSYASW | |
| | | AKGRFTISKTSTTVDLKITSPTTEDTATYFCAR | |
| | | GGSSFFYYAAHNIWGPGTLVTVSS | |
| TBA1300 | VL | AYDMTQTPASVEAAVGGTVTIKCQASQSIGS | 79 |
| | | NLAWYQQKPGQPPKLLIYDASKLPSGVPSRFK | |
| | | GSGSGTEFTLTISDLECDDAATYYCQQYYTTS | |
| | | DVDNTFGGGTKVEIK | |
| TBA1314 | VH | QSVEESGGRLVTPGTPLTLTCTVSGFSLSNYA | 80 |
| | | MTWVRQAPGKGLEWIGVIYAGGNTYYANW | |
| | | VNGRFTISKTSTTVDLKITSPTTEDTATYFCAR | |
| | | DVGSSSSGVYFNIWGPGTLVTVSS | |
| TBA1314 | VL | AYDMTQTPASVEVAVGGTVTIKCQASQSIGS | 81 |
| | | RLAWYQQKPGQPPKLLIYDASTLASGVSSRFK | |
| | | GSGSGTEFTLTISGVECADAATYYCQQDYSYR | |
| | | HVDNIEGGGTKVEIK | |

TABLE 11

| Name | HVR | Amino acid sequence | SEQ ID NO |
|---|---|---|---|
| TBA1277 | HVR H1 | NYDMS | 82 |
| TBA1277 | HVR H2 | NIHTADTTSYARWAKG | 83 |
| TBA1277 | HVR H3 | GGSDFFYYEPYNI | 84 |
| TBA1277 | HVR L1 | QASESIGSRLA | 85 |
| TBA1277 | HVR L2 | DASTLAS | 86 |
| TBA1277 | HVR L3 | QQYYTSNNVENT | 87 |
| TBA1300 | HVR H1 | NYDMS | 88 |
| TBA1300 | HVR H2 | AIYTAGTTSYASWAKG | 89 |
| TBA1300 | HVR H3 | GGSSFFTYAAHNI | 90 |
| TBA1300 | HVR L1 | QASQSIGSNLA | 91 |
| TBA1300 | HVR L2 | DASKLPS | 92 |
| TBA1300 | HVR L3 | QQYYTTSDVDNT | 93 |
| TBA1314 | HVR H1 | NYAMT | 94 |
| TBA1314 | HVR H2 | VIYAGGNTYYANWVNG | 95 |
| TBA1314 | HVR H3 | DVGSSSSGVYFNI | 96 |
| TBA1314 | HVR L1 | QASQSIGSRLA | 97 |
| TBA1314 | HVR L2 | DASTLAS | 98 |
| TBA1314 | HVR L3 | QQDYSYRHVDNI | 99 |

### Reference Example 10: Characterization of Anti-Latent TGF-Beta 1 Antibody

### 1. Anti-Latent TGF-Beta 1 Antibody Inhibition Against MMP2 and MMP9 Mediated Mouse Latent TGF-Beta 1 Activation

Mouse recombinant latent TGF-beta 1 was incubated with activated mouse MMP2 or mouse MMP9 (R&D systems), with or without the presence of anti-latent TGF-beta 1 antibodies at 37 degrees C. for 2 hours. MMP2 and MMP9 mediated mouse latent TGF-beta 1 activation and antibody mediated inhibition was analyzed by mature TGF-beta 1 ELISA (Human TGF-beta 1 Quantikine ELISA Kit, R&D systems) according to the manufacturer's procedure.

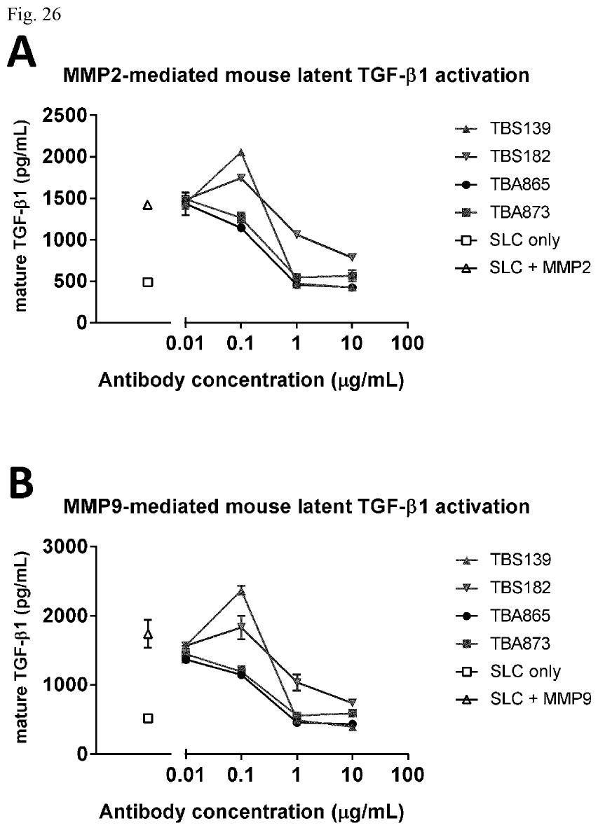

As shown in FIG. 26, MMP2 and MMP9 mediated mouse latent TGF-beta 1 activation was suppressed by the anti-latent TGF-beta 1 antibodies (TBS139, TBS182, TBA865, and TBA873).

### 2. Anti-Latent TGF-Beta 1 Antibody Inhibited Prekallikrein and Plasmin Mediated Human Latent TGF-Beta 1 Activation

Human recombinant latent TGF-beta 1 was incubated with human prekallikrein (Enzyme Research Laboratories) or plasmin (Calbiochem), with or without the presence of anti-latent TGF-beta 1 antibodies (TBA1277, TBA1300, or TBA1314) at 37 degrees C. for 1 hour. Prekallikrein and plasmin mediated TGF-beta 1 activation and antibody mediated inhibition was analyzed by mature TGF-beta 1 ELISA (Human TGF-beta 1 Quantikine ELISA Kit, R&D systems) according to manufacturer's procedure.

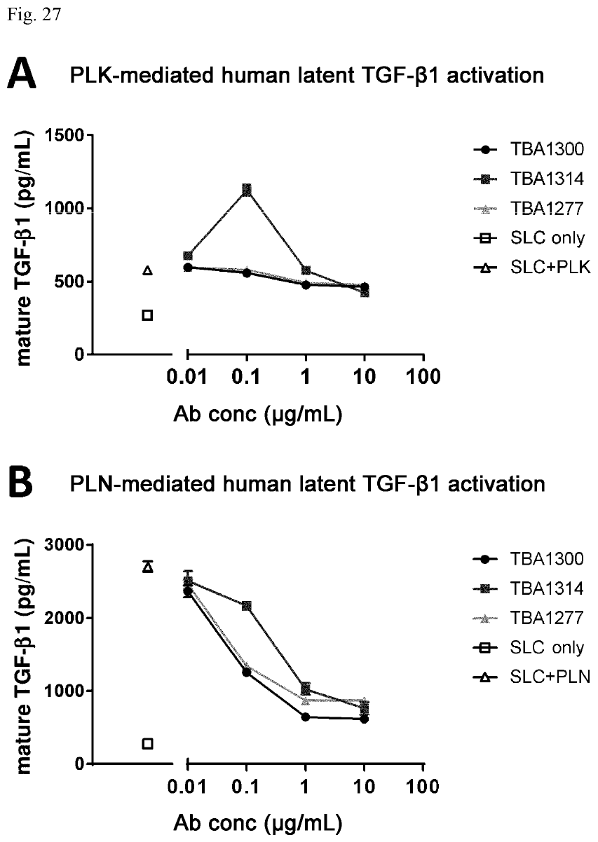

As shown in FIG. 27, prekallikrein and plasmin mediated human latent TGF-beta 1 activation was suppressed by anti-latent TGF-beta 1 antibodies (TBA1277, TBA1300, or TBA1314).

### 3. Inhibition of Plasmin Mediated TGF-Beta 1 Propeptide Cleavage by Anti-Latent TGF-Beta 1 Antibodies

Human recombinant latent TGF-beta 1 was incubated with human plasmin (Calbiochem), with or without the presence of anti-latent TGF-beta 1 antibodies (TBA1277, TBA1300, TBA1314, or TBA873) at 37 degrees C. for 1 hour. Anti-KLH antibody (IC17) was used for negative control. Camostat mesylate (TOCRIS) which is one of serine protease inhibitors was used for positive control. Mixed with 4×SDS-PAGE sample buffer (Wako), the samples were heated at 95 degrees C. for 5 minutes and then loaded for SDS gel electrophoresis. Proteins were transferred to a membrane by Trans-Blot® Turbo™ Transfer System (Bio-rad). Propeptide was detected using mouse anti-FLAG, M2-HRP antibody (Sigma-Aldrich). The membrane was incubated with ECL substrate, and images were taken by ImageQuant LAS4000 (GE Healthcare).

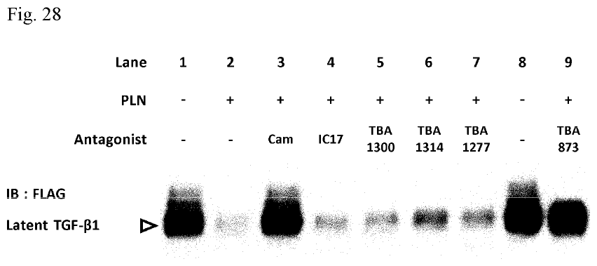

As shown in FIG. 28, the non-cleaved latent TGF-beta 1 (i.e., LAP region with FLAG-tag) was detected with the presence of TBA873 but not with the presence of TBA1277, TBA1300, and TBA1314, showing that propeptide cleavage by plasmin was only inhibited by TBA873 but not by TBA1277, TBA1300, and TBA1314.

### 4. Binding of Anti-Latent TGF-Beta 1 Antibodies to Large Latent TGF-Beta 1 Complex

Binding of anti-latent TGF-beta 1 antibodies to cell surface latent TGF-beta 1 was tested by FACS using Ba/F3 cells and FreeStyle™ 293-F cells (ThermoFisher), both of which endogenously express latent TGF-beta 1 forming LLC. Anti-latent TGF-beta 1 antibodies were incubated with each cell line for 30 minutes at 4 degrees C. and washed with FACS buffer (2% FBS, 2 mM EDTA in PBS). Anti-KLH antibody (IC17) was used for a negative control antibody. Goat F(ab′)2 anti-Human IgG, Mouse ads-PE (Southern Biotech, Cat. 2043-09) or Goat F(ab′)2 anti-Mouse IgG(H+L), Human Ads-PE (Southern Biotech, Cat. 1032-09) was then added and incubated for 30 minutes at 4 degrees C. and washed with FACS buffer. Data acquisition was performed on an FACS Verse (Becton Dickinson), followed by analysis using FlowJo software (Tree Star) and GraphPad Prism software (GraphPad).

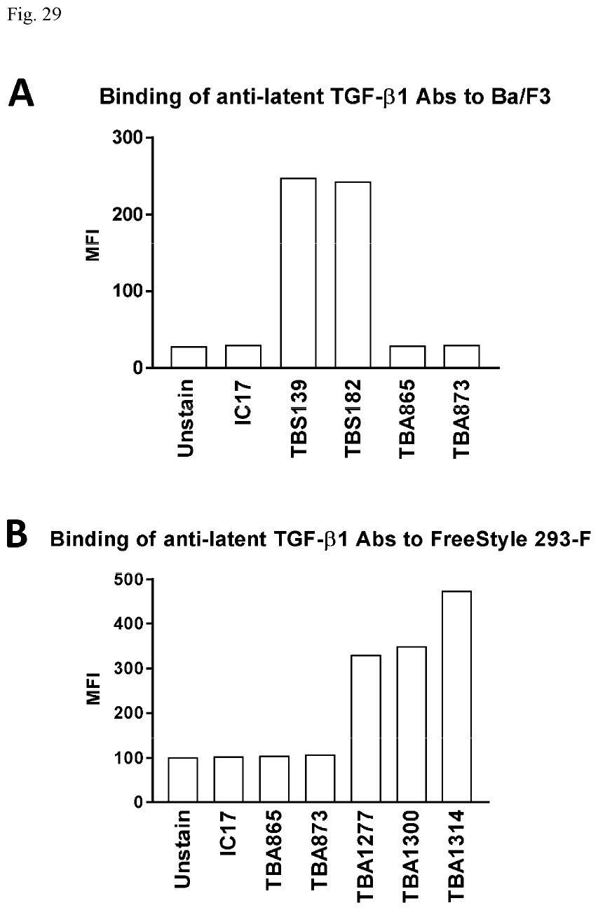

As shown in FIG. 29, TBS139 and TBS182 bind to Ba/F3 cells. TBA1277, TBA1300, and TBA1314 bind to FreeStyle™ 293-F cells.

### 5. Inhibition of MMP2 and MMP9 Mediated TGF-Beta 1 Propeptide Cleavage by Anti-Latent TGF-Beta 1 Antibodies

Mouse recombinant latent TGF-beta 1 was incubated with mouse MMP2 or mouse MMP9 (R&D systems), with or without the presence of anti-latent TGF-beta 1 antibodies (TBS139, TBS182, TBA865 or TBA873) at 37 degrees C. for 24 hour. Anti-KLH antibody (IC17) was used for negative control. GM6001 (TOCRIS) which is one of the MMP inhibitor was used for positive control. Mixed with 4×SDS-PAGE sample buffer (Wako), the samples were heated at 95 degrees C. for 5 minutes and then loaded for SDS gel electrophoresis. Proteins were transferred to membrane by Trans-Blot® Turbo™ Transfer System (Bio-rad). Propeptide was detected using mouse anti-FLAG, M2-HRP antibody (Sigma-Aldrich). The membrane was incubated with ECL substrate, and images were taken by ImageQuant LAS 4000 (GE Healthcare).

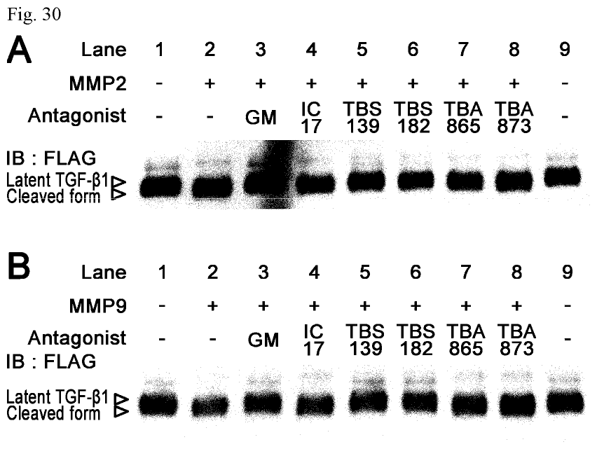

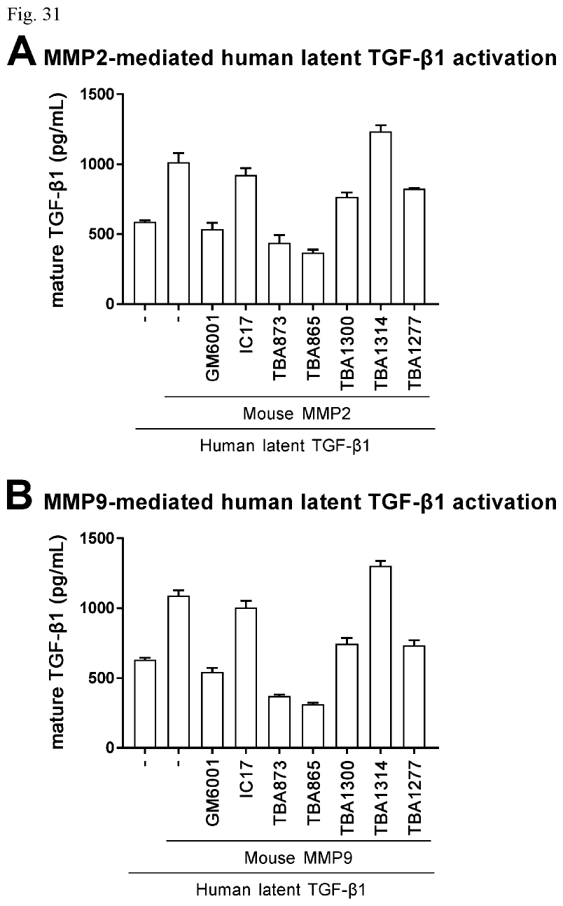

As shown in FIG. 30, the cleaved latent TGF-beta 1 (i.e., shortened LAP region of latent TGF-beta 1), whose band appears at the lower part of the image compared to the non-cleaved latent TGF-beta 1, was detected with the presence of TBA865 and TBA873, showing that propeptide cleavage by MMP2 (FIG. 30A) and MMP9 (FIG. 30B) was only inhibited by TBS139 and TBS182 but not by TBA865 and TBA873.

### 6. Inhibition Against MMP2 and MMP9 Mediated Human Latent TGF-Beta 1 Activation by Anti-Latent TGF-Beta 1 Antibodies

Human recombinant latent TGF-beta 1 was incubated with activated mouse MMP2 or mouse MMP9 (R&D systems), with or without the presence of anti-latent TGF-beta 1 antibodies at 37 degrees C. for 2 hours. MMP2 and MMP9 mediated human latent TGF-beta 1 activation and antibody mediated inhibition was analyzed by mature TGF-beta 1 ELISA (Human TGF-beta 1 Quantikine ELISA Kit, R&D systems) according to the manufacturer's procedure.

As shown in FIG. 31, MMP2 and MMP9 mediated human latent TGF-beta 1 activation was suppressed by the anti-latent TGF-beta 1 antibodies (TBA865, TBA873, TBA1300, and TBA1277).

### 7. Biacore Analysis for Binding Affinity Evaluation of Anti-Latent TGF-Beta 1 Antibodies

The affinities of anti-latent TGF-beta 1 antibodies binding to human latent TGF-beta 1 at pH 7.4 were determined at 37 degrees C. using Biacore T200 instrument (GE Healthcare). Anti-human Fc (GE Healthcare) was immobilized onto all flow cells of a CM4 sensor chip using an amine coupling kit (GE Healthcare). All antibodies and analytes were prepared in ACES pH 7.4 containing 20 mM ACES, 150 mM NaCl, 1.2 mM CaCl₂, 0.05% Tween 20, and 0.005% NaN₃. Each antibody was captured onto the sensor surface by anti-human Fc. Antibody capture levels were aimed at 300 resonance unit (RU). Recombinant human latent TGF-beta 1 was injected at a concentration of 12.5 to 200 nM prepared by two-fold serial dilution, followed by dissociation. Sensor surface was regenerated each cycle with 3M MgCl₂. Binding affinity was determined by processing and fitting the data to 1:1 binding model using Biacore T200 Evaluation software, version 2.0 (GE Healthcare). The affinities of anti-latent TGF-beta 1 antibodies binding to human latent TGF-beta 1 are shown in Table 12.

TABLE 12

| | Human latent TGF-beta 1 | | |
|---|---|---|---|
| Ab name | ka (M−1s−1) | kd (s−1) | KD (M) |
| TBA1300ff-F1332m | 5.93E+05 | 2.31E−05 | 3.89E−11 |
| TBA1314aa-F1332m | 4.02E+05 | 2.97E−06 * | 7.38E−12 |
| TBA1277cc-F1332m | 8.67E+05 | 1.63E−05 | 1.88E−11 |

* slow off rate <1E−05, K.D cannot be uniquely determined.

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

1. An anti-latent TGF-beta 1 antibody, which comprises:
(1) HVR-H1, HVR-H2 and HVR-H3 comprising the amino acid sequences of SEQ ID NOs: 5, 6, 7, respectively, and HVR-L1, HVR-L2 and HVR-L3 comprising the amino acid sequences of SEQ ID NOs: 8, 9, 10, respectively;
(2) HVR-H1, HVR-H2 and HVR-H3 comprising the amino acid sequences of SEQ ID NOs: 11, 12, 13, respectively, and HVR-L1, HVR-L2 and HVR-L3 comprising the amino acid sequences of SEQ ID NOs: 14, 15, 16, respectively; or
(3) HVR-H1, HVR-H2 and HVR-H3 comprising the amino acid sequences of SEQ ID NOs: 17, 18, 19, respectively, and HVR-L1, HVR-L2 and HVR-L3 comprising the amino acid sequences of SEQ ID NOs: 20, 21, 22, respectively.

2. An anti-latent TGF-beta 1 antibody that binds to the same epitope as a reference antibody, wherein the reference antibody comprises:
(1) HVR-H1, HVR-H2 and HVR-H3 comprising the amino acid sequences of SEQ ID NOs: 5, 6, 7, respectively, and HVR-L1, HVR-L2 and HVR-L3 comprising the amino acid sequences of SEQ ID NOs: 8, 9, 10, respectively;
(2) HVR-H1, HVR-H2 and HVR-H3 comprising the amino acid sequences of SEQ ID NOs: 11, 12, 13, respectively, and HVR-L1, HVR-L2 and HVR-L3 comprising the amino acid sequences of SEQ ID NOs: 14, 15, 16, respectively; or
(3) HVR-H1, HVR-H2 and HVR-H3 comprising the amino acid sequences of SEQ ID NOs: 17, 18, 19, respectively, and HVR-L1, HVR-L2 and HVR-L3 comprising the amino acid sequences of SEQ ID NOs: 20, 21, 22, respectively.

3. The anti-latent TGF-beta 1 antibody according to claim 2, wherein the antibody binds to human latent TGF-beta 1 and mouse latent TGF-beta 1.

4. The anti-latent TGF-beta 1 antibody according to claim 2 or 3, wherein the antibody binds to latent TGF-beta 1 forming cell surface latent TGF beta 1, large latent complex (LLC), and/or small latent complex (SLC).

5. The anti-latent TGF-beta 1 antibody according to any one of claims 2 to 4, wherein the antibody binds to the latency-associated peptide (LAP) region of latent TGF-beta 1.

6. The anti-latent TGF-beta 1 antibody according to any one of claims 2 to 5, where in the antibody does not bind to mature TGF-beta 1.

7. The anti-latent TGF-beta 1 antibody according to any one of claims 2 to 6, wherein the antibody inhibits a protease mediated activation of latent TGF-beta 1 without inhibiting a protease mediated cleavage of the LAP region of latent TGF-beta 1.

8. The anti-latent TGF-beta 1 antibody according to claim 7, wherein the protease is selected from the group consisting of plasmin (PLN), plasma kallikrein (PLK), matrix metalloproteinase (MMP) 2 and MMP9.

9. The anti-latent TGF-beta 1 antibody according to any one of claims 1 to 8, wherein the antibody does not inhibit integrin mediated activation of latent TGF-beta 1.

10. The anti-latent TGF-beta 1 antibody according to any one of claims 1 to 9, wherein the antibody is a human, humanized or chimeric antibody.

11. The anti-latent TGF-beta 1 antibody according to any one of claims 1 to 9, wherein the antibody comprises an Fc region with reduced binding activity towards an Fc gamma receptor.

12. An isolated nucleic acid encoding the antibody of any one of claims 1 to 11.

13. A host cell comprising the nucleic acid of claim 12.

14. A method of producing an antibody comprising culturing the host cell of claim 13 so that the antibody is produced.

15. A pharmaceutical composition comprising the anti-latent TGF-beta 1 antibody according to any one of claims 1 to 11 and a pharmaceutically acceptable carrier.


SEQUENCE LISTING (99 sequences)

SEQ ID NO: 1 - protein, 390 aa, Homo sapiens
MPPSGLRLLPLLLPLLWLLVLTPGRPAAGLSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 2 - DNA, 2741 bp, Homo sapiens
  source 1..2741: mol_type = unassigned DNA
acctccctccgcggagcagccagacagcgagggccccggccgggggcaggggggacgccccgtccggggcacccccccggctctgagccgcccgcggggccggcctcggcccggagcggaggaaggagtcgccgaggagcagcctgaggccccagagtctgagacgagccgccgccgcccccgccactgcggggaggagggggaggaggagcgggaggagggacgagctggtcgggagaagaggaaaaaaacttttgagacttttccgttgccgctgggagccggaggcgcggggacctcttggcgcgacgctgccccgcgaggaggcaggacttggggaccccagaccgcctccctttgccgccggggacgcttgctccctccctgccccctacacggcgtccctcaggcgcccccattccggaccagccctcgggagtcgccgacccggcctcccgcaaagacttttccccagacctcgggcgcaccccctgcacgccgccttcatccccggcctgtctcctgagcccccgcgcatcctagaccctttctcctccaggagacggatctctctccgacctgccacagatcccctattcaagaccacccaccttctggtaccagatcgcgcccatctaggttatttccgtgggatactgagacacccccggtccaagcctcccctccaccactgcgcccttctccctgaggacctcagctttccctcgaggccctcctaccttttgccgggagacccccagcccctgcaggggcggggcctccccaccacaccagccctgttcgcgctctcggcagtgccggggggcgccgcctcccccatgccgccctccgggctgcggctgctgccgctgctgctaccgctgctgtggctactggtgctgacgcctggccggccggccgcgggactatccacctgcaagactatcgacatggagctggtgaagcggaagcgcatcgaggccatccgcggccagatcctgtccaagctgcggctcgccagccccccgagccagggggaggtgccgcccggcccgctgcccgaggccgtgctcgccctgtacaacagcacccgcgaccgggtggccggggagagtgcagaaccggagcccgagcctgaggccgactactacgccaaggaggtcacccgcgtgctaatggtggaaacccacaacgaaatctatgacaagttcaagcagagtacacacagcatatatatgttcttcaacacatcagagctccgagaagcggtacctgaacccgtgttgctctcccgggcagagctgcgtctgctgaggctcaagttaaaagtggagcagcacgtggagctgtaccagaaatacagcaacaattcctggcgatacctcagcaaccggctgctggcacccagcgactcgccagagtggttatcttttgatgtcaccggagttgtgcggcagtggttgagccgtggaggggaaattgagggctttcgccttagcgcccactgctcctgtgacagcagggataacacactgcaagtggacatcaacgggttcactaccggccgccgaggtgacctggccaccattcatggcatgaaccggcctttcctgcttctcatggccaccccgctggagagggcccagcatctgcaaagctcccggcaccgccgagccctggacaccaactattgcttcagctccacggagaagaactgctgcgtgcggcagctgtacattgacttccgcaaggacctcggctggaagtggatccacgagcccaagggctaccatgccaacttctgcctcgggccctgcccctacatttggagcctggacacgcagtacagcaaggtcctggccctgtacaaccagcataacccgggcgcctcggcggcgccgtgctgcgtgccgcaggcgctggagccgctgcccatcgtgtactacgtgggccgcaagcccaaggtggagcagctgtccaacatgatcgtgcgctcctgcaagtgcagctgaggtcccgccccgccccgccccgccccggcaggcccggccccaccccgccccgcccccgctgccttgcccatgggggctgtatttaaggacacccgtgccccaagcccacctggggccccattaaagatggagagaggactgcggatctctgtgtcattgggcgcctgcctggggtctccatccctgacgttcccccactcccactccctctctctccctctctgcctcctcctgcctgtctgcactattcctttgcccggcatcaaggcacaggggaccagtggggaacactactgtagttagatctatttattgagcaccttgggcactgttgaagtgccttacattaatgaactcattcagtcaccatagcaacactctgagatgcagggactctgataacacccattttaaaggtgaggaaacaagcccagagaggttaagggaggagttcctgcccaccaggaacctgctttagtgggggatagtgaagaagacaataaaagatagtagttcaggccaggcggggtggctcacgcctgtaatcctagcacttttgggaggcagagatgggaggattacttgaatccaggcatttgagaccagcctgggtaacatagtgagaccctatctctacaaaacacttttaaaaaatgtacacctgtggtcccagctactctggaggctaaggtgggaggatcacttgatcctgggaggtcaaggctgcag

SEQ ID NO: 3 - protein, 390 aa, Mus musculus
MPPSGLRLLPLLLPLPWLLVLTPGRPAAGLSTCKTIDMELVKRKRIEAIRGQILSKLRLASPPSQGEVPPGPLPEAVLALYNSTRDRVAGESADPEPEPEADYYAKEVTRVLMVDRNNAIYEKTKDISHSIYMFFNTSDIREAVPEPPLLSRAELRLQRLKSSVEQHVELYQKYSNNSWRYLGNRLLTPTDTPEWLSFDVTGVVRQWLNQGDGIQGFRFSAHCSCDSKDNKLHVEINGISPKRRGDLGTIHDMNRPFLLLMATPLERAQHLHSSRHRRALDTNYCFSSTEKNCCVRQLYIDFRKDLGWKWIHEPKGYHANFCLGPCPYIWSLDTQYSKVLALYNQHNPGASASPCCVPQALEPLPIVYYVGRKPKVEQLSNMIVRSCKCS

SEQ ID NO: 4 - DNA, 2191 bp, Mus musculus
  source 1..2191: mol_type = unassigned DNA
cgccgccgccgccgcccttcgcgccccaggccgtccccctcctcctcccgccgcggatcctccagacagccaggcccccggccggggcaggggggacgccccttcggggcacccccggctctgagccgcactcggagtcggcctccgctgggagccggcaaaggagcagccgaggagccgtccgaggccccagagtctgagaccagccgccgccgcagggaggagggggaggaggagtgggaggagggacgagctggttgagagaagaggaaaaaagttttgagacttttccgctgctactgcaagtcagagacgtggggacttcttggcactgcgctgtctcgcaaggaggcaggacctgaggactccagacagccctgctcaccgtcgtggacactcgatcgctacccggcgttcctcagacgcccctattccggaccagccctcgggagccacaaaccccgcctcccgcgaagacttcaccccaaagctggggcgcaccccttgcacgccgccctccccccagcctgcctcttgagtccctcgcatcccaggaccctctctcccccgagaggcagatctccctcggacctgctggcagtagctcccctatttaagaacacccacttttggatctcagagagcgctcatctcgatttttaccctggtggtatactgagacaccttggtgtcagagcctcaccgcgactcctgctgctttctccctcaacctcaaattattcaggactatcacctacctttccttgggagaccccaccccacaagccctgcaggggcggggcctccgcatcccacctttgccgagggttcccgctctccgaagtgccgtggggcgccgcctcccccatgccgccctcggggctgcggctactgccgcttctgctcccactcccgtggcttctagtgctgacgcccgggaggccagccgcgggactctccacctgcaagaccatcgacatggagctggtgaaacggaagcgcatcgaagccatccgtggccagatcctgtccaaactaaggctcgccagtcccccaagccagggggaggtaccgcccggcccgctgcccgaggcggtgctcgctttgtacaacagcacccgcgaccgggtggcaggcgagagcgccgacccagagccggagcccgaagcggactactatgctaaagaggtcacccgcgtgctaatggtggaccgcaacaacgccatctatgagaaaaccaaagacatctcacacagtatatatatgttcttcaatacgtcagacattcgggaagcagtgcccgaacccccattgctgtcccgtgcagagctgcgcttgcagagattaaaatcaagtgtggagcaacatgtggaactctaccagaaatatagcaacaattcctggcgttaccttggtaaccggctgctgacccccactgatacgcctgagtggctgtcttttgacgtcactggagttgtacggcagtggctgaaccaaggagacggaatacagggctttcgattcagcgctcactgctcttgtgacagcaaagataacaaactccacgtggaaatcaacgggatcagccccaaacgtcggggcgacctgggcaccatccatgacatgaaccggcccttcctgctcctcatggccacccccctggaaagggcccagcacctgcacagctcacggcaccggagagccctggataccaactattgcttcagctccacagagaagaactgctgtgtgcggcagctgtacattgactttaggaaggacctgggttggaagtggatccacgagcccaagggctaccatgccaacttctgtctgggaccctgcccctatatttggagcctggacacacagtacagcaaggtccttgccctctacaaccaacacaacccgggcgcttcggcgtcaccgtgctgcgtgccgcaggctttggagccactgcccatcgtctactacgtgggtcgcaagcccaaggtggagcagttgtccaacatgattgtgcgctcctgcaagtgcagctgaagccccgccccgccccgcccctcccggcaggcccggccccgcccccgccccgcccgcagcgcccggggctgtatttaaggacacctgcaccccccctcaagcccacctggggggcccattaaaggtgacagaggaaaaaaaaaaaaaaaaa

SEQ ID NO: 5 - protein, 5 aa, Oryctolagus cuniculus
NYYMN

SEQ ID NO: 6 - protein, 16 aa, Oryctolagus cuniculus
LTDNDGTTYYANWAKG

SEQ ID NO: 7 - protein, 8 aa, Oryctolagus cuniculus
DAGYSRNL

SEQ ID NO: 8 - protein, 11 aa, Oryctolagus cuniculus
QASQSISSSLA

SEQ ID NO: 9 - protein, 7 aa, Oryctolagus cuniculus
RASTLAS

SEQ ID NO: 10 - protein, 12 aa, Oryctolagus cuniculus
QQTYSWSGLDNV

SEQ ID NO: 11 - protein, 5 aa, Oryctolagus cuniculus
SYAMN

SEQ ID NO: 12 - protein, 16 aa, Oryctolagus cuniculus
YIYTSGTTYHANWARG

SEQ ID NO: 13 - protein, 14 aa, Oryctolagus cuniculus
GTGISDYYYWVMDL

SEQ ID NO: 14 - protein, 11 aa, Oryctolagus cuniculus
QASQSISTYLA

SEQ ID NO: 15 - protein, 7 aa, Oryctolagus cuniculus
KASTLAS

SEQ ID NO: 16 - protein, 10 aa, Oryctolagus cuniculus
QSYCDGDSVG

SEQ ID NO: 17 - protein, 5 aa, Oryctolagus cuniculus
NYAMG

SEQ ID NO: 18 - protein, 16 aa, Oryctolagus cuniculus
IISSGGVSYYASWARG

SEQ ID NO: 19 - protein, 15 aa, Oryctolagus cuniculus
GQVPSSDTYYVPFNL

SEQ ID NO: 20 - protein, 11 aa, Oryctolagus cuniculus
QASQSIGSDLA

SEQ ID NO: 21 - protein, 7 aa, Oryctolagus cuniculus
RASTLAS

SEQ ID NO: 22 - protein, 12 aa, Oryctolagus cuniculus
QTYYFTISSYGA

SEQ ID NO: 23 - protein, 113 aa, Oryctolagus cuniculus
QSLEESGGRLVTPGTPLTLTCTTSGFSLNNYYMNWVRQAPGKGLEWIGLTDNDGTTYYANWAKGRFTVSKTSTTVDLKMTSPTTEDTATYFCVRDAGYSRNLWGPGTLVTVSS

SEQ ID NO: 24 - protein, 110 aa, Oryctolagus cuniculus
AYDMTQTPASVEVAVGGTVTIKCQASQSISSSLAWYQQKPGQPPKQLIYRASTLASGVSSRFKGSGSGTQFTLTISGVECADAATYYCQQTYSWSGLDNVFGGGTKVEIK

SEQ ID NO: 25 - protein, 119 aa, Oryctolagus cuniculus
QSVEESGGRLVTPGTPLTLTCTVSGFSLSSYAMNWVRQAPGKGLEWIGYIYTSGTTYHANWARGRFTISKTSTTVDLKMTSLTTEDTATYFCARGTGISDYYYWVMDLWGPGTLVTVSS

SEQ ID NO: 26 - protein, 108 aa, Oryctolagus cuniculus
AFELTQTPSSVEAAVGGTVTIKCQASQSISTYLAWYQQRPGQPPKLLIYKASTLASGVPSRFKGSGSGTDFTLTISDLECADAATYFCQSYCDGDSVGFGGGTKVEIK

SEQ ID NO: 27 - protein, 120 aa, Oryctolagus cuniculus
QSVEESGGRLVTPGTPLTLTCTVSGFSLSNYAMGWVRQAPGKGLEWIGIISSGGVSYYASWARGRFTISKTSTTVDLKITSPTPEDTGTYFCARGQVPSSDTYYVPFNLWGPGTLVTVSS

SEQ ID NO: 28 - protein, 110 aa, Oryctolagus cuniculus
DIAMTQTPASVEAAVGGTVTIKCQASQSIGSDLAWYQQKPGQPPKLLIYRASTLASGVPLRFKGSGSGTQFTLTISDLECADAATYYCQTYYFTISSYGAFGGGTKVEIK

SEQ ID NO: 29 - protein, 387 aa, Homo sapiens
MKWVTFLLLLFISGSAFSDYKDDDDKLSTSKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 30 - DNA, 1167 bp, Homo sapiens
  source 1..1167: mol_type = unassigned DNA
atgaagtgggtaacctttctcctcctcctcttcatctccggttctgccttttccgactacaaggatgacgatgacaagctatccacctccaagactatcgacatggagctggtgaagcggaagcgcatcgaggccatccgcggccagatcctgtccaagctgcggctcgccagccccccgagccagggggaggtgccgcccggcccgctgcccgaggccgtgctcgccctgtacaacagcacccgcgaccgggtggccggggagagtgcagaaccggagcccgagcctgaggccgactactacgccaaggaggtcacccgcgtgctaatggtggaaacccacaacgaaatctatgacaagttcaagcagagtacacacagcatatatatgttcttcaacacatcagagctccgagaagcggtacctgaacccgtgttgctctcccgggcagagctgcgtctgctgaggctcaagttaaaagtggagcagcacgtggagctgtaccagaaatacagcaacaattcctggcgatacctcagcaaccggctgctggcacccagcgactcgccagagtggttatcttttgatgtcaccggagttgtgcggcagtggttgagccgtggaggggaaattgagggctttcgccttagcgcccactgctcctgtgacagcagggataacacactgcaagtggacatcaacgggttcactaccggccgccgaggtgacctggccaccattcatggcatgaaccggcctttcctgcttctcatggccaccccgctggagagggcccagcatctgcaaagctcccggcaccgccgagccctggacaccaactattgcttcagctccacggagaagaactgctgcgtgcggcagctgtacattgacttccgcaaggacctcggctggaagtggatccacgagcccaagggctaccatgccaacttctgcctcgggccctgcccctacatttggagcctggacacgcagtacagcaaggtcctggccctgtacaaccagcataacccgggcgcctcggcggcgccgtgctgcgtgccgcaggcgctggagccgctgcccatcgtgtactacgtgggccgcaagcccaaggtggagcagctgtccaacatgatcgtgcgctcctgcaagtgcagctgatga

SEQ ID NO: 31 - protein, 387 aa, Mus musculus
MKWVTFLLLLFISGSAFSDYKDDDDKLSTSKTIDMELVKRKRIEAIRGQILSKLRLASPPSQGEVPPGPLPEAVLALYNSTRDRVAGESADPEPEPEADYYAKEVTRVLMVDRNNAIYEKTKDISHSIYMFFNTSDIREAVPEPPLLSRAELRLQRLKSSVEQHVELYQKYSNNSWRYLGNRLLTPTDTPEWLSFDVTGVVRQWLNQGDGIQGFRFSAHCSCDSKDNKLHVEINGISPKRRGDLGTIHDMNRPFLLLMATPLERAQHLHSSRHRRALDTNYCFSSTEKNCCVRQLYIDFRKDLGWKWIHEPKGYHANFCLGPCPYIWSLDTQYSKVLALYNQHNPGASASPCCVPQALEPLPIVYYVGRKPKVEQLSNMIVRSCKCS

SEQ ID NO: 32 - DNA, 1167 bp, Mus musculus
  source 1..1167: mol_type = unassigned DNA
atgaagtgggtaacctttctcctcctcctcttcatctccggttctgccttttccgactacaaggatgacgatgacaagctctccacctccaagaccatcgacatggagctggtgaaacggaagcgcatcgaagccatccgtggccagatcctgtccaaactaaggctcgccagtcccccaagccagggggaggtaccgcccggcccgctgcccgaggcggtgctcgctttgtacaacagcacccgcgaccgggtggcaggcgagagcgccgacccagagccggagcccgaagcggactactatgctaaagaggtcacccgcgtgctaatggtggaccgcaacaacgccatctatgagaaaaccaaagacatctcacacagtatatatatgttcttcaatacgtcagacattcgggaagcagtgcccgaacccccattgctgtcccgtgcagagctgcgcttgcagagattaaaatcaagtgtggagcaacatgtggaactctaccagaaatatagcaacaattcctggcgttaccttggtaaccggctgctgacccccactgatacgcctgagtggctgtcttttgacgtcactggagttgtacggcagtggctgaaccaaggagacggaatacagggctttcgattcagcgctcactgctcttgtgacagcaaagataacaaactccacgtggaaatcaacgggatcagccccaaacgtcggggcgacctgggcaccatccatgacatgaaccggcccttcctgctcctcatggccacccccctggaaagggcccagcacctgcacagctcacggcaccggagagccctggataccaactattgcttcagctccacagagaagaactgctgtgtgcggcagctgtacattgactttaggaaggacctgggttggaagtggatccacgagcccaagggctaccatgccaacttctgtctgggaccctgcccctatatttggagcctggacacacagtacagcaaggtccttgccctctacaaccaacacaacccgggcgcttcggcgtcaccgtgctgcgtgccgcaggctttggagccactgcccatcgtctactacgtgggtcgcaagcccaaggtggagcagttgtccaacatgattgtgcgctcctgcaagtgcagctgatga

SEQ ID NO: 33 - DNA, 1825 bp, Rattus norvegicus
  source 1..1825: mol_type = unassigned DNA
atgaagtgggtaacctttctcctcctcctcttcatctccggttctgccttttccaggggtgtgtttcgccgagaagcacacaagagtgagatcgcccatcggtttaaggacttgggagaacagcatttcaaaggcctagtcctgattgccttttcccagtatctccagaaatgcccatatgaagagcatatcaaattggtgcaggaagtaacagactttgcaaaaacatgtgtcgctgatgagaatgccgaaaactgtgacaagtccattcacactctcttcggagacaagttatgcgccattccaaagcttcgcgacaactacggtgaactggctgactgctgtgcaaaacaagagcccgaaagaaacgagtgtttcctgcagcacaaggatgacaaccccaacctgccacccttccagaggccggaggctgaggccatgtgcacctccttccaggagaaccctaccagctttctgggacactatttgcatgaagttgccaggagacatccttatttctatgccccagaactcctttactatgctgagaaatacaatgaggttctgacccagtgctgcacagagtctgacaaagcagcctgcctgacaccgaagcttgatgccgtgaaagagaaagcactggtcgcagctgtccgtcagaggatgaagtgctccagtatgcagagatttggagagagagccttcaaagcctgggcagtagctcgtatgagccagcgattccccaatgctgagttcgcagaaatcaccaaattggcaacagacctcaccaaaatcaacaaggagtgctgtcacggcgacctgttggaatgcgcggatgacagggcggaacttgccaagtacatgtgtgagaaccaggccactatctccagcaaactgcaggcttgctgtgataagccagtgctgcagaaatcccagtgtctcgctgagatagaacatgacaacattcctgccgatctgccctcaatagctgctgactttgttgaggataaggaagtgtgtaagaactatgctgaggccaaggatgtcttcctgggcacgtttttgtatgaatattcaagaaggcaccccgattactccgtgtccctgctgctgagacttgctaagaaatatgaagccacactggagaagtgctgtgctgaaggcgatcctcctgcctgctacggcacagtgcttgcagaatttcagcctcttgtagaagaacctaagaacttggtcaaaactaactgtgagctttacgagaagcttggagagtatggattccaaaacgccattctggttcgatacacccagaaagcacctcaggtgtcgaccccaactctcgtggaggcagcaagaaacctgggaagagtgggcaccaagtgttgtacccttcctgaagctcagagactgccctgtgtggaagactatctgtctgccatcctgaaccgtctgtgtgtgctgcatgagaagaccccagtgagcgagaaggtcaccaagtgctgtagtgggtccctggtggaaagacggccatgtttctctgctctgacagttgacgagacatatgtccccaaagagtttaaagctgagaccttcaccttccactctgatatctgcacactcccagacaaggagaagcagataaagaagcaaacggctctcgctgagctggtgaaacacaagcccaaggccacagaagatcagctgaagacggtgatgggtgacttcgcacaattcgtggacaagtgttgcaaggctgccgacaaggataactgcttcgccactgaggggccaaaccttgttgctagaagcaaagaagccttagcct

SEQ ID NO: 34 - protein, 328 aa, synthetic construct
  REGION 1..328: note = an artificially synthesized sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELRRGPKVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 35 - protein, 107 aa, synthetic construct
  REGION 1..107: note = an artificially synthesized sequence
RTVAAPSVFIFPPSDEQLKSGTASVVCLLNNFYPREAKVQWKVDNALQSGNSQESVTEQDSKDCTYSLSSTLTLSKADYEKHKVYACEVTHQGLSSPVTKSFNRGEC

SEQ ID NO: 36 - protein, 441 aa, synthetic construct
  REGION 1..441: note = artificially synthesized
QSLEESGGRLVTPGTPLTLTCTTSGFSLNNYYMNWVRQAPGKGLEWIGLTDNDGTTYYANWAKGRFTVSKTSTTVDLKMTSPTTEDTATYFCVRDAGYSRNLWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELRRGPKVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 37 - protein, 217 aa, synthetic construct
  REGION 1..217: note = artificially synthesized
AYDMTQTPASVEVAVGGTVTIKCQASQSISSSLAWYQQKPGQPPKQLIYRASTLASGVSSRFKGSGSGTQFTLTISGVECADAATYYCQQTYSWSGLDNVFGGGTKVEIKRTVAAPSVFIFPPSDEQLKSGTASVVCLLNNFYPREAKVQWKVDNALQSGNSQESVTEQDSKDCTYSLSSTLTLSKADYEKHKVYACEVTHQGLSSPVTKSFNRGEC

SEQ ID NO: 38 - protein, 447 aa, synthetic construct
  REGION 1..447: note = artificially synthesized
QSVEESGGRLVTPGTPLTLTCTVSGFSLSSYAMNWVRQAPGKGLEWIGYIYTSGTTYHANWARGRFTISKTSTTVDLKMTSLTTEDTATYFCARGTGISDYYYWVMDLWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELRRGPKVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 39 - protein, 215 aa, synthetic construct
  REGION 1..215: note = artificially synthesized
AFELTQTPSSVEAAVGGTVTIKCQASQSISTYLAWYQQRPGQPPKLLIYKASTLASGVPSRFKGSGSGTDFTLTISDLECADAATYFCQSYCDGDSVGFGGGTKVEIKRTVAAPSVFIFPPSDEQLKSGTASVVCLLNNFYPREAKVQWKVDNALQSGNSQESVTEQDSKDCTYSLSSTLTLSKADYEKHKVYACEVTHQGLSSPVTKSFNRGEC

SEQ ID NO: 40 - protein, 448 aa, synthetic construct
  REGION 1..448: note = artificially synthesized
QSVEESGGRLVTPGTPLTLTCTVSGFSLSNYAMGWVRQAPGKGLEWIGIISSGGVSYYASWARGRFTISKTSTTVDLKITSPTPEDTGTYFCARGQVPSSDTYYVPFNLWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELRRGPKVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 41 - protein, 217 aa, synthetic construct
  REGION 1..217: note = artificially synthesized
DIAMTQTPASVEAAVGGTVTIKCQASQSIGSDLAWYQQKPGQPPKLLIYRASTLASGVPLRFKGSGSGTQFTLTISDLECADAATYYCQTYYFTISSYGAFGGGTKVEIKRTVAAPSVFIFPPSDEQLKSGTASVVCLLNNFYPREAKVQWKVDNALQSGNSQESVTEQDSKDCTYSLSSTLTLSKADYEKHKVYACEVTHQGLSSPVTKSFNRGEC

SEQ ID NO: 42 - protein, 275 aa, Mus musculus
MKWVTFLLLLFISGSAFSDYKDDDDKLSTSKTIDMELVKRKRIEAIRGQILSKLRLASPPSQGEVPPGPLPEAVLALYNSTRDRVAGESADPEPEPEADYYAKEVTRVLMVDRNNAIYEKTKDISHSIYMFFNTSDIREAVPEPPLLSRAELRLQRLKSSVEQHVELYQKYSNNSWRYLGNRLLTPTDTPEWLSFDVTGVVRQWLNQGDGIQGFRFSAHCSCDSKDNKLHVEINGISPKRRGDLGTIHDMNRPFLLLMATPLERAQHLHSSRHRR

SEQ ID NO: 43 - DNA, 831 bp, Mus musculus
  source 1..831: mol_type = unassigned DNA
atgaagtgggtaacctttctcctcctcctcttcatctccggttctgccttttccgactacaaggatgacgatgacaagctctccacctccaagaccatcgacatggagctggtgaaacggaagcgcatcgaagccatccgtggccagatcctgtccaaactaaggctcgccagtcccccaagccagggggaggtaccgcccggcccgctgcccgaggcggtgctcgctttgtacaacagcacccgcgaccgggtggcaggcgagagcgccgacccagagccggagcccgaagcggactactatgctaaagaggtcacccgcgtgctaatggtggaccgcaacaacgccatctatgagaaaaccaaagacatctcacacagtatatatatgttcttcaatacgtcagacattcgggaagcagtgcccgaacccccattgctgtcccgtgcagagctgcgcttgcagagattaaaatcaagtgtggagcaacatgtggaactctaccagaaatatagcaacaattcctggcgttaccttggtaaccggctgctgacccccactgatacgcctgagtggctgtcttttgacgtcactggagttgtacggcagtggctgaaccaaggagacggaatacagggctttcgattcagcgctcactgctcttgtgacagcaaagataacaaactccacgtggaaatcaacgggatcagccccaaacgtcggggcgacctgggcaccatccatgacatgaaccggcccttcctgctcctcatggccacccccctggaaagggcccagcacctgcacagctcacggcaccggagatgatga

SEQ ID NO: 44 - protein, 324 aa, synthetic construct
  REGION 1..324: note = an artificially synthesized sequence
AKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVKEVSKVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 45 - protein, 107 aa, Mus musculus
RADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 46 - protein, 441 aa, synthetic construct
  REGION 1..441: note = an artificially synthesized sequence
QSLEESGGDLVKPGASLTLTCTASGFSFSSYWMSWVRQAPGKGLEYIGMISIIGTTYYASWAKGRFTISKTSSTTVTLQVTSLTAADTATYFCARGYVSNSNYWGLWGPGTLVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVKEVSKVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 47 - protein, 217 aa, synthetic construct
  REGION 1..217: note = an artificially synthesized sequence
AYDMTQTPASVEVAVGGTVTIKCQASQNIGSNLAWYQQKPGQPPKLLIYYASDLASGVPSRFKGSGSGTRYTLTISGVQCDDAATYYCQQYWSSSNVDNTFGGGTEVVVKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 48 - protein, 441 aa, synthetic construct
  REGION 1..441: note = an artificially synthesized sequence
QSLEESGGDLVKPGASLTLTCTASGFSFSRSYYMCWVRQAPGKGLEWIACIYGGSSGSTWYASWAKGRFTISKTSSTTVTLQMTSLTAADTATYFCARDIGNYYRWWGPGTLVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVKEVSKVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 49 - protein, 219 aa, synthetic construct
  REGION 1..219: note = an artificially synthesized sequence
AVVLTQTTSPVSAPVGGTVTIKCQASKNIYSLLAWYQQKPGQPPKLLIYDASTLASGVPSRFKGSGSGTQFTLTISDLECADAATYYCQSYYDSSSSADLSTFGGGTEVVVKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 50 - protein, 437 aa, synthetic construct
  REGION 1..437: note = an artificially synthesized sequence
QSLEESGGRLVTPGGSLTLTCTVSGIDLSSYAMGWVRQAPGKGLEYIGIISSSGLTDYASWVNGRFTISKTSTTVDLKITSPTTEDTATYFCARIYDLWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELRRGPKVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 51 - protein, 219 aa, synthetic construct
  REGION 1..219: note = an artificially synthesized sequence
DIVMTQTPASVEAAVGGTLTIKCQASQSISDQLSWYQQKPGQPPKLLIYRASTLESGVPSRFKGSGSGTQFTLTISDLECADAATYYCQSYYYSYSSTYGNTFGGGTKVEIKRTVAAPSVFIFPPSDEQLKSGTASVVCLLNNFYPREAKVQWKVDNALQSGNSQESVTEQDSKDCTYSLSSTLTLSKADYEKHKVYACEVTHQGLSSPVTKSFNRGEC

SEQ ID NO: 52 - protein, 437 aa, synthetic construct
  REGION 1..437: note = an artificially synthesized sequence
QSLEESGGRLVTPGGSLTLTCTVSGIDLSSYAMGWVRQAPGKGLEYIGIISNSGLTDYASWVNGRFTISKTSTTVDLKITSLTTEDTATYFCARIYDLWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELRRGPKVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 53 - protein, 219 aa, synthetic construct
  REGION 1..219: note = an artificially synthesized sequence
DIVMTQTPASVEAAVGGTVTIKCQASQSISNQLSWYQQKPGQPPKLLIYRASTLASGVSSRFKGSGSGTQFTLTISDLECAAAATYYCQSYYYSVSSTYGNTFGGGTKVEIKRTVAAPSVFIFPPSDEQLKSGTASVVCLLNNFYPREAKVQWKVDNALQSGNSQESVTEQDSKDCTYSLSSTLTLSKADYEKHKVYACEVTHQGLSSPVTKSFNRGEC

SEQ ID NO: 54 - protein, 117 aa, Oryctolagus cuniculus
QSLEESGGDLVKPGASLTLTCTASGFSFSSYWMSWVRQAPGKGLEYIGMISIIGTTYYASWAKGRFTISKTSSTTVTLQVTSLTAADTATYFCARGYVSNSNYWGLWGPGTLVTVSS

SEQ ID NO: 55 - protein, 110 aa, Oryctolagus cuniculus
AYDMTQTPASVEVAVGGTVTIKCQASQNIGSNLAWYQQKPGQPPKLLIYYASDLASGVPSRFKGSGSGTRYTLTISGVQCDDAATYYCQQYWSSSNVDNTFGGGTEVVVK

SEQ ID NO: 56 - protein, 117 aa, Oryctolagus cuniculus
QSLEESGGDLVKPGASLTLTCTASGFSFSRSYYMCWVRQAPGKGLEWIACIYGGSSGSTWYASWAKGRFTISKTSSTTVTLQMTSLTAADTATYFCARDIGNYYRWWGPGTLVTVSS

SEQ ID NO: 57 - protein, 112 aa, Oryctolagus cuniculus
AVVLTQTTSPVSAPVGGTVTIKCQASKNIYSLLAWYQQKPGQPPKLLIYDASTLASGVPSRFKGSGSGTQFTLTISDLECADAATYYCQSYYDSSSSADLSTFGGGTEVVVK

SEQ ID NO: 58 - protein, 5 aa, Oryctolagus cuniculus
SYWMS

SEQ ID NO: 59 - protein, 16 aa, Oryctolagus cuniculus
MISIIGTTYYASWAKG

SEQ ID NO: 60 - protein, 11 aa, Oryctolagus cuniculus
GYVSNSNYWGL

SEQ ID NO: 61 - protein, 11 aa, Oryctolagus cuniculus
QASQNIGSNLA

SEQ ID NO: 62 - protein, 7 aa, Oryctolagus cuniculus
YASDLAS

SEQ ID NO: 63 - protein, 12 aa, Oryctolagus cuniculus
QQYWSSSNVDNT

SEQ ID NO: 64 - protein, 6 aa, Oryctolagus cuniculus
RSYYMC

SEQ ID NO: 65 - protein, 18 aa, Oryctolagus cuniculus
CIYGGSSGSTWYASWAKG

SEQ ID NO: 66 - protein, 8 aa, Oryctolagus cuniculus
DIGNYYRW

SEQ ID NO: 67 - protein, 11 aa, Oryctolagus cuniculus
QASKNIYSLLA

SEQ ID NO: 68 - protein, 7 aa, Oryctolagus cuniculus
DASTLAS

SEQ ID NO: 69 - protein, 14 aa, Oryctolagus cuniculus
QSYYDSSSSADLST

SEQ ID NO: 70 - protein, 446 aa, synthetic construct
  REGION 1..446: note = an artificially synthesized sequence
QSVEESGGRLVTPGTPLTLTCTASGFSLSNYDMSWVRQAPGKGLEWIGNIHTADTTSYARWAKGRFTISKTSTTVDLKITSPTSEDTATYFCARGGSDFFYYEPYNIWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELRRGPKVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 71 - protein, 217 aa, synthetic construct
  REGION 1..217: note = an artificially synthesized sequence
AYDMTQTPASVEAAVGGTVTVRCQASESIGSRLAWYQQKVGQPPKLLIYDASTLASGVSSRFKGSGSGTEFTLTISDLECDDAATYYCQQYYTSNNVENTFGGGTKVEIKRTVAAPSVFIFPPSDEQLKSGTASVVCLLNNFYPREAKVQWKVDNALQSGNSQESVTEQDSKDCTYSLSSTLTLSKADYEKHKVYACEVTHQGLSSPVTKSFNRGEC

SEQ ID NO: 72 - protein, 447 aa, synthetic construct
  REGION 1..447: note = an artificially synthesized sequence
QEQLKESGGGLVTPGGTLTLTCTVSGFSLSNYDMSWVRQAPGKGLEWIGAIYTAGTTSYASWAKGRFTISKTSTTVDLKITSPTTEDTATYFCARGGSSFFYYAAHNIWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELRRGPKVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 73 - protein, 217 aa, synthetic construct
  REGION 1..217: note = an artificially synthesized sequence
AYDMTQTPASVEAAVGGTVTIKCQASQSIGSNLAWYQQKPGQPPKLLIYDASKLPSGVPSRFKGSGSGTEFTLTISDLECDDAATYYCQQYYTTSDVDNTFGGGTKVEIKRTVAAPSVFIFPPSDEQLKSGTASVVCLLNNFYPREAKVQWKVDNALQSGNSQESVTEQDSKDCTYSLSSTLTLSKADYEKHKVYACEVTHQGLSSPVTKSFNRGEC

SEQ ID NO: 74 - protein, 446 aa, synthetic construct
  REGION 1..446: note = an artificially synthesized sequence
QSVEESGGRLVTPGTPLTLTCTVSGFSLSNYAMTWVRQAPGKGLEWIGVIYAGGNTYYANWVNGRFTISKTSTTVDLKITSPTTEDTATYFCARDVGSSSSGVYFNIWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELRRGPKVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 75 - protein, 217 aa, synthetic construct
  REGION 1..217: note = an artificially synthesized sequence
AYDMTQTPASVEVAVGGTVTIKCQASQSIGSRLAWYQQKPGQPPKLLIYDASTLASGVSSRFKGSGSGTEFTLTISGVECADAATYYCQQDYSYRHVDNIFGGGTKVEIKRTVAAPSVFIFPPSDEQLKSGTASVVCLLNNFYPREAKVQWKVDNALQSGNSQESVTEQDSKDCTYSLSSTLTLSKADYEKHKVYACEVTHQGLSSPVTKSFNRGEC

SEQ ID NO: 76 - protein, 118 aa, Oryctolagus cuniculus
QSVEESGGRLVTPGTPLTLTCTASGFSLSNYDMSWVRQAPGKGLEWIGNIHTADTTSYARWAKGRFTISKTSTTVDLKITSPTSEDTATYFCARGGSDFFYYEPYNIWGPGTLVTVSS

SEQ ID NO: 77 - protein, 110 aa, Oryctolagus cuniculus
AYDMTQTPASVEAAVGGTVTVRCQASESIGSRLAWYQQKVGQPPKLLIYDASTLASGVSSRFKGSGSGTEFTLTISDLECDDAATYYCQQYYTSNNVENTFGGGTKVEIK

SEQ ID NO: 78 - protein, 119 aa, Oryctolagus cuniculus
QEQLKESGGGLVTPGGTLTLTCTVSGFSLSNYDMSWVRQAPGKGLEWIGAIYTAGTTSYASWAKGRFTISKTSTTVDLKITSPTTEDTATYFCARGGSSFFYYAAHNIWGPGTLVTVSS

SEQ ID NO: 79 - protein, 110 aa, Oryctolagus cuniculus
AYDMTQTPASVEAAVGGTVTIKCQASQSIGSNLAWYQQKPGQPPKLLIYDASKLPSGVPSRFKGSGSGTEFTLTISDLECDDAATYYCQQYYTTSDVDNTFGGGTKVEIK

SEQ ID NO: 80 - protein, 118 aa, Oryctolagus cuniculus
QSVEESGGRLVTPGTPLTLTCTVSGFSLSNYAMTWVRQAPGKGLEWIGVIYAGGNTYYANWVNGRFTISKTSTTVDLKITSPTTEDTATYFCARDVGSSSSGVYFNIWGPGTLVTVSS

SEQ ID NO: 81 - protein, 110 aa, Oryctolagus cuniculus
AYDMTQTPASVEVAVGGTVTIKCQASQSIGSRLAWYQQKPGQPPKLLIYDASTLASGVSSRFKGSGSGTEFTLTISGVECADAATYYCQQDYSYRHVDNIFGGGTKVEIK

SEQ ID NO: 82 - protein, 5 aa, Oryctolagus cuniculus
NYDMS

SEQ ID NO: 83 - protein, 16 aa, Oryctolagus cuniculus
NIHTADTTSYARWAKG

SEQ ID NO: 84 - protein, 13 aa, Oryctolagus cuniculus
GGSDFFYYEPYNI

SEQ ID NO: 85 - protein, 11 aa, Oryctolagus cuniculus
QASESIGSRLA

SEQ ID NO: 86 - protein, 7 aa, Oryctolagus cuniculus
DASTLAS

SEQ ID NO: 87 - protein, 12 aa, Oryctolagus cuniculus
QQYYTSNNVENT

SEQ ID NO: 88 - protein, 5 aa, Oryctolagus cuniculus
NYDMS

SEQ ID NO: 89 - protein, 16 aa, Oryctolagus cuniculus
AIYTAGTTSYASWAKG

SEQ ID NO: 90 - protein, 13 aa, Oryctolagus cuniculus
GGSSFFYYAAHNI

SEQ ID NO: 91 - protein, 11 aa, Oryctolagus cuniculus
QASQSIGSNLA

SEQ ID NO: 92 - protein, 7 aa, Oryctolagus cuniculus
DASKLPS

SEQ ID NO: 93 - protein, 12 aa, Oryctolagus cuniculus
QQYYTTSDVDNT

SEQ ID NO: 94 - protein, 5 aa, Oryctolagus cuniculus
NYAMT

SEQ ID NO: 95 - protein, 16 aa, Oryctolagus cuniculus
VIYAGGNTYYANWVNG

SEQ ID NO: 96 - protein, 13 aa, Oryctolagus cuniculus
DVGSSSSGVYFNI

SEQ ID NO: 97 - protein, 11 aa, Oryctolagus cuniculus
QASQSIGSRLA

SEQ ID NO: 98 - protein, 7 aa, Oryctolagus cuniculus
DASTLAS

SEQ ID NO: 99 - protein, 12 aa, Oryctolagus cuniculus
QQDYSYRHVDNI